(19) United States
(12) Patent Application Publication
(10) Pub. No.: US 2026/0256912 A1
(43) Pub. Date: Sep. 3, 2026

(54) CD74 CAR-T THERAPY AND METHODS OF USE THEREOF

(71) Applicant: OHIO STATE INNOVATION FOUNDATION, Columbus, OH (US)

(72) Inventors: Lapo ALINARI, Powell, OH (US); Wing Keung CHAN, Dublin, OH (US)

(21) Appl. No.: 19/489,456

(22) PCT Filed: Jun. 14, 2024
(86) PCT No.: PCT/US2024/033945

§ 371 Date: Dec. 2, 2025

Related U.S. Application Data
(60) Provisional application No. 63/521,467, filed on Jun. 16, 2023.

(51) Int. Cl. A61K 40/31; A61K 35/17; A61K 40/11; A61K 40/42; A61K 45/06; A61P 35/00; C07K 14/705; C07K 16/28; C12N 5/0783

(52) U.S. Cl. CPC A61K 40/31; A61K 35/17; A61K 40/11; A61K 40/4213; A61K 45/06; A61P 35/00; C07K 14/7051; C07K 14/70596; C07K 16/2833; C12N 5/0636; A61K 2239/13; A61K 2239/17; A61K 2239/22; C07K 2317/622; C12N 2510/00

(57) ABSTRACT

The present disclosure provides for a chimeric antigen receptor (CAR) polypeptide comprising a CD74 antigen binding domain, a transmembrane domain, an intracellular signaling domain, and a co-stimulatory signaling region. Further provided herein is an isolated nucleic acid encoding the recombinant polypeptide as disclosed herein, a vector comprising the isolated nucleic acid, and a cell comprising the vector. Also provided herein is a method of treating lymphoma in a subject in need thereof, the method comprising administering to the subject a therapeutically effective amount of the chimeric antigen receptor polypeptide as disclosed herein. Further described herein is a method of reducing tumor activity in a subject with lymphoma, the method comprising administering to the subject a therapeutically effective amount of the chimeric antigen receptor polypeptide as described herein.

### CROSS-REFERENCE TO RELATED APPLICATIONS

This application claims the benefit of priority to, and the benefit of, U.S. Provisional Application No. 63/521,467 filed on Jun. 16, 2023, the disclosure of which is hereby expressly incorporated by reference herein in its entirety.

### REFERENCE TO SEQUENCE LISTING

This sequence listing submitted on Jun. 14, 2024, as an .XML file entitled “103361-486WO1_ST26.xml” created on Jun. 13, 2024, and having a file size of 153,628 bytes is hereby incorporated by reference pursuant to 37 C.F.R. § 1.52(e)(5).

### FIELD

The present disclosure relates to chimeric antigen receptors and uses thereof.

### BACKGROUND

Mantle cell lymphoma (MCL) is an incurable subtype of B-cell non-Hodgkin's lymphoma (NHL) characterized by an increasing incidence over the past two decades. MCL is conventionally classified into 3 morphological variants: classic, blastoid, and pleomorphic, with the last 2 being considered more aggressive and associated with poorer prognosis. Immunophenotypically, MCL cells express pan-B cell markers such as CD19 and CD20 and are usually CD5+, CD10−, CD23− and FMC7+ though variations in subsets of patients are reported. Molecularly, MCL is characterized by the translocation (11; 14) which results in Cyclin D1 overexpression and cell cycle dysregulation. In addition, secondary chromosomal alterations targeting genes involved in the regulation of cell cycle, DNA damage response, and cell survival pathways are frequently described in this disease. These include deletions of TP53, BCL2L11, RB1, and ATM, amplification of BCL2 and c-MYC.

Younger MCL patients are treated with intensive chemo-immunotherapy regimens followed by autologous stem cell transplant consolidation. However, the majority of older patients are treated with less intensive approaches. Outside allogeneic stem cell transplant, treatment of patients with relapsed or refractory MCL is typically palliative and primarily include covalent or non-covalent Bruton Tyrosine Kinase (BTK) inhibitors, and Bcl2 antagonists. Though the disease responds to initial treatment, relapse inevitably occurs, and prognosis is overall poor. In 2020, the first and only anti-CD19 CAR-T therapy (Tecartus KTE-X19) was approved for the treatment of relapsed and refractory MCL. A Phase II clinical trial conducted across multiple centers enrolled 74 patients with a history of relapsed or refractory MCL, who had previously been treated with ibrutinib (covalent BTK inhibitor) and up to five other lines of treatments. The patients were given a single dose of 2×10⁶/kg KTE-X19 CAR-T cells. After a median follow-up of 35.6 months, the overall response rate remained high at 91% with 68% of the 68 treated patients achieving a complete remission. However, the progression free survival and the overall survival were only 25.8 and 46.6 months, respectively, suggesting that a significant portion of these patients needed additional treatment.

CD74 is a nonpolymorphic type II transmembrane glycoprotein with its N-terminus domain facing the cytosol that functions as an MHC class II chaperone promoting exogenous antigen processing and presentation. In the endoplasmic reticulum (ER), a CD74 trimer binds to three MHCII alpha-beta dimers forming a nonameric structure which then egresses the ER and translocates to the cell surface. CD74 also functions as the receptor for the macrophages migration inhibitory factor (MIF), a proinflammatory cytokine, and has been previously shown to promote normal B cell proliferation and survival by activating downstream pathways such as NF-kB and B-cell receptor/PI3K/Akt signaling. CD74 is produced in molar excess in comparison to MHCII which results in its abundant free-loaded expression on the cell surface and is degraded through a proteolytic mechanism initiated by the cytoplasmic cleavage of its N-terminus domain by Signal Peptide Peptidase-Like 2A (SPPL2a). Consistent with its role on antigen presentation, CD74 is expressed on professional antigen presenting cells such as macrophages, dendritic cells, and B cells as well as epithelial cells under inflammatory conditions. CD74 is expressed at much higher levels on a variety of hematologic malignancies such as B and T cell lymphomas, multiple myeloma (MM), and chronic lymphocytic leukemia (CLL) in addition to solid tumors of the gastrointestinal tract. In a phase I clinical trial conducted in 22 patients with relapsed/refractory B-cell NHL, milatuzumab was overall well tolerated with the most common toxicities being infusion reaction and cytopenias but demonstrated very limited clinical activity with no objective response reported. In a phase I-II clinical trial conducted in 8 patients with relapsed/refractory CLL, milatuzumab was overall well tolerated with improvement in performance status observed in most patients and a modest response in hematological parameters but with no patients meeting the iwCLL criteria for partial or complete response.

The composition and methods disclosed herein address these and other needs.

### SUMMARY

In accordance with the purposes of the disclosed materials and methods, as embodied and broadly described herein, the disclosed subject matter, in one aspect, relates to chimeric antigen receptor polypeptides and methods related thereto.

Thus, in one example, a chimeric antigen receptor (CAR) polypeptide is provided, including a CD74 antigen binding domain, a transmembrane domain, an intracellular signaling domain, and a co-stimulatory signaling region.

In a further example, an isolated nucleic acid encoding the recombinant polypeptide is provided.

Additionally, a method of treating lymphoma in a subject in need thereof is provided, including administering to the subject a therapeutically effective amount of the chimeric antigen receptor polypeptide as disclosed herein.

Further, a method of reducing tumor activity in a subject with lymphoma is provided, including administering to the subject a therapeutically effective amount of the chimeric antigen receptor polypeptide as described herein.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying figures, which are incorporated in and constitute a part of this specification, illustrate several aspects described below.

FIGS. 1A-1G show that scFV sequence optimization yields an anti-CD74 CAR (74bbz)-T cell with superior functions. FIG. 1A shows the design of CD74 CAR lentiviral vector. CD74 scFV: anti-CD74 scFV ended with CD8a hinge region; TM: CD8a transmembrane domain; 4-1BB: signaling domain of 4-1BB molecule; CD3ζ: CD3 zeta chain; T2A: self-cleaving peptide from those asigna virus 2A; reporter: GFP or tEGFR reporter gene. FIG. 1B shows the distribution of mutations on the V_{H}/V_{L} chain (right) and framework (FR)/complementarity-determining region (CDR) of the scFV on the 74bbz CAR. FIG. 1C shows the binding affinity of 74bbz CAR mutants displayed on Jurkat cell to CD74 ECD-Fc. The Jurkat CAR mutant cells were reacted to the CD74-ECD-Fc and anti-Fc-APC by flow cytometry. The red bar is the parent 74bbz. All the numbers on the x-axis are the CAR clone numbers. FIG. 1D shows the early T cell activation marker CD69 expression on Jurkat CAR mutant cells. The Jurkat CAR mutant cells were incubated with Mino cells at an effector-to-target (ET) ratio of 1:2 for 6 hours before the Jurkat CAR mutant cells were stained for CD69. FIG. 1E shows the repeated antigen stimulation assay for Jurkat CAR mutant cells. The Jurkat CAR mutant cells were weekly stimulated with irradiated Mino cells for three weeks and cultured under suboptimal culture conditions. The cell proliferation on Jurkat CAR mutant cells were assayed and the percentage of increased cell numbers were normalized as fold increase compared to Jurkat cells with parent 74bbz cells. FIG. 1F shows specific lysis of the Mino cells by Jurkat CAR mutant cells. Jurkat CAR mutant cells were cocultured with Mino cells at ET ratio of 5:1 for 24 hours before the specific lysis were analyzed. FIG. 1G shows a four-way Venn diagram identified 2 clones of 74bbz CAR with the superiority over four aspects of CAR-T cell: CD69 activation (yellow), cytotoxicity (green), proliferation (red), and binding to CD74 (blue).

FIGS. 2A-2B shows 42105-74bbz CAR-T cells induced the highest specific lysis among mutants against Mino and JeKo-1 cells. FIG. 2A shows three 74bbz CAR mutants were expressed on primary CD4/8 T cells (n=3) and used as effector cells in the co-culture of Mino (left) and JeKo-1 (right). Mutants 543, 5311 and 42105 were compared to the parent 74bbz CAR-T cells. FIG. 2B shows 42105-74bbz CAR-T cells had the highest specific lysis signal-to-noise as determined by the specific lysis on CD74+ Mino cells over the CD74-SUDHL-1 cells. The ratios from all three mutants were compared to that of the parent. Data are mean±S.E.M. from three independent experiments. *p<0.05; **p<0.01; ***p<0.001.

FIGS. 3A-3C: Cytotoxicity and IFN-γ production of optimized 42105-74bbz CAR-T cells against MCL cells. FIG. 3A shows the expression of CD74 on MCL cell lines. Numbers on the top right corner: surface antigen density. The data presented are one representative of three independent experiments. FIG. 3B shows the cytotoxicity assay of optimized 42105-74bbz CAR-T cells against 6 different MCL cell lines. Untransduced T cells (UTT, red circle) or optimized 42105-74bbz CAR-T cells (blue, triangles) were co-cocultured with target cells at ET ratio od 5 for 24 hours before the specific lysis was determined. CD74− T cell lymphoma cell line SUDHL-1 was used as a negative control. Each dot represents one experimental result from each healthy donor CAR-T cells. FIG. 3C shows IFN-γ production from 42105-74bbz CAR-T cells after co-cultured with indicated cell for 24 hours. Effector alone was used as the negative control. Results were presented as mean±SEM from three independent experiments. *p<0.05; **p<0.01; ***p<0.001.

FIGS. 4A-4C show the activity of optimized 42105-74bbz CAR-T cells against primary MCL patient lymphoma cells. FIG. 4A shows the expression of CD74 on MCL patient's lymphoma cells identified by CD5+CD19+ MCL subsets. Numbers on the top corners are the antigen density. The data presented are one representative of three independent experiments. FIG. 4B shows the susceptibility of three MCL patient lymphoma cells to the 42105-74bbz CAR-T cells. MCL patient cells were used as target cells in the co-culture of optimized 74bbz CAR-T cells at ET ratio of 5:1 for 24 hours. Results were presented as mean±SEM from three independent experiments. **p<0.01; ***p<0.001. FIG. 4C shows antigenic dependence of CD74 on the specific lysis of 42105-74bbz CAR-T cells. The CD74 surface antigen density MESF and specific lysis values of JeKo-1, Mino, UPN, Granta-519, Z138, SUDHL-1 (left), and Patient 1-5 (right) were used to perform the correlation calculations for the correlation coefficient R² and the p-value.

FIGS. 5A-5G shows that CD74 expression remains on CD33+ myeloid subset of normal PBMCs. FIG. 5A shows CD74 antigen density on MCL cell (Table 1) was significantly higher than PBMCs from healthy blood donors, while no statistical significance between PBMCs and negative control SUDHL-1 was found. FIG. 5B shows histogram plots of CD74 expression on CD33+ and CD33− cells (shaded gray) compared to the isotype staining control (dotted line). CD74 density on CD33+ and CD33− cells were summarized from 3 independent donors (right). FIG. 5C shows specific lysis of normal CD33+ cells when used as target cells for 42105-74bbz CAR-T cells. Mino cells were used as a positive control target. FIG. 5D shows CD74 expression on CD14+ monocyte (shaded gray) compared to the isotype staining control (dotted line). A summary of CD74 density on monocytes from 3 independent donors was plotted (right). FIG. 5E shows susceptibility of monocyte to 42105-74bbz CAR-T cells (white) and UTT (black). FIG. 5F shows a subset of T_{H} cell (CD4+ T cells), T_{CYTO} cell (CD8+ T cells), B cell and NK cell expressed CD74. Data are from one representative of three independent experiments. CD74 densities on B cell, T_{H} cell, T_{CYTO} cell and NK cell were all below 10,000 molecules per cell. Numbers are the median molecules per cell. Data are from three independent donors (bottom). FIG. 5G shows the susceptibility of resting and activated T/B cells to 42105-74bbz CAR-T cells (white) and UTT cells (black). Results are mean S.E.M. from three independent experiments. *p<0.05; **p<0.01; ***p<0.001.

FIGS. 6A-6B show 42105-74bbz CAR-T cells did not deplete normal immune cells. FIG. 6A shows autologous 42105-74bbz CAR-T cell number peaked on Day 18 post CAR-T cell injection in the humanized NSG mice. FIG. 6B shows that there were no significant changes on the absolute cell numbers of B cell, monocyte, granulocytic myeloid suppressor cells (G-MDSC), monocytic myeloid suppressor cells (M-MDSC) and NK cells. All human cells were identified by human CD45+. B: CD19+; Monocyte: LIN-CD14+; G-MDSC: HLA-DR+CD14-CD33+; M-MDSC: HLA-DR+CD14+CD33+; NK: CD3-CD56+. Mice received either UTT cells (n=5) and 42105-74bbz (n=7). Bars show the median cell number.

FIGS. 7A-7B show 42105-74bbz CAR-T cells provided a longer survival than the parent in a CD19 CAR-T relapsed patient-derived xenograft MCL NSG model. FIG. 7A shows Kaplan-Meier curves of mice untreated (blue) and treated with parent 74bbz CAR-T cells (green) and 42105-74bbz CAR-T cells (red). 8 mice were used in each group. The lymphoma specific survival was determined by the presence of tumor cells in any organs in situ. *p<0.05; **p<0.01; ***p<0.001. FIG. 7B shows that 42105-74bbz CAR-T cells were more abundant in spleen at ERC than the parent 74bbz CAR-T cell treated mice. Lines are medians of the log absolute number of cells in 100 μL blood or 1×10⁶ splenocytes.

FIGS. 8A-8B show that 42105-74bbz CAR-T cells are not inferior to the 19bbz CAR-T cells in prolonging the survival in a preclinical MCL NSG mouse model. FIG. 8A shows mice were engrafted with Mino cells subcutaneously and randomized into groups of 10 for tumor alone (blue), tumor+UTT cells (green), tumor+19bbz CAR-T cells (black) and tumor+42105-74bbz CAR-T cells (red). Table (bottom) was a summary of p-values in survival comparisons among all groups. FIG. 8B shows that mice received 42105-74bbz CAR-T cells had the least median log absolute numbers of tumor cells in blood and spleen harvested when mice reached ERC. While the median log absolute number of circulating CAR-T cells in 74bbz CAR-T cells treated mice was higher than that of 19bbz CAR-T cells treated mice, similar numbers of CAR-Ts in both 19bbz and 74bbz CAR-T cells treated mice were found.

FIGS. 9A-9B show in silico modeling of CD74-anti-CD74 scFV interaction. (A) FIG. 9A shows the best generated models of CD74-anti-CD74 scFV interaction shown by the lowest HADDOCK score as a function of RMSD. The blue cluster was picked for further in silico mutagenesis. (B) FIG. 9B shows the visualization of CD74-anti-CD74 scFV interaction. Red: CD74 trimer; Blue: anti-CD74 scFV.

FIGS. 10A-10C shows the creation of the 74bbz mutant clones. FIG. 10A shows the GFP+ cells of the 74bbz mutant and parent CAR expressing Jurkat cells were sorted at the same intensity by flow cytometry. FIG. 10B shows an immunoblot of CD3ζ to show the expressing of parent, 543, 5311, 42105-74bbz clones. Endogenous CD3ζ was detected at 15 kDa while the chimeric CD3ζ on CAR was detected at 55 kDa. (C) FIG. 10C shows CD74-ECD-Fc fusion protein stained by Coomassie blue staining.

FIG. 11 shows the expression of CD74 after activation on T cell and B cell. T cells and B cells isolated from PBMCs of 3 healthy blood donors were either untreated or activated by CD3/CD28 soluble antibody and IL-2 for T cells, and LPS (10 ng/mL)/anti-IgM (10 ug/mL) for B cells. The numbers on the top right corners were MFI of the CD74 expression on resting (red) and activated (blue) cells. One representative of 3 healthy blood donors was shown.

FIG. 12 shows the absolute cell numbers of B cell, monocyte, granulocytic myeloid suppressor cells, monocytic myeloid suppressor cells and NK cells in humanized NSG mice on Day 3, 11 and 23 post UTT or 74bbz CAR-T engraftment. All human cells were identified by human CD45+. B: CD19+; Monocyte: LIN-CD14+; G-MDSC: HLA-DR+CD14-CD33+; M-MDSC: HLA-DR+CD14+CD33+; NK: CD3-CD56+. Mice received either UTT cells (n=5) and 42105-74bbz (n=7). Bars show the median cell number.

FIGS. 13A-13B show cHL malignant cells (HRS) expressing abundant levels of CD74. FIG. 13A shows surface expression of CD74 relative to isotype on cHL cell lines and HRS cells from a primary cHL sample using flow cytometry. FIG. 13B shows immunohistochemical (IHC) staining of lymph nodes involved by cHL (at 200× and 600×) showing strong surface and cytoplasmic CD74 expression (brown) in HRS cells (black arrows) as well as mild to moderate positivity in background normal immune cell subsets.

FIG. 14 shows 74bbz CAR-Ts effectively kill cHL cells in vivo. FIG. 14 shows NSG mice were engrafted with 1×10⁶ KMH2 cells via tail vein injection. Once bioluminescence signal reached detectable range by IVIS (2 weeks after engraftment), mice were randomized into groups of 5 for tumor alone (blue), UTT cells (5×10⁶, red), 74bbz CAR-T cells (5×10⁶, green). Mice treated with 74bbz CAR-T cells did not have any evidence of disease by IVIS and flow cytometry in the peripheral blood at day 70 when the experiment was terminated due to initial signs of GVHD. 50% of mice in the UTT group were alive at day 70 (p=0.03).

FIGS. 15A-15B show 74bbz CAR-Ts efficiently kill TAMs2. FIG. 15A shows surface expression of CD74 relative to the isotype on peripheral blood monocyte-derived TAMs stimulated with the M2-polarizing cytokine M-CSF. FIG. 15B shows TAMs2 were co-cultured with 74bbz CAR-T cells (or UTT control) at ET ratio of 5:1 for 24 hours. Cell death was measured by the AK release cytotoxicity assay. Results were presented as mean±SEM from three independent experiments. **p<0.01; ****p<0.0001.

FIGS. 16A-16B show cell lines and primary malignant cells derived from T cell lymphoma patients aberrantly express CD74. FIG. 16A shows T cell lymphoma cell lines were stained with isotype control (green) or anti-CD74 (red) antibody. FIG. 16B shows primary T cell lymphoma patient cells with aberrant CD2+, CD7− (gated in red) are CD74+.

### DETAILED DESCRIPTION

In accordance with the purposes of the disclosed materials and methods, as embodied and broadly described herein, the disclosed subject matter relates to CD74 chimeric antigen receptor polypeptides and methods related thereto.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

The term “subject” preferably refers to a human. However, the term “subject” can also refer to non-human animals, preferably mammals such as dogs, cats, horses, cows, pigs, sheep and non-human primates, among others.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control (e.g., an untreated tumor).

The term “treating” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “peptide,” “protein,” and “polypeptide” are used interchangeably to refer to a natural or synthetic molecule comprising two or more amino acids linked by the carboxyl group of one amino acid to the alpha amino group of another.

The term “nucleic acid” refers to a natural or synthetic molecule comprising a single nucleotide, or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to natural or synthetic antibodies that selectively bind a target antigen. The term includes polyclonal and monoclonal antibodies. In addition to intact immunoglobulin molecules, also included in the term “antibodies” are fragments or polymers of those immunoglobulin molecules, and human or humanized versions of immunoglobulin molecules that selectively bind the target antigen.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus and/or N-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

The term “domain” refers to a specific physical region or amino acid sequence in a protein which is associated with a particular function or corresponding segment of DNA.

A “spacer”, also referred to herein as a “linker”, as used herein refers to a peptide that joins the proteins comprising a fusion protein. Generally, a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

### Compositions

### Chimeric Antigen Receptor Polypeptides

Provided herein is a chimeric antigen receptor (CAR) polypeptide comprising a CD74 antigen binding domain, a transmembrane domain, an intracellular signaling domain, and a co-stimulatory signaling region.

Chimeric antigen receptors (CARs) are receptor proteins that have been engineered to give T cells the ability to target a specific antigen. The receptors are chimeric in that they combine both antigen binding and T cell activating functions into a single receptor.

CD74 is a protein that in humans is encoded by the CD74 gene. The CD74 gene is a Protein Coding gene and is associated with diseases that include, but are not limited to, lymphoma and solid tumor of the gastrointestinal tract. Related pathways include but are not limited to innate immune system and immune response antigen presentation by major histocompatibility complex (MHC) class II.

Antigen binding domain refers to the region of an antibody that binds to antigens. It can comprise one constant and one variable domain of each of the heavy chain and light chain.

A transmembrane domain is a membrane-spanning protein domain.

A hinge domain is a flexible amino acid stretch of that is present in some immunoglobulins. The hinge domain provides segmental flexibility and can allow for crosslinking of two antigens or binding of two antigenic determinants on the same antigen molecule.

The intracellular signaling domain communicates via protein-protein interactions against effector proteins, which in turn pass a signal to the destination.

The co-stimulatory signaling region refers to a portion of the CAR comprising the intracellular domain of a costimulatory molecule. A costimulatory molecule is a cell surface molecule, other than an antigen receptor or their ligands, that is required for an efficient response of lymphocytes to an antigen.

In some examples, the CD74 antigen binding domain is a single-chain variable fragment (scFv) of an antibody that specifically binds to CD74.

A single chain variable fragment (scFv) is a fusion protein of the variable regions of the heavy and light chains of immunoglobulins, connected with a short linker peptide.

In further examples, the co-stimulatory region comprises a cytoplasmic domain of costimulatory molecule 4-1BB.

In some examples, the cytoplasmic domain comprises CD28, 4-1BB, CD278, CD134, CD27, CD40, CD40L, TLRs, or any combination thereof. In further examples, the co-stimulatory region comprises 1, 2, 3, or 4 cytoplasmic domains of one or more cytoplasmic molecules.

4-1BB is a co-stimulatory glycoprotein receptor that is part of the tumor necrosis factor superfamily. It is an inducible cell surface receptor that is expressed in the presence of activating stimuli and functions in cell signaling during T cell activation and proliferation.

In certain examples, the intracellular signaling domain comprises a CD3 zeta (CD3ζ) signaling domain.

T-cell surface glycoprotein CD3 zeta (CD3ζ) chain, also known as T-cell receptor T3 zeta chain or CD247 (Cluster of Differentiation 247), is a protein that in humans is encoded by the CD247 gene.

In specific examples, the CAR polypeptide is defined by the formula:

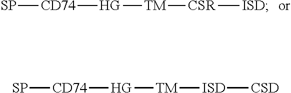

wherein “SP” represents a signal peptide, wherein “CD74” represents a CD74-binding region, wherein “HG” represents an optional hinge domain, wherein “CSR” represents a co-stimulatory signaling region, wherein “ISD” represents an intracellular signaling domain, and wherein “-” represents an optional bivalent linker.

A signal peptide is a short amino acid sequence that controls protein secretion and translocation.

A bivalent linker is a single chemical entity composed of two pharmacophores covalently linked by a spacer of variable size.

In some examples, the scFv of an antibody that specifically binds to CD74 comprises a sequence with at least 60% (e.g., at least 65%, at least 70%, at least 75%, at least 80%, at least 85%, at least 90%, at least 95%, 100%) identity to SEQ ID NOS: 2-7 or a fragment thereof. In further examples, the scFv of an antibody that specifically binds to CD74 comprises a sequence with at least 60% (e.g., at least 65%, at least 70%, at least 75%, at least 80%, at least 85%, at least 90%, at least 95%, 100%) identity to SEQ ID NO: 2 or a fragment thereof.

In certain examples, the scFv of an antibody that specifically binds to CD74 comprises a sequence with at least 60% (e.g., at least 65%, at least 70%, at least 75%, at least 80%, at least 85%, at least 90%, at least 95%, 100%) identity to SEQ ID NOS: 8-12 or a fragment thereof. In specific examples, the scFv of an antibody that specifically binds to CD74 comprises a sequence with at least 60% (e.g., at least 65%, at least 70%, at least 75%, at least 80%, at least 85%, at least 90%, at least 95%, 100%) identity to SEQ ID NO: 8 or a fragment thereof.

In some examples, the scFv of an antibody that specifically binds to CD74 comprises a sequence with at least 60% (e.g., at least 65%, at least 70%, at least 75%, at least 80%, at least 85%, at least 90%, at least 95%, 100%) identity to SEQ ID NOS: 13-14 or a fragment thereof. In further examples, the scFv of an antibody that specifically binds to CD74 comprises a sequence with at least 60% (e.g., at least 65%, at least 70%, at least 75%, at least 80%, at least 85%, at least 90%, at least 95%, 100%) identity to SEQ ID NO: 13 or a fragment thereof.

In certain examples, the scFv of an antibody that specifically binds to CD74 comprises a sequence with at least 60% (e.g., at least 65%, at least 70%, at least 75%, at least 80%, at least 85%, at least 90%, at least 95%, 100%) identity to a SEQ ID NO: of Table 4.

A complementarity-determining region (CDR) is a part of the variable chains in immunoglobulins (antibodies) and T cell receptors. CDRs are the most variable part of the molecules and are therefore important to the diversity of antigen specificities generated by lymphocytes. There are three CDRs (CDR1, CDR2, and CDR3) arranged non-consecutively on the amino acid sequence of a variable domain of an antigen receptor. In some examples, antigen receptors are composed of two variable domains (on two different polypeptide chains: the heavy chain variable region and the light chain variable region) and therefore there are six CDRs for each antigen receptor.

A heavy chain variable region (VH) is the large polypeptide subunit of an antibody.

A light chain variable region (VL) is the small polypeptide subunit of an antibody.

In further examples, the antibody comprises a heavy chain variable region (VH) comprising a sequence with at least 60% (e.g., at least 65%, at least 70%, at least 75%, at least 80%, at least 85%, at least 90%, at least 95%, 100%) identity to SEQ ID NOS: 74, 75, and 76, and a light chain variable region (VL) comprising a sequence with at least 60% identity to SEQ ID NOS: 77, 78, and 79.

In some embodiments, the antibody comprises CDRs from a heavy chain variable region (VH) comprising SEQ ID NO: 74 (CDR1), SEQ ID NO: 75 (CDR2), SEQ ID NO: 76 (CDR3), and CDRs from a light chain variable region (VL) comprising SEQ ID NO: 77 (CDR1), SEQ ID NO: 78 (CDR2), and SEQ ID NO: 79 (CDR3).

In some embodiments, the antibody comprises CDRs from a heavy chain variable region (VH) comprising a sequence with at least 60% (e.g., at least 65%, at least 70%, at least 75%, at least 80%, at least 85%, at least 90%, at least 95%, 100%) identity to SEQ ID NOS: 80, 81, and 82, and CDRs from a light chain variable region (VL) comprising a sequence with at least 60% identity to SEQ ID NOS: 83, 84, and 85.

In some embodiments, the antibody comprises CDRs from a heavy chain variable region (VH) comprising SEQ ID NO: 80 (CDR1), SEQ ID NO: 81 (CDR2), SEQ ID NO: 82 (CDR3), and CDRs from a light chain variable region (VL) comprising SEQ ID NO: 83 (CDR1), SEQ ID NO: 84 (CDR2), and SEQ ID NO: 85 (CDR3).

In some embodiments, the antibody comprises CDRs from a heavy chain variable region (VH) comprising a sequence with at least 60% (e.g., at least 65%, at least 70%, at least 75%, at least 80%, at least 85%, at least 90%, at least 95%, 100%) identity to SEQ ID NOS: 86, 87, 88, and CDRs from a light chain variable region (VL) comprising a sequence with at least 60% (e.g., at least 65%, at least 70%, at least 75%, at least 80%, at least 85%, at least 90%, at least 95%, 100%) identity to SEQ ID NOS: 89, 90, and 91.

In some embodiments, the antibody comprises CDRs from a heavy chain variable region (VH) comprising SEQ ID NO: 86 (CDR1), SEQ ID NO: 87 (CDR2), SEQ ID NO: 88 (CDR3), and CDRs from a light chain variable region (VL) comprising SEQ ID NO: 89 (CDR1), SEQ ID NO: 90 (CDR2), and SEQ ID NO: 91 (CDR3).

In some embodiments, the antibody comprises a heavy chain variable region comprising a sequence with at least 60% (e.g., at least 65%, at least 70%, at least 75%, at least 80%, at least 85%, at least 90%, at least 95%, 100%) identity to a SEQ TD NO. of Table 1 and a light chain variable region comprising a sequence with at least 60% (e.g., at least 65%, at least 70%, at least 75%, at least 80%, at least 85%, at least 90%, at least 95%, 100%) identity to a SEQ ID NO. of Table 1.

TABLE 1
CDR SEQ ID NOS.
CDRs

| Sequence | Variable | SEQ | |
|---|---|---|---|
| Name | Region | ID NO. | Sequence |
| 42105-01 | VH | 74 | CDR1: NYGVS |
| | | 75 | CDR2: VINPNTGEPYYNSALKS |
| | | 76 | CDR3: SRGKNEAWFAY |
| | VL | 77 | CDR1: RASQSLVHRNGNTYLN |
| | | 78 | CDR2: TVSRLHS |
| | | 79 | CDR3: SQSSHVPPT |
| 5311-01 | VH | 80 | CDR1: NYGVN |
| | | 81 | CDR2: WINPNTGEPTYDDDFKG |
| | | 82 | CDR3: SRGKNEAWFAY |
| | VL | 83 | CDR1: RASQSLVHRNGNTYLN |
| | | 84 | CDR2: TVSRLHS |
| | | 85 | CDR3: SQSSHVPPT |
| 543 | VH | 86 | CDR1: SYGMH |
| | | 87 | CDR2: WVAVIWYDGSNKYYADSVKG |
| | | 88 | CDR3: GGTLVRGAMYGTDV |
| | VL | 89 | CDR1: TITCRASQGISSWLA |
| | | 90 | CDR2: AASSLQS |
| | | 91 | CDR3: QQYNSYPLT |

### Isolated Nucleic Acid Sequence, Vector, and Cells

Further provided herein is an isolated nucleic acid sequence encoding any one of the recombinant polypeptides as disclosed herein.

In some examples, the peptide comprises an scFv encoded nucleotide having a sequence with at least 60% (e.g., at least 65%, at least 70%, at least 75% at least 80%, at least 85%, at least 90%, at least 95%, 100%) identity to SEQ ID NOS: 39-44. In some examples, the peptide comprises an scFv encoded nucleotide having a sequence with at least 60% (e.g., at least 65%, at least 70%, at least 75%, at least 80%, at least 85%, at least 90%, at least 95%, 100%) identity to SEQ ID NO: 39.

In further examples, the peptide comprises an scFv encoded nucleotide having a sequence with at least 60% (e.g., at least 65%, at least 70%, at least 75%, at least 80%, at least 85%, at least 90%, at least 95%, 100%) identity to SEQ ID NOS: 45-49. In some examples, the peptide comprises an scFv encoded nucleotide having a sequence with at least 60% (e.g., at least 65%, at least 70%, at least 75%, at least 80%, at least 85%, at least 90%, at least 95%, 100%) identity to SEQ ID NO: 45.

In certain examples, the peptide comprises an scFv encoded nucleotide having a sequence with at least 60% (e.g., at least 65%, at least 70%, at least 75%, at least 80%, at least 85%, at least 90%, at least 95%, 100%) identity to SEQ ID NOS: 50-51. In some examples, the peptide comprises an scFv encoded nucleotide having a sequence with at least 60% (e.g., at least 65%, at least 70%, at least 75%, at least 80%, at least 85%, at least 90%, at least 95%, 100%) identity to SEQ ID NO: 50.

In further examples, the peptide comprises an scFv encoded nucleotide having a sequence with at least 60% (e.g., at least 65%, at least 70%, at least 75%, at least 80%, at least 85%, at least 90%, at least 95%, 100%) identity to a SEQ ID NO: of Table 5.

Also provided herein is a vector comprising the isolated nucleic acid sequence as disclosed herein.

Further provided herein is a cell comprising the vector as disclosed herein.

In some examples, the cell reduces tumor activity when the antigen binding domain of the CAR binds to CD74.

In further examples, the cell comprises a cell of Table 2.

TABLE 2
Cells in tumor microenvironment.
Cells in Tumor Microenvironment

| | |
|---|---|
| Innate Immune Cells | myeloid-derived suppressor cells (MDSCs) |
| | tumor-associated macrophages (TAMs) |
| | tumor-associated neutrophils (TANs) |
| | tumor-associated dendritic cells (tDCs) |
| | Eosinophils |
| | platelets |
| Adoptive Immune | regulatory T cells (Tregs) |
| Cells | CD8+ T cells |
| | CD4+ T cells |
| | mast cells |
| | regulatory B cells (Bregs) |
| Interface of | Regulatory NK cells |
| Adaptive and Innate | Invariant NKT cells |
| Immunity | Gamma delta T cells |
| | Innate-like lymphocytes |
| Stromal Cells and | Cancer-associated fibroblast |
| Metrix | Adipocytes |
| | Neurons and nerves |
| Vascular Cells | Blood vascular endothelial cells |
| | Lymphatic ECs |
| | Pericytes |

### Methods

### Method of Treating Lymphoma

The present disclosure, in one aspect, provides for a method of treating lymphoma in a subject in need thereof, the method comprising administering to the subject a therapeutically effective amount of the chimeric antigen receptor polypeptide as disclosed herein.

Lymphoma refers to disease characterized by blood cell tumors that develop from lymphatic cells. The tumors that develop can include CD74 tumors. In some examples, lymphoma can include Hodgkin's Lymphoma or non-Hodgkin's Lymphoma (NHL).

NHL can affect either of B lymphocytes or T lymphocytes, wherein B lymphocytes produce antibodies to combat bacteria and viruses, while T lymphocytes destroy germs or abnormal cells in the body and/or boost or slow the activity of other immune system cells.

In further examples, non-Hodgkin's lymphoma comprises mantle cell lymphoma (MCL).

MCL results from a malignant transformation of a B lymphocyte in the outer edge of a lymph node follicle, referred to as the “mantle zone”. The transformed B lymphocyte grows in an uncontrolled fashion, resulting in the accumulation of lymphoma cells, which causes enlargement of lymph nodes. MCL cells can enter the lymphatic channels and the blood, and can spread to other lymph nodes or tissues, such as the bone marrow, liver, and gastrointestinal tract. A diagnosis of MCL is made when lymphoma cells from a lymph node tissue biopsy are examined under a microscopic and have surface markers of B cells (e.g., CD20), overexpress the cyclin D1 protein within the cells, and contain the translocation 11; 14. Blood tests and body imaging scans can be performed to determine the extent of the disease. MCL can include a rare blastoid variant in which the cells are bigger, grow and divide more rapidly, more aggressively, and is increasingly challenging to treat in comparison to the more common types of MCL.

MCL comprises Stage I, Stage II, Stage III, or Stage IV disease.

Stage I MCL corresponds to when the disease is in one lymph node region or a single organ above the diaphragm. Stage II MCL corresponds to existence of the disease in two or more lymph node regions on the same side of the diaphragm. Stage III MCL corresponds to existence of the disease in two or more lymph node regions above and below the diaphragm. Stage IV corresponds to when the disease is widespread in lymph nodes and/or other parts of the body above and below the diaphragm.

In some examples, the method further comprises administering a CD74 expression modulation agent.

In some examples, the method further comprises administering a therapeutically effective amount of an autophagy inhibitor.

Autophagy is a self-degradation process in which damaged proteins and organelles are engulfed into autophagosomes for digestion and eventually recycled for cellular metabolism to maintain intracellular homeostasis. Once cancer has formed, autophagy can protect the cancer cells by providing extra nutrients to them or by keeping anticancer drugs or other substances from destroying them. Autophagy may also affect the body's immune response against viruses, bacteria, and cancer cells. Therefore, cancer treatments can include administering autophagy inhibitors as a means of therapeutically treating cancer. Autophagy inhibitors include, but are not limited to, chloroquine and hydroxychloroquine.

In further examples, the method further comprises administering a therapeutically effective amount of a histone deacetylase (HDAC) inhibitor.

HDAC inhibitors are a class of anti-cancer agents that play roles in epigenetic or non-epigenetic regulation, inducing death, apoptosis, and cell cycle arrest in cancer cells. HDAC inhibition can affect tumor cell survival by blocking tumor angiogenesis and by inhibiting intracellular stress response pathways. HDAC inhibitors can include, but are not limited to, vorinostat, depsipeptide, romidepsin, panobinostat, and belinostat.

In further examples, the chimeric antigen receptor is administered in combination with at least one cancer therapy.

In certain examples, the additional cancer therapy comprises surgery, chemotherapy, immunotherapy, ionizing radiation, or a combination thereof.

In specific examples, surgery can include stem cell transplantation. Stem cell transplantation (SCT), also referred to as a bone marrow transplant, is a procedure in which a subject receives healthy stem cells to replace damaged stem cells. This can include autologous transplantation in which the transplantation uses the subject's own stem cells. SCT can also include allogeneic transplantation, which uses stem cells from a donor.

In autologous transplantation, the subject stem cells are collected and stored. The cells are frozen and then returned to the subject after receiving intensive high-dose chemotherapy either with or without radiation therapy. This procedure can be used in clinically symptomatic subjects that are fit, young, and have few or no coexisting illnesses.

In some examples, chemotherapy can include R-CHOP (Rituxan, cyclophosphamide, doxorubicin, vincristine, and prednisone), VcR-CAP (bortezomib, rituximab, cyclophosphamide, doxorubicin, and prednisone), R-hyperCVAD (rituximab, cyclophosphamide, vincristine, doxorubicin, and dexamethasone alternating with high-dose cytarabine and methotrexate), B+R (bendamustine and rituximab), R-FCM (rituximab, fludarabine, cyclophosphamide, and mitoxantrone), R-DHAP (rituximab, dexamethasone, cytarabine and cisplatin), R-CVP (rituximab, cyclophosphamide, vincristine, and prednisone), or R-CBP (rituximab, cyclophosphamide, bortezomib, and prednisone), or any combination thereof. These chemotherapies can be administered intravenously or via mouth.

In further examples, cancer therapy can include R-CHOP followed by an autologous SCT, R-CHOP followed by higher doses of cytarabine and further followed by an autologous stem cell transplant, or R-hyperCVAD with autologous SCT, or any combination thereof.

### Method of Reducing Tumor Activity

The present disclosure, in one aspect, provides for a method of reducing tumor activity in a subject with lymphoma, the method comprising administering to the subject a therapeutically effective amount of the chimeric antigen receptor polypeptide as disclosed herein.

In further examples, non-Hodgkin's lymphoma comprises mantle cell lymphoma (MCL).

In certain examples, the mantle cell lymphoma comprises Stage I, Stage II, Stage III, or Stage IV disease.

In some examples, the method further comprises administering a CD74 expression modulation agent.

In some examples, the method further comprises administering a therapeutically effective amount of an autophagy inhibitor.

Autophagy is a self-degradation process in which damaged proteins and organelles are engulfed into autophagosomes for digestion and eventually recycled for cellular metabolism to maintain intracellular homeostasis. Once cancer has formed, autophagy can protect the cancer cells by providing extra nutrients to them or by keeping anticancer drugs or other substances from destroying them. Autophagy may also affect the body's immune response against viruses, bacteria, and cancer cells. Therefore, cancer treatments can include administering autophagy inhibitors as a means of therapeutically treating cancer. Autophagy inhibitors include, but are not limited to, chloroquine and hydroxychloroquine.

In further examples, the method further comprises administering a therapeutically effective amount of a histone deacetylase (HDAC) inhibitor.

HDAC inhibitors are a class of anti-cancer agents that play roles in epigenetic or non-pigenetic regulation, inducing death, apoptosis, and cell cycle arrest in cancer cells. HDAC inhibition can affect tumor cell survival by blocking tumor angiogenesis and by inhibiting intracellular stress response pathways. HDAC inhibitors can include, but are not limited to, vorinostat, depsipeptide, romidepsin, panobinostat, and belinostat.

In further examples, the chimeric antigen receptor is administered in combination with at least one cancer therapy.

In certain examples, the additional cancer therapy comprises surgery, chemotherapy, immunotherapy, ionizing radiation, or a combination thereof.

In further examples, the present disclosure, in one aspect, provides for a method of reducing tumor activity in a subject with classical Hodgkin's lymphoma, T cell lymphoma, or a combination thereof, the method comprising administering to the subject a therapeutically effective amount of the chimeric antigen receptor polypeptide as disclosed herein.

In some examples, the present disclosure, in one aspect, provides for a method of treating classical Hodgkin's lymphoma, T cell lymphoma, or a combination thereof, in a subject in need thereof, the method comprising administering to the subject a therapeutically effective amount of the chimeric antigen receptor polypeptide as disclosed herein.

In certain examples, the chimeric antigen receptor polypeptide administered to a subject targets immunosuppressive lymphoma microenvironment (LME). Immunosuppressive cells are part of the LME and promote tumor growth. Since these immunosuppressive cells within the LME express CD74, in some examples, the CAR polypeptides disclosed herein are used to target them.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: A Novel CAR-T Therapy Targeting CD74 and Offers Durable Response in Mantle Cell Lymphoma

Mantle cell lymphoma (MCL) is an incurable non-Hodgkin lymphoma with an estimated 5,000 new cases diagnosed each year in the United States, signifying the need for new therapeutic approaches. Milatuzumab is an IgG1 monoclonal antibody targeting CD74, however, the clinical efficacy is minimal due to the rapid internalization and short half-life. It was hypothesized that targeting CD74 using a chimeric antigen receptor T cell in MCL to engage CD74 overexpressed MCL cells could present potent and durable anti-tumor activity.

A second generation anti-CD74 CAR (74bbz) with 4-1BB and CD3z signaling domains was engineered. Through in-silico and random mutagenesis on the scFV domains, the 74bbz CAR was functionally optimized with superior antigen binding, proliferation, and cytotoxicity when encountering MCL cells in vitro and in vivo.

Clone 42105 74bbz (42105-74bbz) CAR-T cells exerted specific cytolysis on a panel of MCL cell lines and primary patient samples, but not CD74 negative cell line SUDHL-1. CD74 ubiquitously restricted to express mainly on CD33+ immune populations from healthy donors while it expressed minimally on lymphocytes except B cells. More importantly, the relatively lower level of CD74 on normal immune cells did not induce significantly specific lysis in vitro and in vivo. Using an NOD-SCIDγc^{-/-} (NSG) mouse model with lymphoma cells from an aggressive CD19 CAR-T therapy relapsed patient, the absolute number of persistent CAR-T cells were higher in 42105-74bbz CAR-T-treated mice than those with parent-74bbz CAR-T. In a subcutaneous MCL NSG xenograft model, survival was significantly prolonged using more persisting CAR-T cells in 42105-74bbz CAR-T treated mice and comparable to CD19bbz CAR-T treated mice.

CD74 on MCL was better targeted by 74bbz CAR-T therapy and 42105-74bbz CAR-T cells could provide a durable and potent anti-tumor activity for improved clearance of MCL.

To overcome the limitations of milatuzumab, an anti-CD74 CAR-T cell product named 42105-74bbz with 4-1BB+CD3ζ as co-stimulatory domains was developed and functionally optimized, and its activity investigated in preclinical MCL models. It was hypothesized that anti-CD74 CAR-T cell therapy would provide a safe and long-lasting anti-lymphoma effect. Findings showed that 42105-74bbz CAR-T cells exhibited cytotoxicity against both MCL cell lines and primary MCL patient samples and their activity positively correlated with target antigen density. In vivo, 42105-74bbz CAR-T cells prolonged the survival of a patient derived xenograft (PDX) mouse model established from a MCL patient cells that had relapsed following CD19 CAR-T therapy, as well as in a subcutaneous human cell line xenograft MCL model. This approach was not inferior to CD19 CAR-T cell therapy in vivo. Furthermore, no significant changes were observed in the absolute cell count of B cells, monocytes, myeloid suppressor cells, and NK cells following treatment with 42105-74bbz CAR-T cells in a humanized mice using CD34+ stem cells.

### Methods

### CD74 CAR-T Vector Construction and Expression

A second-generation CAR was designed, where single chain variable fragments (scFv) of the mouse anti-human CD74 was joined with 4-1BB and CD3ζ chain via the CD8 hinge region and transmembrane domain. A CMV promoter was added in front of the entire sequence for constitutively expression. The sequence was then codon-optimized, synthesized (Twist Bioscience, OR), and subcloned into a lentiviral vector pCDH (SBI Bioscience, CA). Lentiviral virus was made by transfecting Lenti-X 293T cells (Clontech, Takara Bio USA Inc, CA) with the CAR construct, packaging plasmids including PsPax (Addgene #12260) and pMD2.G (Addgene #12259) via Lipofetamine 2000 (Invitrogen, MA). 48 hours post-transfection, the viral supernatant was harvested, filtered against 0.22 um and then snap-frozen at −80° C. until it was used.

In Silico scFv Optimization and Mutagenesis

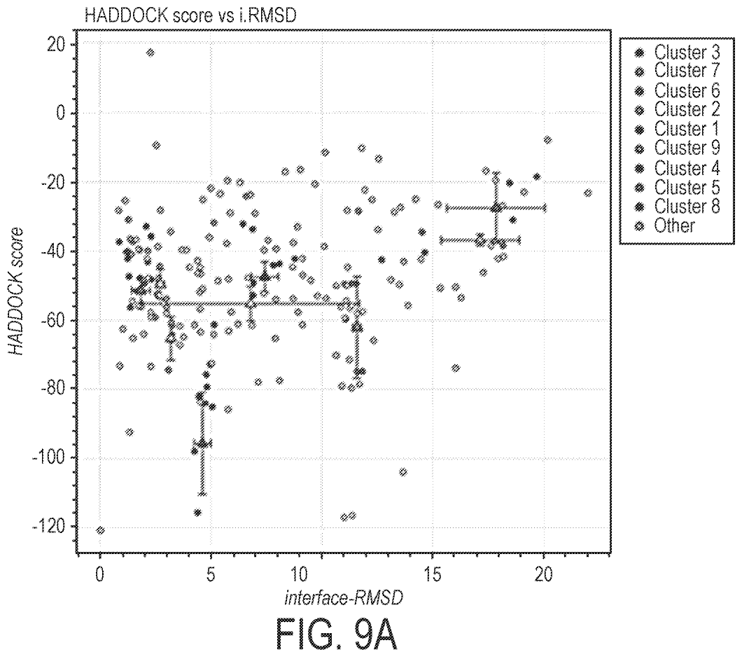

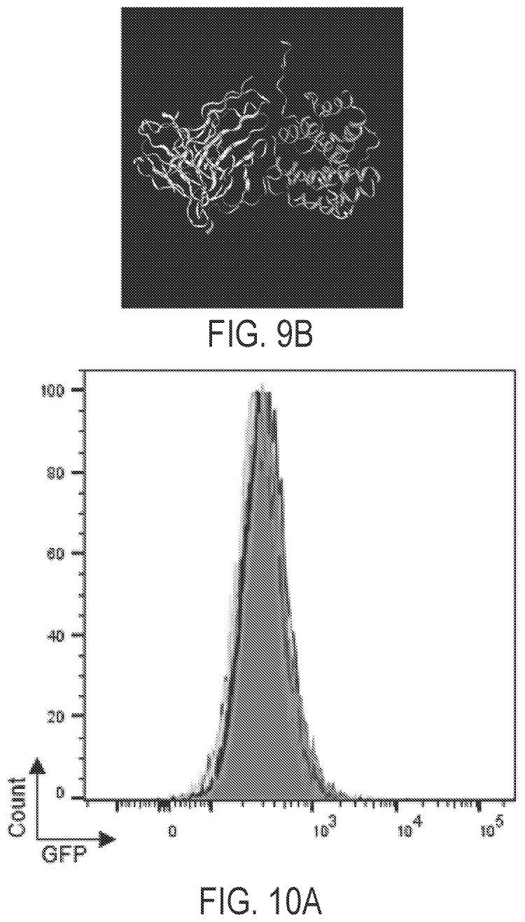

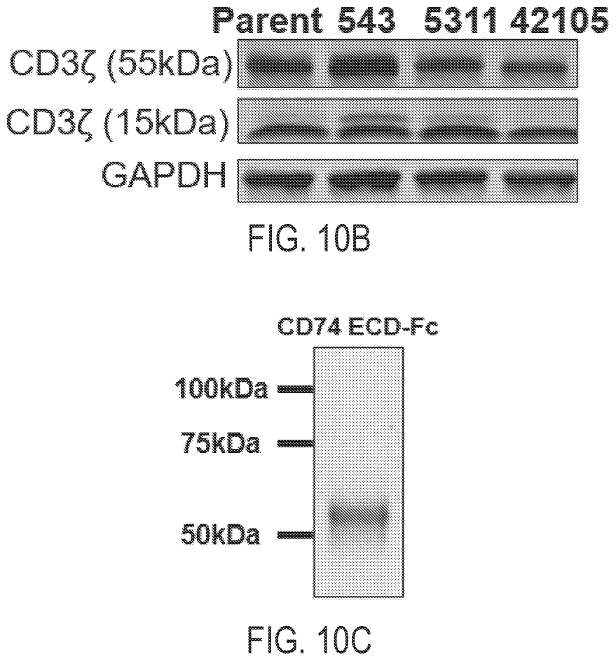

Amino acid paratope in the regions of complementarity-determining region (CDR)1, CDR2 and CDR3 was first identified by ProABC-2. The scFV was then in silico reconstructed as PDB by ABodyBuilder2 (Leem, 2019) and prepared for antigen (CD74, PDB #1IIE). The scFV was then docked to the antigen using HADDOCK 2.4. (Ambrosetti, 2023) To refine the docking model, alanine scanning on the amino acid residues on each CDR was performed. Among all model cluster candidates, the best with the lowest HADDOCK score was picked for computational mutagenesis study (FIGS. 9A-9B). The binding residue candidates were substituted and docked against CD74 antigen with all the possible 20 amino acids. By using Eris molecular suite, (Yin, 2007) mutation was introduced in the scFV regions on those binding residues. The estimated free energies of mutant conformations were compared with the wild-type and lead mutants were selected for site-directed mutagenesis and downstream functional assays. Mutagenesis on the CDR regions were performed by GeneMorph II EZClone domain mutagenesis kit (Agilent, CA) according to the manufacturer's instructions. 3 CAR constructs with anti-CD74 scFV (named 543, 553, and 563) from public domains were also developed and used for comparison. The mutant plasmid was transformed, and lentiviral production was performed on each CAR mutant. To facilitate the screening, each mutant was displayed on T-ALL cell line Jurkat cell, and the expression level of the CAR was uniquified by sorting the GFP+ cells from each mutant by flow cytometry and CD3ζ by immunoblot (FIGS. 10A-10B). The mutant performance was assessed and compared with the parent 74bbz CAR. by 4 parameters: 1) functional binding affinity to a chimeric CD74 extracellular domain (ECD)-Fc fusion protein (FIG. 10C); 2) CD69 activation marker expression upon engaging CD74+ target cells; 3) repeated antigen proliferation assay; and 4) in vitro cytotoxicity assay against CD74+ target Mino cells.

### Cell Culture and Isolation

MCL cell lines JeKo-1, Mino, Sp53, UPN-1, Granta-519, and Z-138 were obtained from ATCC (Manassas, VA) and cultured under the manufacturer's instruction. All the cell lines used were routinely tested for mycoplasma with MycoAlert (Lonza, MA) and passaged for no more than two months. Human peripheral blood mononuclear cells (PBMCs) were isolated from healthy blood donors by Ficoll-Paque Plus (GE Healthcare Life Science, PA) gradient density with the approval of the Institutional Review Board at The Ohio State University. Human T cells from peripheral blood were isolated using CD4 and CD8 microbeads in a ratio of 1:1 following the manufacturer's instructions (Miltenyi Biotech, CA). To determine if activations on T cell and B cell induce CD74 upregulation, T cell were treated overnight with CD3 and CD28 soluble antibody (10 ng/mL, BioLegend, CA) and 250 U/mL IL-2. B cells were isolated from PBMCs using Easysep human B cell isolation kit (StemCell Technologies, MA) while B cells were treated with LPS (10 ng/mL) and anti-IgM antibody (10 ug/mL). The activation status of the cells was confirmed by flow cytometry.

### Antibodies and Flow Cytometry

Antibodies used in this study include anti-CD3 (clone SK7 and 145-2C11), CD56 (clone N901), CD14 (clone MφP9), human CD45 (clone 2D1), mouse CD45 (clone 30-F11), human NKG2D (clone 1D11), CD4 (clone RPA-T4), CD69 (clone FN50), CD25 (clone BC96). All antibodies are from BioLegend, CA, except CD74 (clone MB741) was from BD Biosciences, CA. Cells were washed once with PBS, first blocked with Trustain human Fc blocker (BioLegend) stained with antibodies for 20 min at room temperature and analyzed with a LSRII flow cytometer (BD Biosciences, CA, USA). For parent or mutant CAR-T detection on primary T cells, a truncated EGFR (tEGFR) tag was used in the CAR construct and detected by anti-EGFR antibody (AY13, BioLegend). For CD74 antigen density determination, a calibration curve correlating instrument detection channel values and standardized fluorescence intensity units on Molecules of Soluble Fluorochrome (MESF) FITC-5 premix beads (Bangs Laboratory Inc, IN) was constructed with R² of 0.9995 and the equation was used to calculate the antigen density of CD74 on MCL cells from the mean fluorescence intensity obtained on the same day and settings according to the manufacturer's instruction. For flow cytometry based functional binding affinity assay, it was modified to measure the CD74 scFV on Jurkat cells to CD74 extracellular domain (ECD)-Fc. Briefly, 1×10⁶ Jurkat cells (viability >90%) carrying parent or mutant scFV were washed twice with cold PBS and blocked with Fc blocker (Trustain human Fc blocker, BioLegend) for 10 min at room temperature. The cells were stained with 5 μg/mL CD74-ECD (Sino Biological US Inc., PA) in excess for 45 min on ice, then washed twice with PBS before stained with anti-Fc flow cytometric grade antibody and SYTOX Blue dead cell stain (Invitrogen) for 15 min at room temperature. The cells were immediately analyzed by flow cytometry after being washed twice with cold PBS. To confirm the activation of Jurkat cells or primary T cells, the expressions of CD69 and CD25 were determined by flow cytometry.

### Repeated Antigen Stimulation Assay

The stimulation and proliferation methods were performed as previously reported with modifications. (Smith, 2018) Jurkat cell or primary T cell clones were co-cultured with irradiated Mino cells (stimulator) at effector-to-target ratio (ET ratio) of 1:2 at a total cell density of 3×10⁵/mL with RPMI 1640 medium containing reduced FBS at 1%. The culture was refreshed with medium every three days and restimulated with freshly irradiated Mino cells for a total of three times before the absolute cell counts were determined by trypan blue exclusion assay. Parent CD74bbz CAR and untransduced-Jurkat or T cells with or without stimulators were used as negative control.

### Cytotoxicity Assay

The cytotoxicity of the CAR-T cells was performed with ToxiLight™ non-destructive cytotoxicity bioassay kit (Lonza) and as manufacturer's instruction described (29). Briefly, the MCL cell lines or primary MCL patient samples (lymphoma % ranged from 73.7% to 98.7%) were co-cultured with 74bbz CAR-T cells or untransduced T cell control (UTT) cells at ET ratio of 5:1 for 24 hours. At 24 h, the wells for maximum lysis were added with 100% Lysis Buffer (Lonza) for 10 min at room temperature. The volume in other wells were adjusted with the provided Tris AC buffer. The cell supernatant from each well was then harvested and reacted with the provided substrate for 5 min before the plates were read for bioluminescence with a Synergy HT microplate reader (Biotek, VT).

### ELISA

Measurement of human IFN-γ in the culture supernatant was performed with ELISA MAX™ Deluxe Set human IFN-γ kit (BioLegend) according to manufacturer's instruction. Cell-free supernatant was harvested from cell culture 24 hours after coculture with effectors. The plate was then washed, incubated with tetramethylbenzidine substrate (Agilent Technologies, CA) and read at 450 nm using a Synergy HT microplate reader (Biotek, VT).

### In Vivo Experiment

All the animal studies were approved by The Ohio State University Institutional Animal Care and Use Committee. NOD-SCID^{IL2}γ^{-/-} (NSG) mice of 4- to 6-weeks old mice were used to establish a human PDX using MCL cells obtained from a patient that relapsed following CD19 CAR-T therapy. The PDX was developed from four rounds of serial adoptive transfer. The PDX MCL cells were cryopreserved and banked for future use. When used, 1×10⁶ PDX MCL cells were injected intravenously. After three days, the mice were randomized into groups as indicated and treated with 5×10⁶ 74bbz CAR-T cells, either the parent or mutant were injected. The progression of the disease was monitored by flow cytometry weekly. For a survival study, a subcutaneous tumor model using CD74+ Mino cells was also adopted. On day −1, 1×10⁶ Mino cells were subcutaneously (s.c.) injected. After 3 days when tumor was palpable, 5×10⁶ 74bbz CAR-T cells or UTT cell control were injected intratumorally. All the human T cells used are allogeneic to the Mino cells. The progression of the disease was monitored, and survival data were recorded. The mice were sacrificed when they reached an endpoint at body condition score <2, paralysis, and 20% body weight loss. To determine the activity of 74bbz CAR-T cells on human immune cell subsets in vivo, a humanized mice from human umbilical cord blood as previously described, was developed. (Verma, 2020) Briefly, 4-week-old female NSG mice were irradiated at 125 cGy (RS-2000, Rad Source Technologies, GA) and then intravenously injected with 5×10⁵ human CD34+ cells isolated by human CD34 microbead ultra-pure kit (Miltenyi Biotech) per mouse. The purities of the human CD34+ cells were >85% (n=3). The mice were intraperitoneally injected with SCF, GM-CSF, and IL-3 at 2 to 4 ng/mL for 3 weeks every other day to boost the development of myeloid lineage. After 10-week post-transplant of the human CD34+ cells, human chimerism in circulating cells were determined by facial/submandibular venous blood sampling and flow cytometry. The baseline absolute numbers of human immune cell subsets were collected, and the mice were randomized into groups received 5×10⁵ UTT or 5×10⁵ 42105-74bbz CAR-T cells. The absolute number of human B cell (human CD45+CD33− CD19+), monocyte (human CD45+CD11b+CD33+CD14+ cells), granulocytic myeloid suppressor cell (G-MDSC, human CD45+CD11b+CD33+CD14−HLA−DR−), monocytic myeloid suppressor cell (M-MDSC, human CD45+CD11b+CD33+CD14+HLA−DR−) and NK cell (human CD45+CD33−CD3−CD56+) were measured on Day 3, 11, 18, 23 post UTT/CAR-T injections. The UTT and CAR-T cells were traced and identified as human CD45+CD3+ and CD45+CD3+EGFR+, respectively.

### Statistical Analysis

For data following normal distribution, or normal after transformation, student t test or unpaired t-test was used to compare two independent groups or two matched groups. Linear model was used to compare multiple independent groups. Linear mixed model was applied to multiple group comparisons under the variance-covariance structure due to repeated measures. For survival data, Kaplan-Meier method was applied to estimate survival function and log rank test was used to compare the survival between two groups. P values were adjusted for multiple comparisons by Holm's procedure. A P value of 0.05 or less was considered statistically significant.

### Results

scFV Optimization of CD74Bbz CAR Identifies a Close of CAR-T Cells with Maximum Cytotoxicity and Target Induced Proliferation

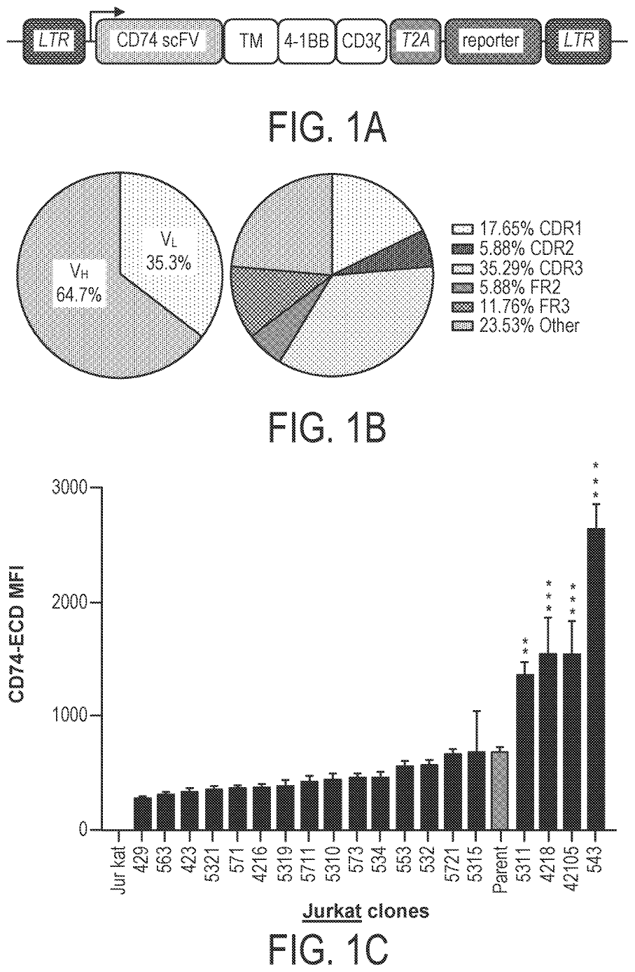

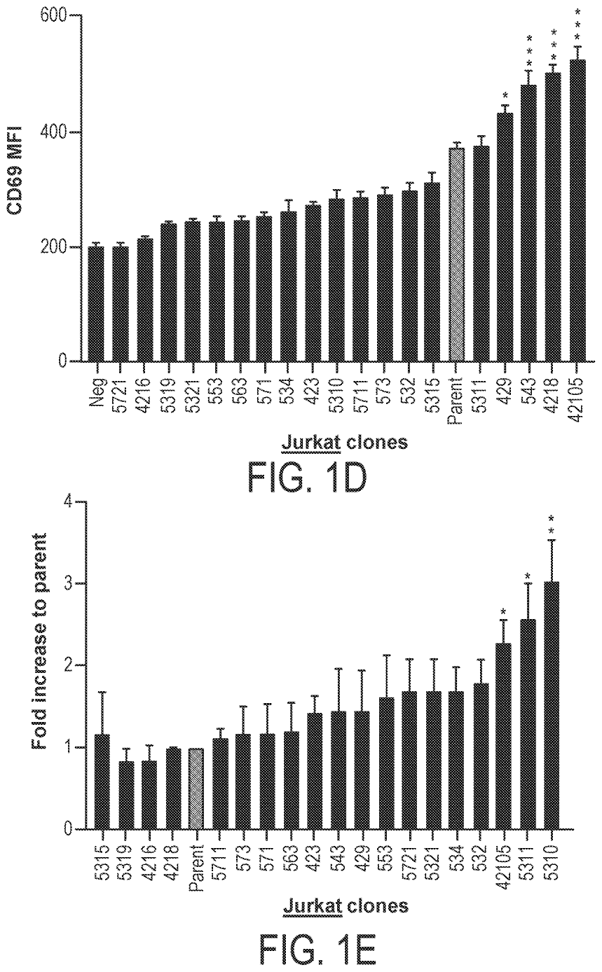

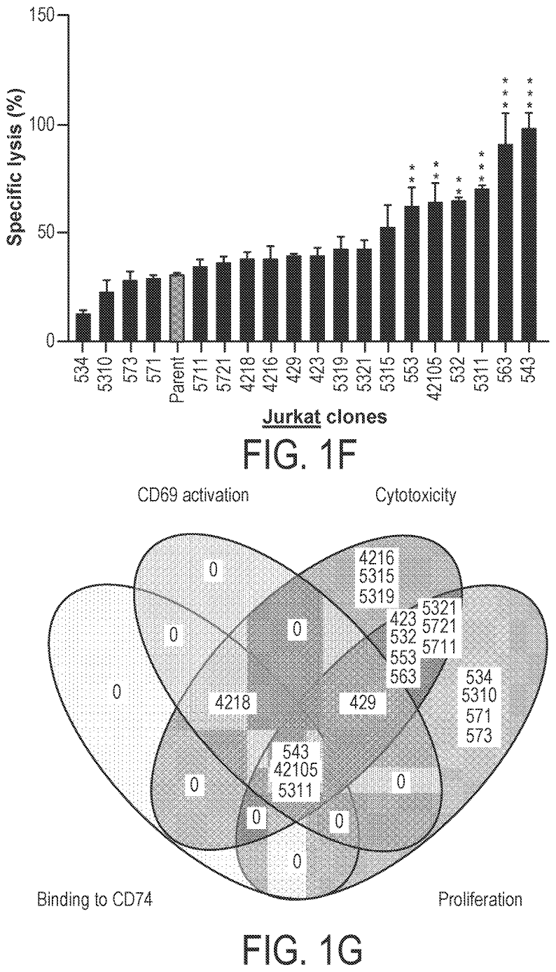

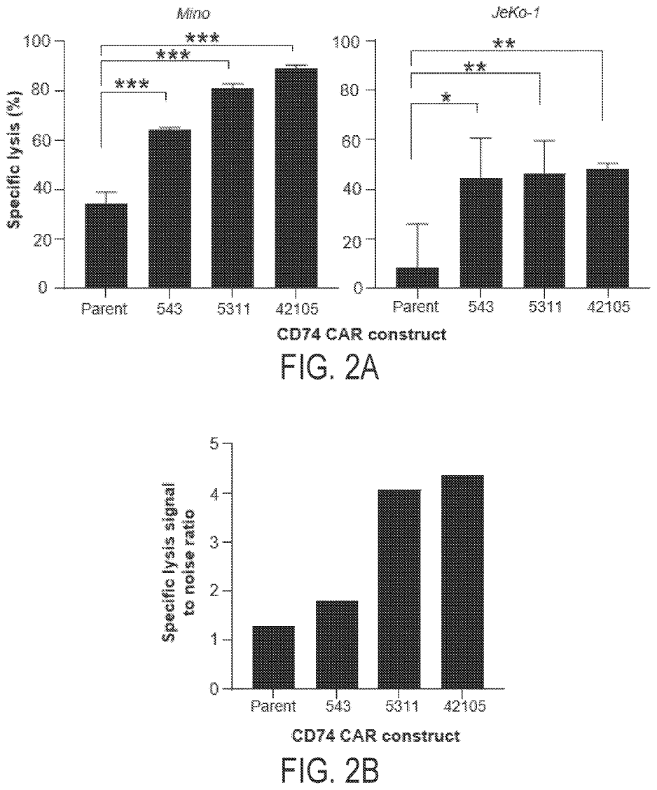

The second-generation CAR construct with 4-1BB and CD3ζ chain signaling domain was designed by joining a scFV specifically targeting human CD74 with the CD8 hinge/transmembrane domain and CD3ζ (FIG. 1A). An optimization of the CDR region of scFV was performed and aimed to select the best functional CD74bbz CAR-T cells by both an in-silico approach and functional assays. An anti-CD74 scFV-CD74 antigen docking model was created and refined by alanine scanning (FIGS. 9A-9C). For V_{H}, 4 positions in CDR1, 10 in CDR2, and 8 in CDR3 were found to be useful for the interaction. For V_{L}, 5 positions in CDR1, 2 in CDR2, and 6 in CDR3 were the important interacting residues. The 22 amino residues were then mutated in the V_{H} and 13 in the V_{L} using mCSM-AB2, a computational application to rationally assess the impact of single-point mutations on the binding affinity between antibodies and antigens with high accuracy. (Myung, 2020) A total of 741 mutants were successfully tested and the potential energy difference upon mutation between the wild-type and the mutants were ranked in ΔΔG (kcal/mol). A total of 17 mutants were chosen based on the positive energies gained from the wild type. 64.7% of the 17 mutants were on V_{H} while 35.3% were on V_{L} (FIG. 1). In addition, 35.3% of the 17 mutans were on CDR3 regions, 17.65% in CDR1, and 5.86% in CDR2 (FIG. 1). A T-acute lymphoid leukemia cell line (Jurkat) was engineered to express the selected mutants. The expression of CAR on each mutant clone was normalized by sorting each Jurkat clone at the same intensity of the GFP and confirmed by immunoblotting (FIGS. 10A-10B). The binding affinity of each mutant CAR to a chimeric CD74 ECD-Fc by flow cytometry (Geuijen, 2005) and 4 clones (5311, 4218, 42105, and 543) were found to have increased binding affinity to the CD74 antigen compared to the parent (FIG. 1C). The expression of the T cell activation marker CD69 was evaluated and identified 5 clones (429, 4218, 5310, 5311, and 42105) with significantly increased CD69 expression 8 hours after co-incubation with the MCL cell line Mino cells at an ET ratio of 1:2 (FIG. 1D). Following the repeated antigen stimulation assay, clones 42105, 5311, and 5310 displayed a significantly higher fold increase in cell numbers compared to the parent CAR (FIG. 1E). Similarly, clones 543, 563, 5311, 532, 42105 and 553 induced a significantly higher specific lysis compared to parent CAR-T cells (FIG. 1F). By including all clones with augmented functions than parent CAR-T cells in a Venn analysis, clones 42105, 5311, and 543 were identified as the three lead candidates for further screening as they met all the 4 proposed criteria CD69 activation, binding affinity, proliferation, and cytotoxicity (FIG. 1G). Using primary T cells (n=3) as effectors and MCL cell lines Mino (CD74 high expressor) and JeKo-1 (CD74 low expressor) as target cells, all 3 mutants induced significantly higher specific lysis compared to the parent 74bbz CAR-T cells (Mino, FIG. 2A; JeKo-1, FIG. 2A). To determine if the mutant clones were specific to CD74, the specific lysis on CD74 positive Mino cells (on-target signal) over the CD74 negative SUDHL-1 cells (off-target noise) was measured and compared among the 3 mutant clones over the parent. 5311 and 42105 showed higher signal to noise ratio than 543 (FIG. 2B).

### CD74+ MCL Cells are Sensitive to 42105-74Bbz CAR-T Cells

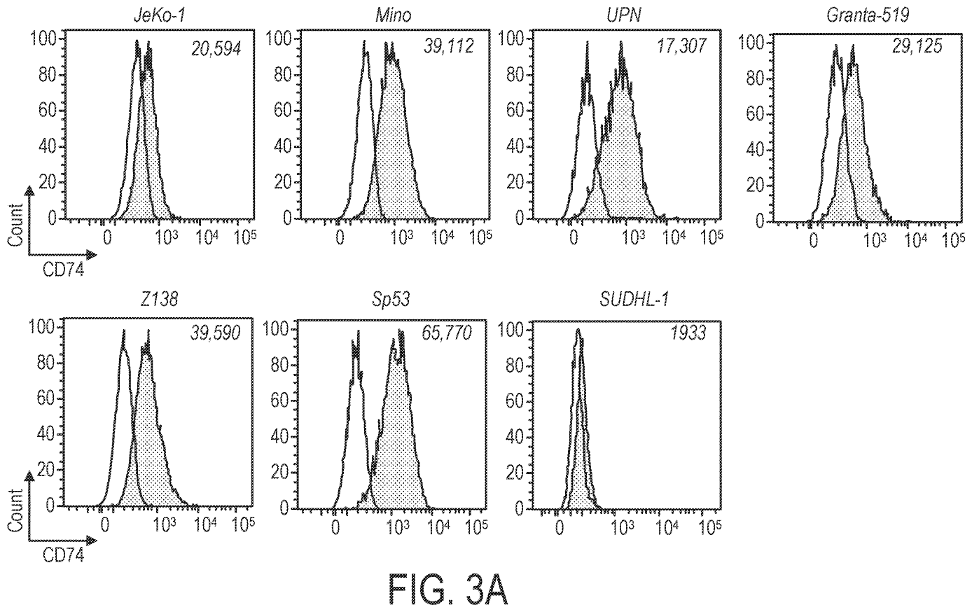

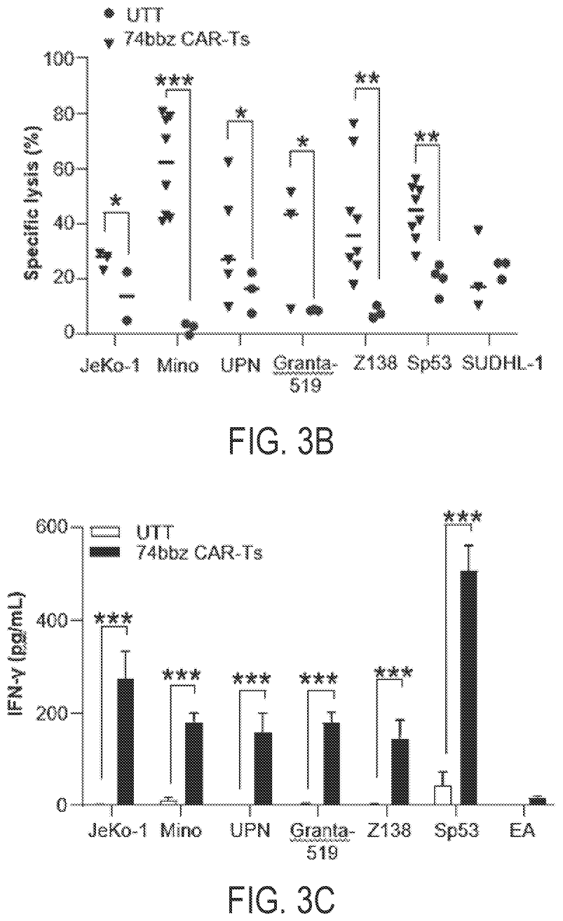

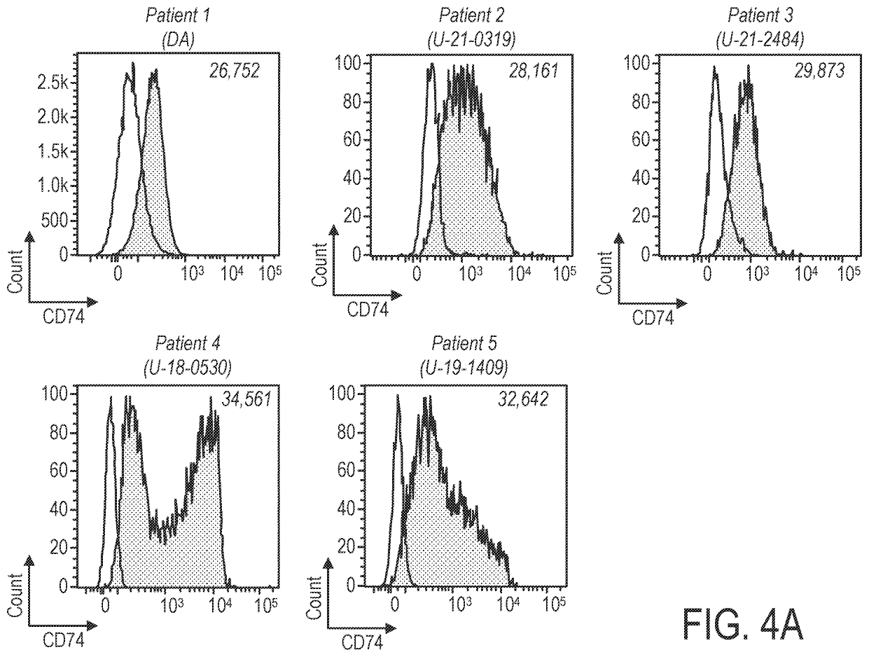

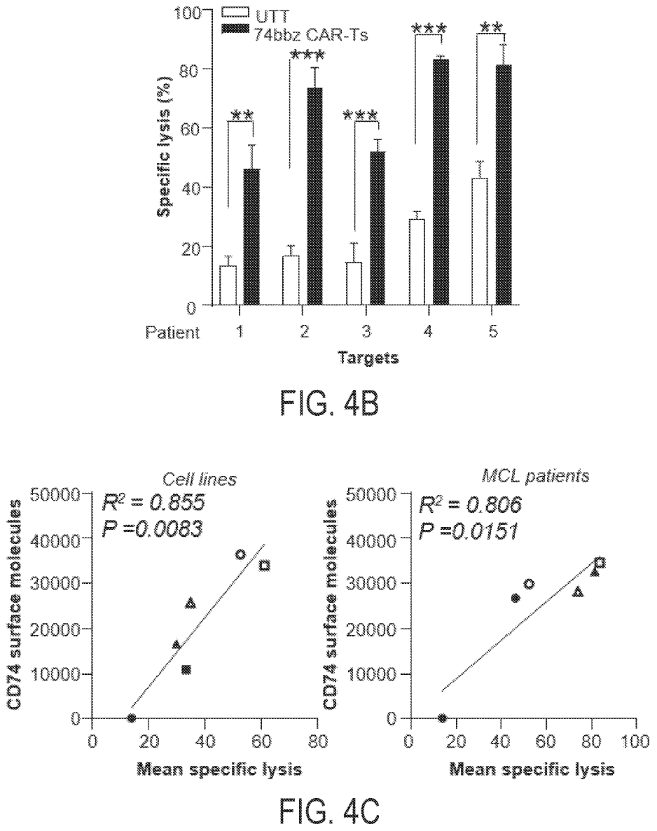

To test the sensitivity of MCL cells to 42105-74bbz CAR-T cells, cytotoxicity assays were performed in 6 CD74 positive MCL cell lines (JeKo-1, Mino, UPN-1, Granta-519, Z138, and Sp53) (FIG. 3A). 42105-74bbz CAR-T cells were generated using purified T cells from healthy donors with an average transduction efficiency at 3 days of 73.03±11.4%. The MCL cell lines were susceptible to the 42105-74bbz CAR-T cells with significant specific lyses compared to UTT control. A median of 28.1%, 62.4%, 43.3%, 43.6%, 35.8%, 45% of JeKo-1, Mino, UPN, Granta-519, Z138, and Sp53, respectively, were lysed at 24 hours with an ET ratio of 5:1 (FIG. 3B). Engaging 42105-74bbz CAR-T cells with the indicated target MCL cell lines induced T cell activation with a significant increase in IFN-γ production compared to the UTT and effector T cell controls (FIG. 3C). Five primary CD74+/CD19+/CD5+ MCL patient samples were used for further validation (clinical characteristics of these 5 MCL patients are summarized in Table 3). (FIG. 4A)

TABLE 3
Clinical characteristics of MCL patients.

| Patient | | | Immuno- |
|---|---|---|---|
| ID | Diagnosis | Treatment | phenotype |
| 1 (DA) | MCL | Treatment naive | CD5+CD19+ |
| 2 (U-21- | MCL | Treatment naive | CD5+CD19+ |
| 0319) | | | |
| 3 (U-21- | MCL | R/R status post chemotherapy | CD5+CD19+ |
| 2484) | | including ASCT, targeted | |
| | | therapies including ibrutinib | |
| | | and venetoclax, and | |
| | | progressed after commercial | |
| | | CD19 CAR-Ts | |
| 4 (U-18- | MCL | Treatment naive | CD5+CD19+ |
| 0530) | | | |
| 5 (U-19- | MCL | R/R status post chemotherapy | CD5−CD19+ |
| 1409) | | and ibrutinib | |

Co-culture of 42105-74bbz CAR-T cells with the primary MCL patient cells (n=5) at ET ratio of 5:1 for 24 hours resulted in significant lysis compared to UTT control in all the samples tested (FIG. 4B). Further analyses on the MCL cell lines (FIG. 4C) and primary MCL patient samples (FIG. 4C) showed a positive correlation between target antigen expression and cytotoxicity (R² coefficient of 0.855 and 0.806, respectively). These results indicate that MCL are sensitive to the effector functions of 42105-74bbz CAR-T cells.

### 42105-74Bbz CAR-T Cells are Minimally Cytotoxic Against Normal Immune Cell Subsets

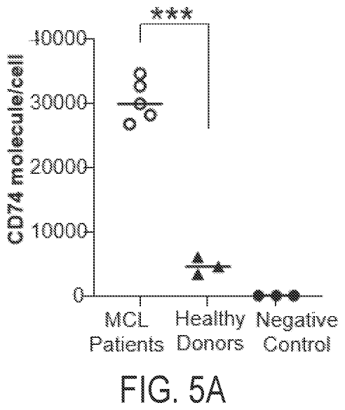

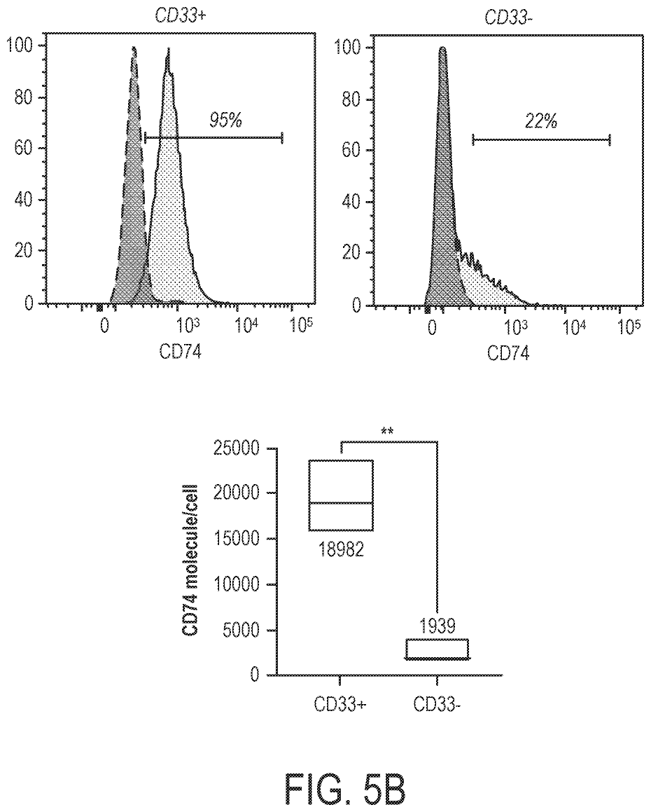

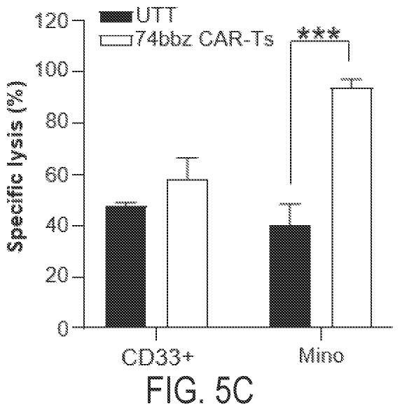

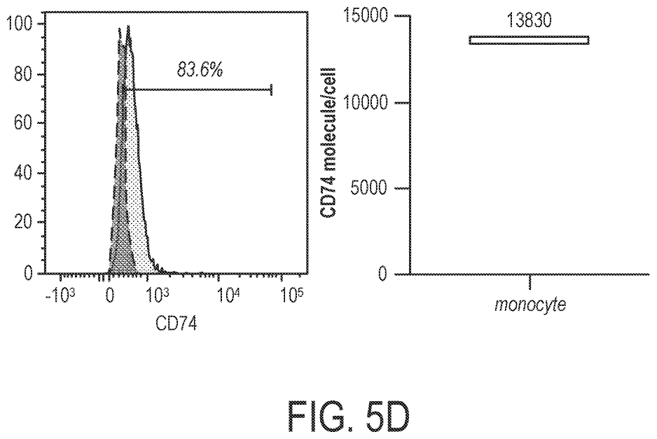

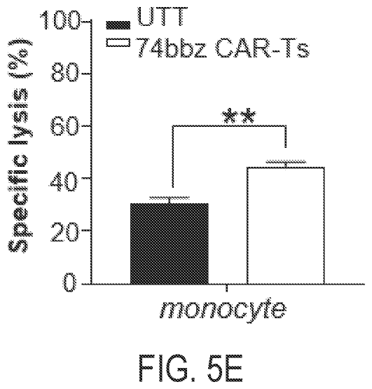

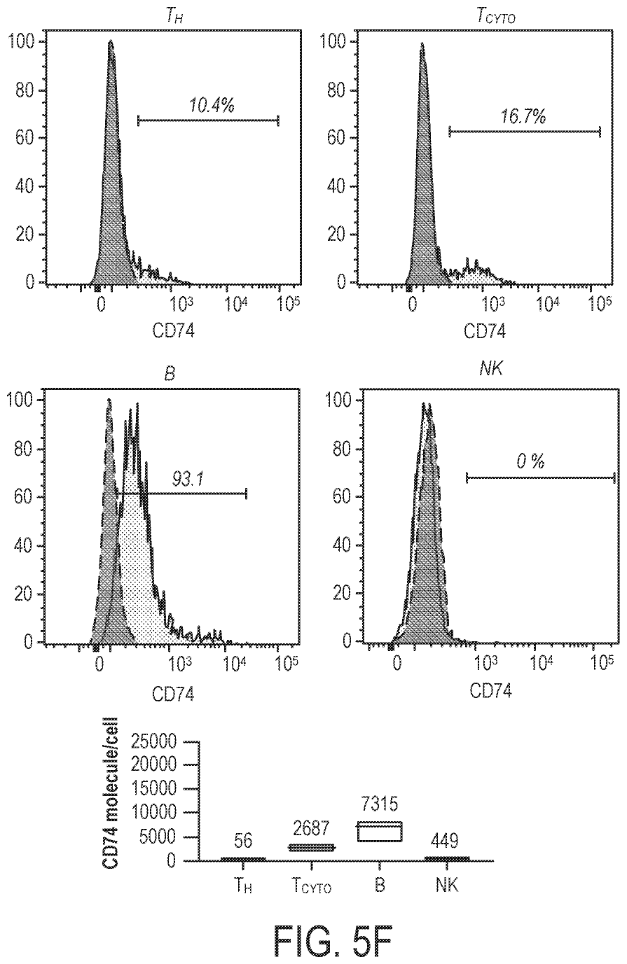

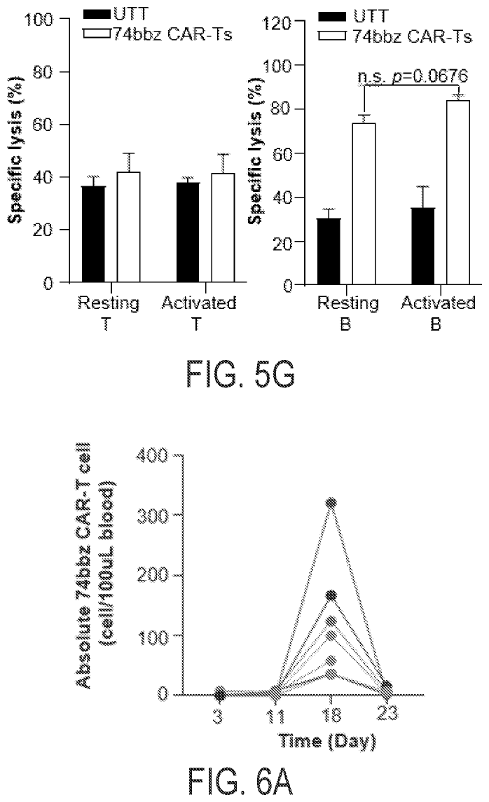

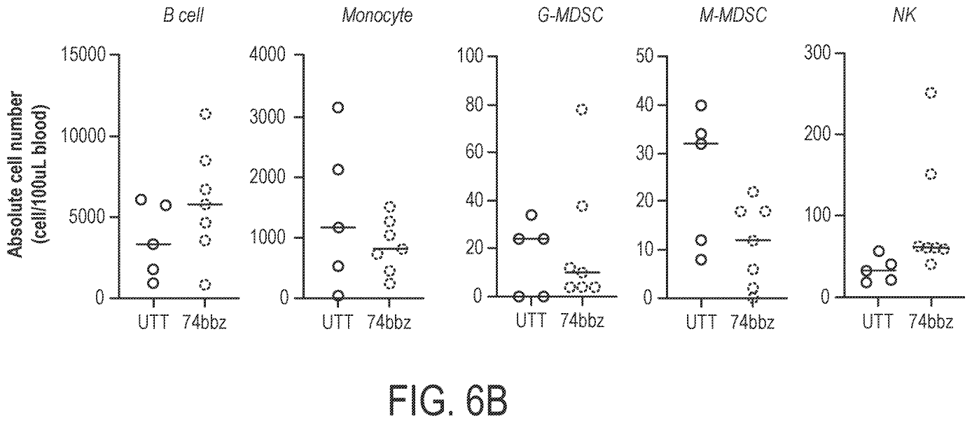

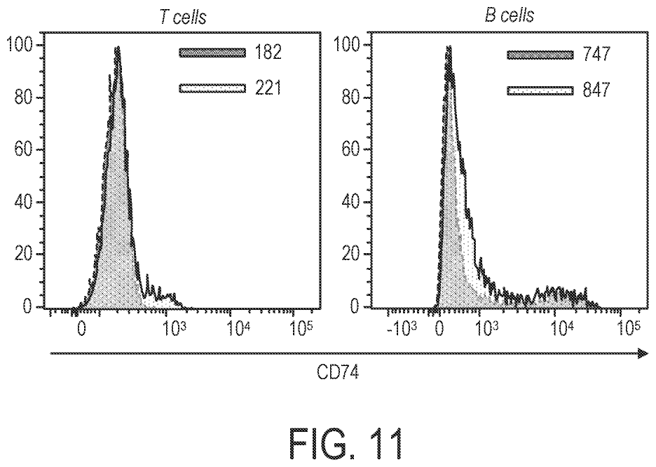

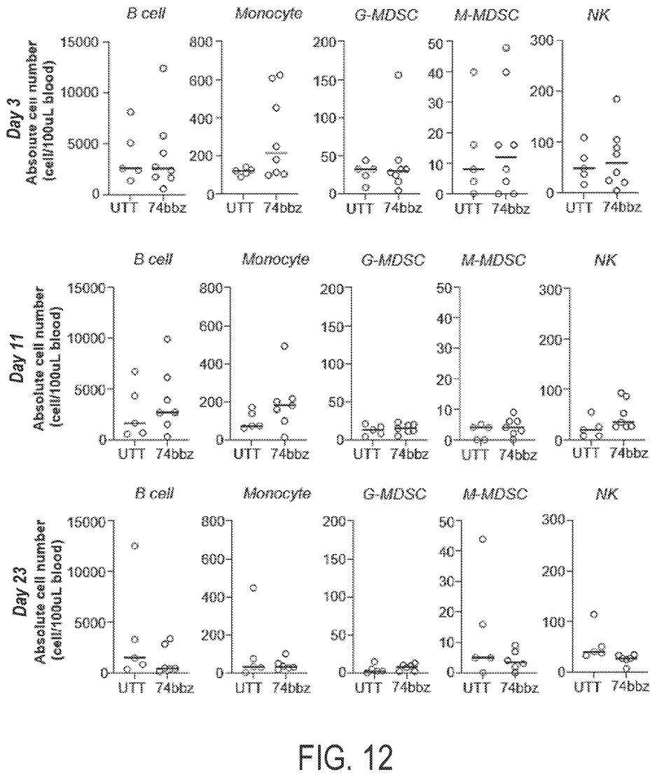

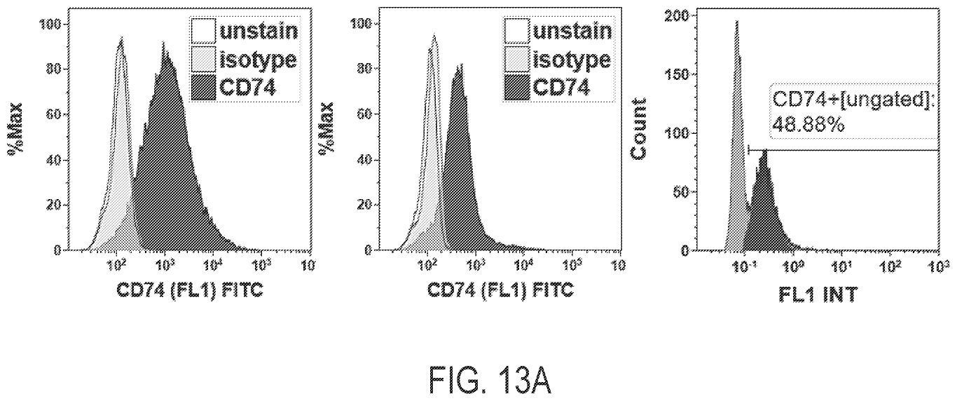

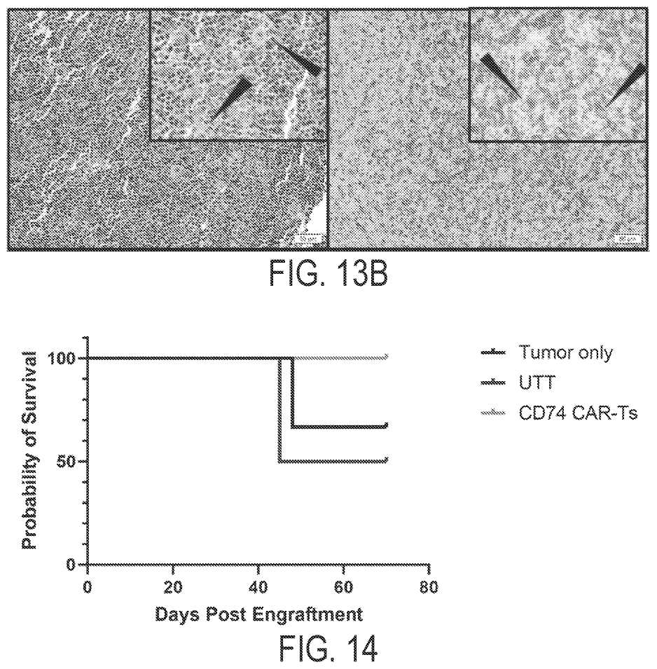

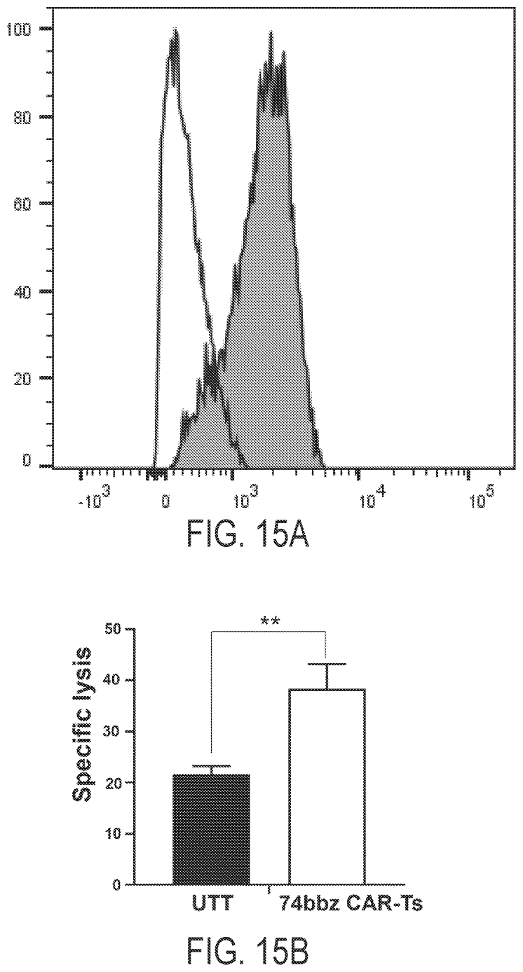

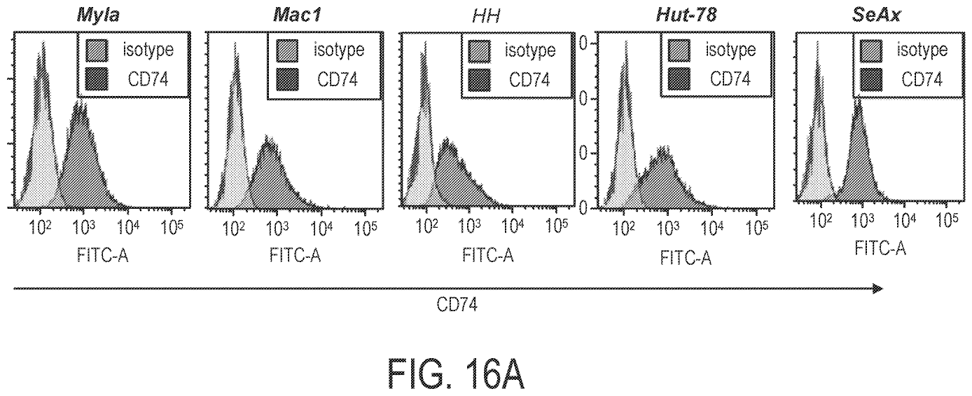

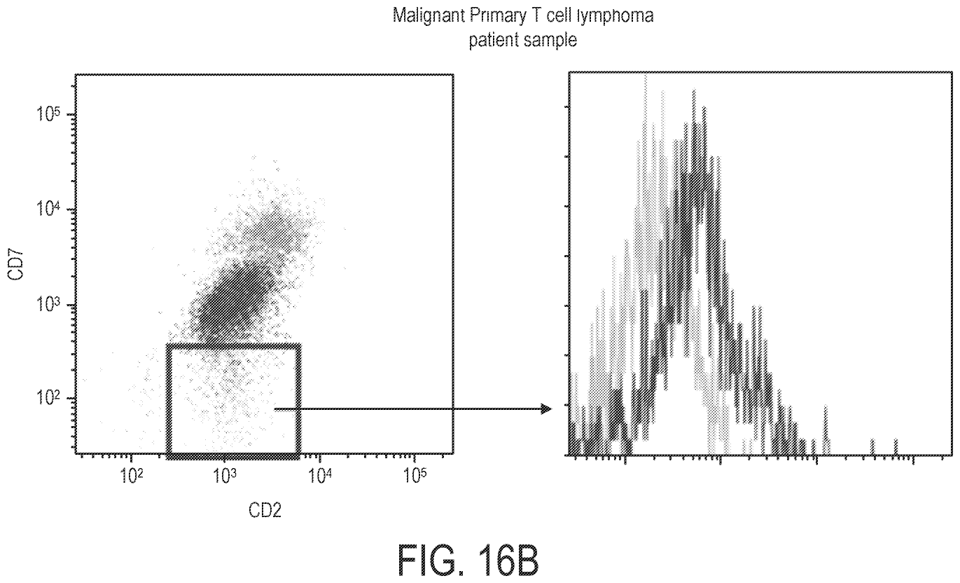

To differentiate the activity of 42105-74bbz CAR-T cells on MCL and non-malignant immune cells, CD74 density was measured in PBMCs from healthy blood donors (n=3) and primary MCL patient samples (n=5). PBMCs from healthy blood donors had less than 10,000 molecules per cell and significantly lower than the MCL cells which ranged from 26,752 to 34,561 molecules per cell (FIG. 5A, p=0.0019). There is no statistical difference between normal cells and negative control SUDHL1 cells (p=0.656). Within the PBMCs, CD33+ myeloid cells had higher percent positive and CD74 molecules per cell than the CD33− subset (95% vs 22% CD74+, FIG. 5B). 42105-74bbz CAR-T cells were minimally cytotoxic against CD33+ cells (FIG. 5C, p=0.456). Within CD33+ population, majority of monocytes (83.6%) were CD74+ and had a median of 13,830 CD74 molecules per cells (FIG. 5D). Using purified monocytes as target cells in the coculture of 42105-74bbz CAR-T cells, a significant increase in specific lysis from UTT was observed, indicating that they were susceptible to 42105-74bbz CAR-T cell cytolysis (FIG. 5E). Among the lymphoid cells, most of the normal B cells (93.1%) were CD74+ with a median density of 7315 molecules per cell while only a subset of CD8+ cytotoxic T (T_{CYTO}) cells (16.7% CD74+, median density 2687 molecules per cell), and CD4+T helper (T_{H}) cells (10.4% CD74+, median density 56 molecules per cell) expressed CD74 (FIG. 5F). Of note, physiologic activation of T cells (CD3/CD28 soluble antibody and IL-2) and B cells (LPS, 10 ng/mL, and anti-IgM, 10 ug/mL) did not affect CD74 expression or CD74-specific lysis (FIG. 5G, FIG. 11,). To characterize the activity of 42105-74bbz CAR-T cells on normal immune cell in vivo, a CD34+ hematopoietic stem cell humanized mouse model was developed and utilized. (Verma, 2020) Human CD34+ stem cells were isolated from umbilical cord blood and engrafted in NSG mice. The mice were intraperitoneally injected with stem cell factor (SCF), granulocyte macrophage colony-stimulating factor (GM-CSF) and IL-3 to promote the myeloid differentiation as previously reported. (Verma, 2020) After 10 weeks of human CD45+ cell reconstitution as determined in peripheral blood, mice were randomized to receive either 5×10⁶ autologous 42105-74bbz CAR-T cells generated from the same cord blood donor or UTT cells intravenously. As shown in FIG. 6A, 74bbz CAR-T cells, which peaked at Day 18 post CAR-T injections, did not have a significant effect on human B cells, monocytes, granulocytic, monocytic myeloid suppressors and NK cells (FIG. 6B; FIG. 12). Collectively, these in vitro and in vivo results show that while CD74 is expressed at variable levels on resting and activated immune cell subsets, 42105-74bbz CAR-T cells are significantly cytotoxic against normal B cells only.

42105-74Bbz CAR-T Cells Show Strong Antitumor Activity in a PDX Generated from a MCL Patient that Progressed after Commercial CD19 CAR-T Therapy

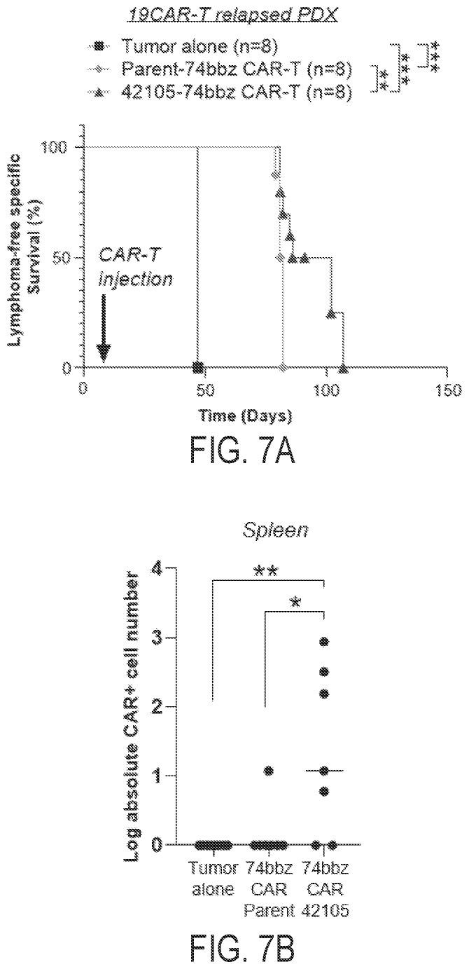

The activity of optimized 42105-74bbz CAR-T compared to the parent construct was determined in vivo. A newly established PDX generated from an MCL patient (patient #3, Table 3) was used that progressed after commercial CD19 CAR-T therapy. NSG mice were engrafted with 1×10⁶ of MCL cell from passage 4 and, in group of 8, randomized to receive control (tumor alone), 5×10⁶ 42105-74bbz CAR-T, or 5×10⁶ parent-74bbz CAR-T cells which were intravenously injected on Day 3. The engraftment of MCL cells was confirmed via weekly peripheral blood sampling and flow cytometry (human CD5+/CD19+ cells). All mice died of MCL with the presence of tumor cells in blood or spleen. Both parent and 42105-74bbz CAR-Ts treated animals survived significantly longer the control mice (median survival 47 days, p<0.0001, n=8). Mice treated with 42105-74bbz CAR-T cells survived significantly longer than those treated with parent-74bbz CAR-T cells (median survival 94 days vs 81.5, p value=0.005, n=8) (FIG. 7A). In support of the survival advantage, mice treated with 42105-74bbz CAR-Ts at early removal criteria (ERC) had a significantly higher number of CAR-Ts in the spleen compared to control as well as parent CARTs (FIG. 7B).

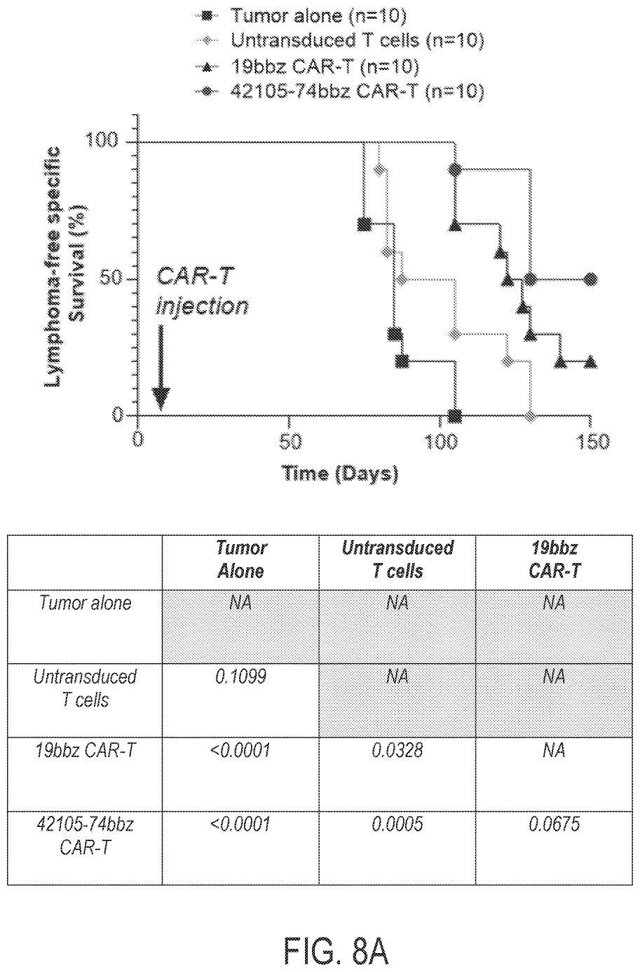

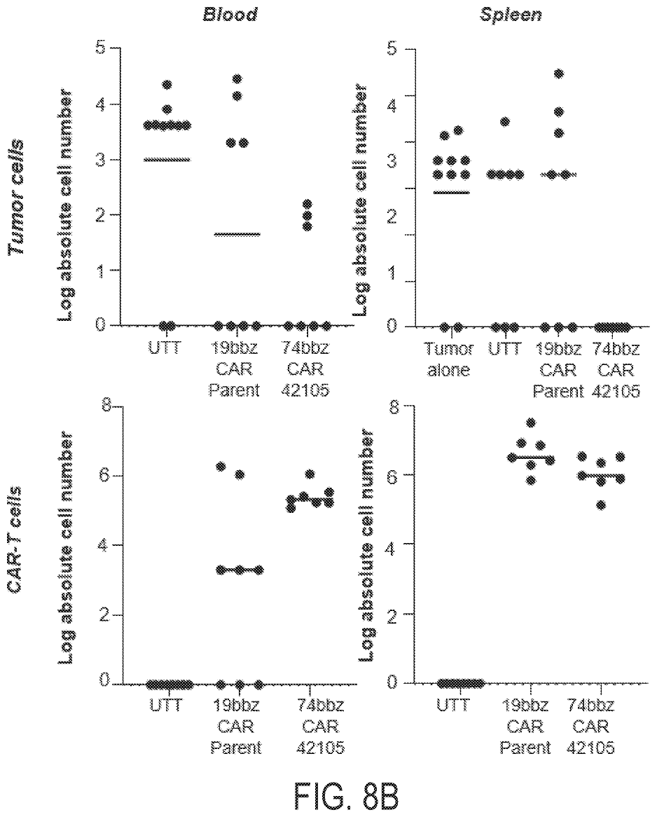

To determine if 42105-74bbz CAR-T treatment was non-inferior to the standard CD19 CAR-T therapy, a subcutaneous MCL xenograft model using NSG mice engrafted with Mino cells was adopted. The animals in groups of 10 were randomized to receive tumor alone, tumor+5×10⁶ UTT cells, tumor+5×10⁶ 19bbz CAR-T cells, and tumor+5×10⁶ 42105-74bbz CAR-T cells intratumorally 3 days after 1×10⁶ Mino cells were engrafted and when palpable tumors were detected. As shown in FIG. 8A, treatment with both 19bbz CAR-T cells and 42105-74bbz CAR-T cells significantly prolonged the mice survival compared to the tumor alone (p<0.0001, n=10) and UTT groups (p-value<0.0001 compared to untreated group, n=10). In addition, mice treated with 42105-74bbz CAR-T cells had a median survival of 56 days (n=10) compared to 50 days of those treated with 19bbz CAR-T cells, (n=10) however the survival advantage did not reach statistical significance (p-value=0.0675) despite the fact that mice treated with 42105-74bbz CAR-T cells had the smallest number of MCL cells in both peripheral blood and spleen (FIG. 8B). The circulating and tumor infiltrating CAR-T cells in the 42105-74bbz CAR-T cell treated mice remained high and comparable between the 2 CAR-T cell treated groups (FIG. 8B, p>0.05). Collectively, the data provide evidence that 42105-74bbz CAR-T is active in a PDX generated from a MCL patient that progressed after commercial CD19 CAR-T cells and is not inferior to 19bbz CAR-T cells.

### Discussion

Currently, the development of CAR-T therapy for MCL patients primarily focuses on CD19 as a target. In 2020, the US Food and Drug Administration (FDA) approved brexucabtagene autoleucel (Tecartus) which is a CD19 targeting CAR-T cell therapy for the treatment of MCL based on the results of the ZUMA-2 clinical trial, (Wang, 2016) offering a promising treatment option for this incurable disease. Discussed herein is a CAR-T cell therapy targeting CD74, an invariant chain of MHC class II HLA-DR which is overexpressed on MCL cells. 42105-74bbz CAR-T cells showed significant cytotoxicity against MCL cells in vitro and in vivo including a MCL PDX derived from a patient that relapsed after commercial CD19 CAR-T cell therapy. While CD74 is expressed at a lower level also on normal immune cell subsets, there was surprisingly minimal cytotoxicity of 42105-74bbz CAR-T cells in this context in vitro and in vivo.

The current treatment of MCL involves a multi-modal approach that includes chemotherapy, stem cell transplant, and targeted therapies. (Campo, 2015) NCCN guidelines recommend an intensive chemotherapy regimen followed by autologous stem cell transplantation and maintenance rituximab for patients who are physically fit and under 65. For the majority of MCL patients who are over 65, less aggressive treatment strategies are adopted. (Al-Mansour, 2022) Small molecule inhibitors of BTK including ibrutinib, acalabrutinib, zanubrutinib, and pirtobrutinib, and Bcl2 such as venetoclax are FDA approved for the treatment of relapsed/refractory MCL and provide significant clinical benefits for a period of time. Unfortunately, the vast majority of MCL patients treated with targeted therapies eventually progress and develop a more aggressive disease which results in low response rates to other treatment approaches and an overall short survival. Anti-CD19 KTE-X19 CAR-T cells (Tecartus) provides a promising cell therapy option for relapsed and refractory MCL. While the overall response rate and complete remission rates following treatment of MCL patients with Tecartus was high at 91% and 68%, respectively, the progression free survival and the overall survival at approximately 3 years of follow up were 25.8 and 46.6 months, respectively, suggesting that a significant portion of these patients will need additional treatment.

As reported by Wang et al. in their initial study, patients receiving KTE-X19 CAR-T cells experienced predictable CAR-T therapy adverse events such as cytopenias (grade 3 or higher in 94% of the patients), cytokine release syndrome (91% of patients, 15% of which were grade 3-4) and neurotoxicity (63% of patients, 31% of which were grade 3-4) which could be mediated by the expression of CD19 on brain pericytes. (Parker, 2020) This observation was supported by the analysis of multiple single-cell RNA sequencing datasets from adult samples from different brain regions and validated via immunohistochemistry with CD19 antibodies. (Parker, 2020) Interestingly, analysis of the same data set revealed that CD74 is the gene with highest differential expression between B cells and brain pericytes which were confirmed using flow cytometry. This shows that 74bbz CAR-T cell may not induce depletion of pericytes, endothelial activation, and cerebral edema.

While single agent milatuzumab has shown significant activity in preclinical models of B-cell malignancies, milatuzumab has not provided meaningful clinical benefits to patients with CLL, MM, and B-cell NHL, primary due to its very short half-life secondary to rapid internalization of the antibody-antigen complex and antigen sink. (Haran, 2018; Martin, 2015) While believing in CD74 as a novel therapeutic target and to overcome milatuzumab's limitations, a second-generation CAR construct was designed with 4-1BB and CD3ζ chain signaling domain by joining a scFV specifically targeting human CD74 with the CD8 hinge/transmembrane domain and CD3ζ and transduced the construct in CD74 negative central memory T cells minimizing the risk of CAR-Ts fratricide effect. In addition, the CAR-T cell construct contains a humanized scFv minimizing its immunogenicity and the risk for anti-CAR immunity. (Parker, 2020)

A strategy was adopted for the investigation of the 74bbz CAR-T cells, utilizing a variety of assays, including a flow cytometry-based antigen binding assay, T cell activation assay, repeated antigen stimulation assay, (Smith, 2018) and cytotoxicity assay. Through in-silico modeling of the binding of CD74 antigen to anti-CD74 scFV mutants, it was discovered that mutations on the V_{H} chain and CDR3 had the highest docking scores and produced mutants with higher T cell activation. However, increased binding affinity and T cell activation did not always lead to improved cytotoxicity and cell proliferation (FIG. 1).

The main limitation of targeting CD74 either with a naked monoclonal antibody or with a CAR-T cell product is its expression on normal immune cell subsets. (Barrera, 2005; Martin, 2015) To summarize, CD74 is expressed at variable levels on antigen presenting cells such as B cell, monocytes, activated T cells, in addition to a subset of myeloid cells. Interestingly, milatuzumab showed a favorable safety profile with the most common adverse event being infusion reactions. In the phase I-II clinical trial for patients with relapsed/refractory CLL, treatment with milatuzumab did not result in significant cytopenias. Interestingly, a significant increase in non-malignant lymphocyte counts following each infusion of milatuzumab was observed in those patients with a transient response to milatuzumab. (Haran, 2018) In the phase I dose escalation clinical trial with single agent milatuzumab in patients with relapsed/refractory MM, routine hematology laboratories showed no significant changes from baseline. (Kaufman, 2013) In the phase I study with milatuzumab monotherapy in patients with previously treated B-cell NHL the most common treatment-related grade 3-4 hematologic toxicities were neutropenia (9%), and thrombocytopenia (5%) but not lymphopenia. (Martin, 2015)

Overall CD74 expression is significantly lower in normal immune cell subsets compared to MCL cells. Among the normal immune cell subsets, myeloid and B cells expressed the highest level of CD74 compared with T_{H} and T_{CYTO} cells. Interestingly, the 42105-74bbz CAR-T cells failed to induce significant cytotoxicity in resting and activated normal T cells and CD33+ myeloid cells while the killing was significant against resting and activated B cells. Of note, there was no significant depletion of immune cell subsets in peripheral blood including B cell, monocytes, and NK cell in the humanized mouse model.

The differential cytotoxic effect of 42105-74bbz CAR-T cells on immune cells subsets could be partially explained by the pro-survival function played by CD74 in normal B cells. (Starlets, 2006) CD74 is indispensable for the proper B cell development and together with CD44 promotes B cell proliferation and survival by activation of downstream pathways such as NF-kB and PI3K/Akt signaling upon MIF binding. (Schröder, 2016) CD74 can also be expressed by epithelial cells of the gastrointestinal (GI) tract under inflammatory conditions (Barrera, 2005; Beswick, 2009) which is obviously a concern for potential toxicity following treatment with 42105-74bbz CAR-T cells. CD74 is post-translationally glycosylated and, in humans, it exists in 5 isoforms. (Schröder, 2016)

An interesting and relatively unexplored aspect of CD74 as a therapeutic target is that its surface expression can be pharmacologically modulated by inhibiting its degradation in the autophagosome/lysosomal compartment. (Alinari, FTY720 increases CD74 expression, 2011; Alinari, 2012) Enhancement of CD74 expression positively correlated with milatuzumab-mediated MCL cell death. (Alinari, FTY720 increases CD74 expression, 2011; Alinari, 2012). Data presented here indicated that the activity of CAR-T cells correlated positively with target antigen density providing the rationale for combination strategies to maximize the therapeutic potential of 42105-74bbz CAR-T cells. From a toxicity standpoint, it is important to note that the inhibition of CD74 degradation affect the expression of CD74 on lymphoma cells only leaving its expression on normal immune cells unaffected. This is likely because lymphoma cells rely on autophagy for survival significantly more so than normal immune cells. (Alinari, 2017)

### CONCLUSION

This is the first study describing and investigating the development of a CD74bbz CAR-T cell product. These findings provide evidence of significant activity of 42105-74bbz CAR-T cells in MCL models with surprisingly minimal toxicity in normal immune cell subsets.

Other advantages will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

TABLE 4
Protein Sequence SEQ ID NOS.

| SEQ ID | Sequence | |
|---|---|---|
| NO. | Name | Protein Sequence |
| 1 | Parent | DIQLTQSPLSLPVTLGQPASISCRSSQSLVHRNGNTYLHWFQQ |
| | | RPGQSPRLLIYTVSNRFSGVPDRFSGSGSGTDFTLKISRVEAE |
| | | DVGVYFCSQSSHVPPTFGAGTRLEIKGGGGSGGGGSGGGGS |
| | | QVQLQQSGSELKKPGASVKVSCKASGYTFTNYGVNWIKQAP |
| | | GQGLQWMGWINPNTGEPTFDDDFKGRFAFSLDTSVSTAYLQ |
| | | ISSLKADDTAVYFCSRSRGKNEAWFAYWGQGTLVTVSS |
| 2 | 42105-01 | DIQMTQTTSSLSASLGDRVTISCRASQSLVHRNGNTYLNWYQ |
| | | QKPDGTVKLLIYTVSRLHSGVPSRFSGSGSGTDYSLTISNLEQE |
| | | DIATYFCSQSSHVPPTFGGGTKLEITGGGGSGGGGSGGGGSEV |
| | | KLQESGPGLVAPSQSLSVTCTVSGYTFTNYGVSWIRQPPRKGL |
| | | EWLGVINPNTGEPYYNSALKSRLTIIKDNSKSQVFLKMNSLQT |
| | | DDTAIYYCARSRGKNEAWFAYWGQGTSVTVSS |
| 3 | 42105-02 | DIVLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYYCSQSSHVPPTFGQGTRLEIKGGGGSGGGGSGGGGSQV |
| | | QLVQSGSELKKPGASVKVSCKASGYTFTNYGVNWVRQAPGQ |
| | | GLEWMGWINPNTGEPTFDDDFKGRFVFSLDTSVSTAYLQISSL |
| | | KADDTAVYFCARSRGKNEAWFAYWGQGTLVTVSS |
| 4 | 42105-03 | DVVMTQSPLSLPVTLGQPASISCRSSQSLVHRNGNTYLHWFQ |
| | | QRPGQSPRRLIYTVSNRFSGVPDRFSGSGSGTDFTLKISRVEAE |
| | | DVGVYYCSQSSHVPPTFGQGTRLEIKQVQLVQSGSELKKPGA |
| | | SVKVSCKASGYTFTNYGVNWVRQAPGQGLEWMGWINPNTG |
| | | EPTFDDDFKGRFVFSLDTSVSTAYLQISSLKAEDTAVYYCARS |
| | | RGKNEAWFAYWGQGTLVTVSS |
| 5 | 42105-04 | DIQMTQTTSSLSASLGDRVTISCRASQSLVHRNGNTYLNWYQ |
| | | QKPDGTVKLGIYTVSRGHSGVPSRFSGSGSGTDYSLTISNLEQ |
| | | EDIATYFCSQSSHVPPTFGGGTKLEITGGGGSGGGGSGGGGSE |
| | | VKLQESGPGLVAPSQSLSVTCTVSGYTFTNYGVSWIRQPPRK |
| | | GLEWLGVINPNTGEPYYNSALKSRLTIIKDNSKSQVFLKMNSL |
| | | QTDDTAIYYCARSRGKNEAWFAYWGQGTSVTVSS |
| 6 | 42105-05 | DIVDTQSPLSLPVTLGQPASISCRSSQSLVHRNGNTYLHWFQQ |
| | | RPGQSPRLGIPTVSNRFSGVPDRFSGSGSGTDFTLKISRVEAED |
| | | VGVYYCSQSSHVPPTFGQGTRLEIKQVQLVQSGSELKKPGAS |
| | | VKVSCKASGYTFTNYGVNWVRQAPGQGLEWMGWINPNTGE |
| | | PTFDDDFKGRFVFSLDTSVSTAGLQISSLKADDTAVYFCARSR |
| | | GKNEAWFAYWGQGTLVTVSS |
| 7 | 42105-06 | DVVMTQSPLSLPVTLGQPASISCRSSQSLVHRNGNTYLHWFQ |
| | | QRPGQSPRRLPYTVSNRFSGVPDRFSGSGSGTDFTLKISRVEA |
| | | EDVGVYYCSQSSHVPPTFGQGTRLEIKQVQLVQSGSELKKPG |
| | | ASVKVSCKASGYTFTNYGVNWVRQAPGQGLEWMGWINPNT |
| | | GEPTFDDDFKGRFVFSLDTSVSTAGLQISSLKAEDTAVYYCAR |
| | | SRGKNEAWFAYWGQGTLVTVSS |
| 8 | 5311-01 | DIQMTQTTSSLSASLGDRVTISCRASQSLVHRNGNTYLNWYQ |
| | | QKPDGTVKLLIYTVSRLHSGVPSRFSGSGSGTDYSLTISNLEQE |
| | | DIATYFCSQSSHVPPTFGGGTKLEITGGGGSGGGGSGGGGSQV |
| | | QLQQSGSELKKPGASVKVSCKASGYTFTNYGVNWIKQAPGQ |
| | | GLQWMGWINPNTGEPTYDDDFKGRFAFSLDTSVSTAYLQISS |
| | | LKADDTAVYFCSRSRGKNEAWFAYWGQGTLVTVSS |
| 9 | 5311-02 | DIVLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYYCSQSSHVPPTFGQGTRLEIKGGGGSGGGGSGGGGSQV |
| | | QLVQSGSELKKPGASVKVSCKASGYTFTNYGVNWVRQAPGQ |
| | | GLEWMGWINPNTGEPTYDDDFKGRFVFSLDTSVSTAYLQISS |
| | | LKADDTAVYFCARSRGKNEAWFAYWGQGTLVTVSS |
| 10 | 5311-03 | DVVMTQSPLSLPVTLGQPASISCRSSQSLVHRNGNTYLHWFQ |
| | | QRPGQSPRRLIYTVSNRFSGVPDRFSGSGSGTDFTLKISRVEAE |
| | | DVGVYYCSQSSHVPPTFGQGTRLEIKGGGGSGGGGSGGGGSQ |
| | | VQLVQSGSELKKPGASVKVSCKASGYTFTNYGVNWVRQAPG |
| | | QGLEWMGWINPNTGEPTYDDDFKGRFVFSLDTSVSTAYLQIS |
| | | SLKAEDTAVYYCARSRGKNEAWFAYWGQGTLVTVSS |
| 11 | 5311-04 | DIVDTQSPLSLPVTLGQPASISCRSSQSLVHRNGNTYLHWFQQ |
| | | RPGQSPRLGIPTVSNRFSGVPDRFSGSGSGTDFTLKISRVEAED |
| | | VGVYYCSQSSHVPPTFGQGTRLEIKGGGGSGGGGSGGGGSQV |
| | | QLVQSGSELKKPGASVKVSCKASGYTFTNYGVNWVRQAPGQ |
| | | GLEWMGWINPNTGEPTYDDDFKGRFVFSLDTSVSTAGLQISS |
| | | LKADDTAVYFCARSRGKNEAWFAYWGQGTLVTVSS |
| 12 | 5311-05 | DVVMTQSPLSLPVTLGQPASISCRSSQSLVHRNGNTYLHWFQ |
| | | QRPGQSPRRLPYTVSNRFSGVPDRFSGSGSGTDFTLKISRVEA |
| | | EDVGVYYCSQSSHVPPTFGQGTRLEIKGGGGSGGGGSGGGGS |
| | | QVQLVQSGSELKKPGASVKVSCKASGYTFTNYGVNWVRQAP |
| | | GQGLEWMGWINPNTGEPTYDDDFKGRFVFSLDTSVSTAGLQI |
| | | SSLKAEDTAVYYCARSRGKNEAWFAYWGQGTLVTVSS |
| 13 | 543 | DIQMTQSPSSLSASVGDRVTITCRASQGISSWLAWYQQKPEK |
| | | APKSLIYAASSLQSGVPSRFSGSGSGTDFTLTISSLQPEDFATY |
| | | YCQQYNSYPLTFGGGTKVEIKGGGGSGGGGSGGGGSQVQLV |
| | | ESGGGVVQPGRSLRLSCAASGFTFSSYGMHWVRQAPDKGLE |
| | | WVAVIWYDGSNKYYADSVKGRFTISRDNSKNTLYLQMNSLR |
| | | AEDTAVYYCARGGTLVRGAMYGTDVWGQGTTVTVSS |
| 14 | 543-W | DIQMTQSPSSLSASVGDRVTITCRASQGISSWLAWYQQKPGK |
| | | APKLLIYAASSLQSGVPSRFSGSGSGTDFTLTISSLQPEDFATY |
| | | YCQQYNSYPLTFGGGTKVEIKGGGGSGGGGSGGGGSQVQLV |
| | | ESGGGVVQPGRSLRLSCAASGFTFSSYGMHWVRQAPGKGLE |
| | | WVAVIWYDGSNKYYADSVKGRFTISRDNSKNTLYLQMNSLR |
| | | AEDTAVYYCARGGTLVRGAMYGTDVWGQGTTVTVSS |
| 15 | 553 | DIQLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYFCSQSSHVPPTFGAGTRLEIKGGGGSGGGGSGGGGSQV |
| | | QLQESGGGSVQAGGSLRLSCVASGLVYSLKAYDWGWARQA |
| | | PGKECELVGRIMPNGATVYIDSVKGRFIISRENSNMTVYLQM |
| | | NSLKFEDTAVYYCARVGDPSGGWCKGQGTQVTVSS |
| 16 | 553-W | DIVDTQSPLSLPVTLGQPASISCRSSQSLVHRNGNTYLHWFQQ |
| | | RPGQSPRLGIPTVSNRFSGVPDRFSGSGSGTDFTLKISRVEAED |
| | | VGVYYCSQSSHVPPTFGQGTRLEIKGGGGSGGGGSGGGGSQV |
| | | QLVESGGGLVQPGGSLRLSCVASGLTFSLKAYDWGWIRQAP |
| | | GKALEWVGRIMPNGATVYIDSVKGRFTISRDNSKNTVYLQM |
| | | NSLKAEDTAVYYCARVGDPSGGWCWGQGTLVTVSS |
| 17 | 563 | DIQMTQSPSSVSASVGDRVTITCRASQGISSWVAWYQQKPGK |
| | | APKLLIYGSSRLQSGVPSRFSGSGSGTDFTLTISSLQPEDFATY |
| | | YCQQYNTYPLTFGGGTKVEIKGGGGSGGGGSGGGGSQVQLV |
| | | ESGGGVVQPGRSLRLSCAASGFNFSDYGMHWVRQAPDKGLE |
| | | WVAVIYDGSISYYADSVKGRFTISRDNSKNTLYLQMNSLRAE |
| | | DTAVYYCARGGTVEHGAVYGTDVWGQGATVTVSS |
| 18 | 563-W | DIQMTQSPSSVSASVGDRVTITCRASQGISSWVAWYQQKPGK |
| | | APKLLIYGSSRLQSGVPSRFSGSGSGTDFTLTISSLQPEDFATY |
| | | YCQQYNTYPLTFGGGTKVEIKGGGGSGGGGSGGGGSQVQLV |
| | | ESGGGVVQPGRSLRLSCAASGFTFSDYGMHWVRQAPGKGLE |
| | | WVAVIDDGSISYYADSVKGRFTISRDNSKNTLPLQMNSLRAE |
| | | DTAVYYCARGGTVEHGAVYGTDVWGQGTTVTVSS |
| 19 | 5321 | DIQMTQTTSSLSASLGDRVTISCRASQSLVHRNGNTYLNWYQ |
| | | QKPDGTVKLLIYTVSRLHSGVPSRFSGSGSGTDYSLTISNLEQE |
| | | DIATYFCSQSSHVPPTFGGGTKLEITGGGGSGGGGSGGGGSEV |
| | | KLQESGPGLVAPSQSLSVTCTVSGYTFTNYGVSWIRQPPRKGL |
| | | EWLGVINPNTGEPYYNSALKSRLTIIKDNSKSQVFLKMNSLQT |
| | | DDTAIYYCSRCRGKNEAWFAYWGQGTSVTVSS |
| 20 | 534 | DIQMTQTTSSLSASLGDRVTISCRASQSLVHRNGNTYLNWYQ |
| | | QKPDGTVKLLIYTVSRLHSGVPSRFSGSGSGTDYSLTISNLEQE |
| | | DIATYFCSQSSHVPPTFGGGTKLEITGGGGSGGGGSGGGGSQV |
| | | QLQQSGSELKKPGASVKVSCKASGYTFTNYNVNWIKQAPGQ |
| | | GLQWMGWINPNTGEPTADDDFKGRFAFSLDTSVSTAYLQISS |
| | | LKADDTAVYFCSRSRGKNEAWFAYWGQGTLVTVSS |
| 21 | 532 | DIQMTQTTSSLSASLGDRVTISCRASQSLVHRNGNTYLNWYQ |
| | | QKPDGTVKLLIYTVSRLHSGVPSRFSGSGSGTDYSLTISNLEQE |
| | | DIATYFCSQSSHVPPTFGGGTKLEITGGGGGGGGSGGGGSQV |
| | | QLQQSGSELKKPGASVKVSCKASGYTFTNYGMNWIKQAPGQ |
| | | GLQWMGWINPNTGEPTADDDFKGRFAFSLDTSVSTAYLQISS |
| | | LKADDTAVYFCSRSRGKNEAWFAYWGQGTLVTVSS |
| 22 | 5310 | DIQMTQTTSSLSASLGDRVTISCRASQSLVHRNGNTYLNWYQ |
| | | QKPDGTVKLLIYTVSRLHSGVPSRFSGSGSGTDYSLTISNLEQE |
| | | DIATYFCSQSSHVPPTFGGGTKLEITGGGGSGGGGSGGGGSQV |
| | | QLQQSGSELKKPGASVKVSCKASGYTFTNYGVNWIKQAPGQ |
| | | GLQWMGWINPNTGEPTFDDDFWGRFAFSLDTSVSTAYLQISS |
| | | LKADDTAVYFCSRSRGKNEAWFAYWGQGTLVTVSS |
| 23 | 5319 | DIQMTQTTSSLSASLGDRVTISCRASQSLVHRNGNTYLNWYQ |
| | | QKPDGTVKLLIYTVSRLHSGVPSRFSGSGSGTDYSLTISNLEQE |
| | | DIATYFCSQSSHVPPTFGGGTKLEITGGGGSGGGGSGGGGSQV |
| | | QLQQSGSELKKPGASVKVSCKASGYTFTNYGVNWIKQAPGQ |
| | | GLQWMGWINPNTGEPTFDDDFWGRFAFSLDTSVSTAYLQISS |
| | | LKADDTAVYFCSRSREKNEAWFAYWGQGTLVTVSS |
| 24 | 5315 | DIQMTQTTSSLSASLGDRVTISCRASQSLVHRNGNTYLNWYQ |
| | | QKPDGTVKLLIYTVSRLHSGVPSRFSGSGSGTDYSLTISNLEQE |
| | | DIATYFCSQSSHVPPTFGGGTKLEITGGGGSGGGGSGGGGSQV |
| | | QLQQSGSELKKPGASVKVSCKASGYTFTNYGVNWIKQAPGQ |
| | | GLQWMGWINPNTGEPTFDDDFWGRFAFSLDTSVSTAYLQISS |
| | | LKADDTAVYFCCRcREKNEAWFAYWGQGTLVTVSS |
| 25 | 571 | DIQLTQSPLSLPVTLGQPASISCRSSQSEVHRNGNTYLHWFQQ |
| | | RPGQSPRLLIYTVSNRFSGVPDRFSGSGSGTDFTLKISRVEAED |
| | | VGVYFCSQSSHVPPTFGAGTRLEIKGGGGSGGGGSGGGGSQV |
| | | QLQQSGSELKKPGASVKVSCKASGYTFTNYGVNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 26 | 573 | DIQLTQSPLSLPVTLGQPASISCRSSQSLWHRNGNTYLHWFQQ |
| | | RPGQSPRLLIYTVSNRFSGVPDRFSGSGSGTDFTLKISRVEAED |
| | | VGVYFCSQSSHVPPTFGAGTRLEIKGGGGSGGGGSGGGGSQV |
| | | QLQQSGSELKKPGASVKVSCKASGYTFTNYGVNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 27 | 576 | DIQLTQSPLSLPVTLGQPASISCRSSQSLVHRNGNTYCHWFQQ |
| | | RPGQSPRLLIYTVSNRFSGVPDRFSGSGSGTDFTLKISRVEAED |
| | | VGVYFCSQSSHVPPTFGAGTRLEIKGGGGSGGGGSGGGGSQV |
| | | QLQQSGSELKKPGASVKVSCKASGYTFTNYGVNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 28 | 578 | DIQLTQSPLSLPVTLGQPASISCRSSQSLVHRNGNTYLHWFQQ |
| | | RPGQSPRLLIYEVSNRFSGVPDRFSGSGSGTDFTLKISRVEAED |
| | | VGVYFCSQSSHVPPTFGAGTRLEIKGGGGSGGGGSGGGGSQV |
| | | QLQQSGSELKKPGASVKVSCKASGYTFTNYGVNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 29 | 579 | DIQLTQSPLSLPVTLGQPASISCRSSQSLVHRNGNTYLHWFQQ |
| | | RPGQSPRLLIYTESNRFSGVPDRFSGSGSGTDFTLKISRVEAED |
| | | VGVYFCSQSSHVPPTFGAGTRLEIKGGGGSGGGGSGGGGSQV |
| | | QLQQSGSELKKPGASVKVSCKASGYTFTNYGVNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 30 | 5711 | DIQLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYFCSCSSHVPPTFGAGTRLEIKGGGGSGGGGSGGGGSQV |
| | | QLQQSGSELKKPGASVKVSCKASGYTFTNYGVNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 31 | 5718 | DIQLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYFCSQSSHWPPTFGAGTRLEIKGGGGSGGGGSGGGGSQ |
| | | VQLQQSGSELKKPGASVKVSCKASGYTFTNYGVNWIKQAPG |
| | | QGLQWMGWINPNTGEPTFDDDFKGRFAFSLDTSVSTAYLQIS |
| | | SLKADDTAVYFCSRSRGKNEAWFAYWGQGTLVTVSS |
| 32 | 5721 | DIQLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYFCSQSSHVPPCFGAGTRLEIKGGGGSGGGGSGGGGSQV |
| | | QLQQSGSELKKPGASVKVSCKASGYTFTNYGVNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 33 | 532 | DIQLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYFCSQSSHVPPTFGAGTRLEIKGGGGSGGGGSGGGGSQV |
| | | QLQQSGSELKKPGASVKVSCKASGYTFTNYGMNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 34 | 536 | DIQLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYFCSQSSHVPPTFGAGTRLEIKGGGGSGGGGSGGGGSQV |
| | | QLQQSGSELKKPGASVKVSCKASGYTFTNYWVNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 35 | 537 | DIQLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYFCSQSSHVPPTFGAGTRLEIKGGGGSGGGGSGGGGSQV |
| | | QLQQSGSELKKPGASVKVSCKASGYTFTNYGVNWIKQAPGQ |
| | | GLQWMGWINPNTGEPTFDDDFKGRFAFSLDTSVSTAYLQISS |
| | | LKADDTAVYFCSRSRGKNEAWFAYWGQGTLVTVSS |
| 36 | 5313 | DIQLTQSPLSLPVTLGQPASISCRSSQSLVHRNGNTYLHWFQQ |
| | | RPGQSPRLLIYTVSNRFSGVPDRFSGSGSGTDFTLKISRVEAED |
| | | VGVYFCSQSSHVPPTFGAGTRLEIKGGGGSGGGGSGGGGSQV |
| | | QLQQSGSELKKPGASVKVSCKASGYTFTNYGVNWIKQAPGQ |
| | | GLQWMGWINPNTGEPTWDDDFKGRFAFSLDTSVSTAYLQISS |
| | | LKADDTAVYFCSRSRGKNEAWFAYWGQGTLVTVSS |
| 37 | CAR | TTTPAPRPPTPAPTIASQPLSLRPEACRPAAGGAVHTRGLDFA |
| | backbone | CDIYIWAPLAGTCGVLLLSLVITLYCKRGRKKLLYIFKQPFMR |
| | | PVQTTQEEDGCSCRFPEEEEGGCELRVKFSRSADAPAYQQGQ |
| | | NQLYNELNLGRREEYDVLDKRRGRDPEMGGKPRRKNPQEGL |
| | | YNELQKDKMAEAYSEIGMKGERRRGKGHDGLYQGLSTATK |
| | | DTYDALHMQALPPR* |

TABLE 5
DNA Sequence SEQ ID NOS.

| SEQ ID | Sequence | |
|---|---|---|
| NO. | Name | DNA Sequence |
| 38 | Parent | GATATTCAGTTGACTCAATCTCCCCTCTCTCTCCCCGTTAC |
| | | CCTGGGCCAACCTGCCTCCATATCATGTCGGTCCTCACAA |
| | | TCACTTGTACACCGCAATGGGAACACTTACCTTCACTGGT |
| | | TCCAGCAACGGCCAGGTCAATCTCCACGGTTGCTGATATA |
| | | TACTGTAAGCAATCGGTTTAGTGGTGTCCCTGACCGCTTC |
| | | AGTGGTTCAGGGTCAGGAACTGACTTTACCCTGAAAATTA |
| | | GTCGGGTGGAGGCTGAGGATGTAGGCGTCTACTTTTGCAG |
| | | CCAGTCCTCACACGTTCCGCCAACGTTTGGTGCGGGTACT |
| | | AGACTTGAAATAAAGGGTGGCGGCGGAAGTGGCGGCGGG |
| | | GGTTCTGGTGGAGGCGGTTCCCAGGTGCAACTTCAACAAA |
| | | GCGGTAGTGAACTGAAGAAGCCGGGGGCGAGCGTCAAGG |
| | | TATCCTGTAAGGCTTCAGGCTATACGTTCACCAACTATGG |
| | | CGTCAACTGGATCAAGCAGGCACCGGGCCAGGGATTGCA |
| | | ATGGATGGGCTGGATAAATCCAAACACGGGAGAGCCTAC |
| | | CTTTGATGACGATTTTAAAGGACGGTTTGCATTTAGTCTTG |
| | | ACACGAGCGTGTCAACGGCGTACTTGCAGATATCATCTCT |
| | | CAAGGCCGACGATACGGCAGTGTATTTTTGTTCTCGGTCA |
| | | AGGGGCAAAAACGAGGCATGGTTTGCGTATTGGGGTCAG |
| | | GGGACGCTGGTCACAGTGTCCTCT |
| 39 | 42105-01 | GATATACAAATGACCCAGACCACTTCAAGTTTGTCAGCCA |
| | | GCTTGGGGGACAGGGTCACTATATCATGCCGAGCGTCTCA |
| | | GTCTTTGGTCCATCGCAATGGGAATACCTATCTTAACTGGT |
| | | ATCAACAGAAACCCGATGGCACGGTCAAACTGTTGATATA |
| | | CACCGTTTCCCGACTTCATAGCGGGGTGCCTAGTCGATTTT |
| | | CCGGATCCGGCTCCGGTACGGATTATTCTTTGACGATCTCC |
| | | AACCTGGAACAGGAGGATATTGCAACGTATTTTTGCTCAC |
| | | AGAGTTCACATGTACCCCCGACGTTCGGTGGTGGTACGAA |
| | | ACTCGAGATCACTGGCGGGGGCGGTTCAGGAGGCGGTGGA |
| | | TCTGGTGGAGGAGGATCCGAGGTAAAACTGCAAGAATCTG |
| | | GACCAGGTCTGGTGGCTCCTTCACAGAGCCTTTCCGTCACA |
| | | TGCACAGTCTCTGGTTACACGTTCACCAATTATGGAGTTTC |
| | | ATGGATTAGGCAACCGCCAAGAAAAGGTCTCGAGTGGCTC |
| | | GGGGTGATTAATCCCAACACGGGTGAGCCTTATTATAATTC |
| | | AGCCTTGAAGTCTCGCCTTACGATTATAAAAGACAACTCA |
| | | AAGTCACAAGTATTCCTGAAAATGAACTCCCTGCAGACCG |
| | | ATGATACGGCGATTTATTACTGCGCCAGATCACGAGGAAA |
| | | AAATGAAGCTTGGTTCGCGTACTGGGGCCAAGGGACAAGT |
| | | GTGACAGTAAGCAGT |
| 40 | 42105-02 | GACATCGTCTTGACACAATCCCCCCTTTCCCTTCCTGTCAC |
| | | GCTTGGCCAGCCCGCCTCTATCAGTTGTAGATCCAGCCAAA |
| | | GTCTGGTACACCGGAATGGGAATACATATCTGCATTGGTTC |
| | | CAGCAACGGCCAGGTCAGAGTCCTAGGTTGCTTATTTATAC |
| | | GGTCTCCAACCGGTTCAGTGGAGTACCCGACCGGTTTTCTG |
| | | GCTCAGGCAGCGGAACCGACTTTACATTGAAAATTTCACG |
| | | CGTCGAAGCCGAGGATGTTGGTGTGTATTATTGTTCCCAAA |
| | | GCTCTCACGTCCCCCCGACTTTTGGTCAGGGAACTAGGCTT |
| | | GAAATCAAAGGTGGCGGGGGAAGTGGTGGCGGGGGATCT |
| | | GGGGGTGGGGGATCTCAAGTGCAGCTCGTGCAGTCCGGGA |
| | | GCGAATTGAAAAAACCAGGGGCAAGCGTGAAGGTTTCTTG |
| | | TAAAGCCTCTGGATATACTTTCACCAATTACGGGGTAAATT |
| | | GGGTTCGGCAAGCACCAGGACAAGGACTGGAGTGGATGG |
| | | GTTGGATTAATCCAAATACGGGCGAGCCCACGTTTGACGA |
| | | TGACTTTAAGGGGCGATTTGTATTCAGCTTGGACACAAGCG |
| | | TGTCCACAGCGTACCTTCAAATATCATCTCTTAAGGCGGAC |
| | | GATACTGCAGTCTACTTCTGTGCTCGGAGTCGGGGAAAAA |
| | | ACGAGGCATGGTTTGCTTATTGGGGTCAGGGCACGCTTGTT |
| | | ACTGTCTCTTCT |
| 41 | 42105-03 | GATGTTGTTATGACACAGTCCCCATTGAGCCTCCCAGTGAC |
| | | TTTGGGACAGCCTGCGAGTATATCCTGTAGGAGCTCCCAAT |
| | | CACTCGTTCACCGAAATGGTAACACGTACCTGCACTGGTTT |
| | | CAGCAAAGGCCAGGTCAATCACCTCGAAGACTCATCTACA |
| | | CGGTCAGCAACCGCTTCTCTGGGGTGCCGGACAGGTTTTCC |
| | | GGGTCAGGCTCCGGTACAGACTTTACACTGAAAATCTCCA |
| | | GAGTGGAGGCAGAAGATGTAGGCGTTTATTACTGCTCCCA |
| | | AAGTAGTCATGTCCCTCCTACTTTCGGGCAAGGGACTAGAC |
| | | TTGAGATAAAACAAGTCCAACTCGTTCAATCCGGATCAGA |
| | | ACTGAAAAAGCCGGGTGCATCCGTCAAAGTCAGTTGCAAA |
| | | GCGAGTGGATACACGTTCACTAATTATGGTGTTAATTGGGT |
| | | TCGGCAGGCGCCAGGTCAAGGGTTGGAGTGGATGGGTTGG |
| | | ATTAACCCTAATACCGGCGAACCGACATTCGATGACGACT |
| | | TCAAAGGCCGATTCGTCTTCAGTCTTGACACTTCAGTAAGT |
| | | ACCGCTTATCTTCAGATCAGCAGTTTGAAAGCTGAAGACA |
| | | CCGCGGTGTACTACTGCGCACGGAGTAGGGGAAAGAATGA |
| | | AGCCTGGTTCGCATATTGGGGACAGGGGACGCTGGTGACC |
| | | GTCAGCAGC |
| 42 | 42105-04 | GACATACAGATGACTCAAACGACGAGCTCACTGTCCGCGT |
| | | CACTTGGCGACAGAGTGACGATCAGCTGCCGCGCGAGCCA |
| | | ATCTTTGGTACACCGGAACGGTAACACCTATCTTAACTGGT |
| | | ACCAACAAAAGCCCGATGGGACAGTCAAACTGGGAATCTA |
| | | CACTGTCTCTCGGGGCCATAGCGGAGTCCCCTCTCGCTTTA |
| | | GCGGATCAGGATCTGGTACTGACTATTCCCTTACTATCTCT |
| | | AACCTGGAACAAGAAGACATAGCCACCTATTTCTGTTCAC |
| | | AAAGCTCTCATGTACCTCCGACTTTCGGGGGAGGGACTAA |
| | | ACTTGAGATCACTGGTGGTGGCGGGAGCGGAGGAGGGGG |
| | | AAGTGGTGGCGGTGGATCAGAGGTGAAGCTTCAAGAATCC |
| | | GGACCGGGCCTCGTCGCCCCTTCACAAAGCCTGAGTGTGA |
| | | CTTGTACGGTGAGCGGATACACGTTTACTAACTATGGCGTA |
| | | TCTTGGATAAGACAGCCGCCTCGCAAGGGATTGGAGTGGC |
| | | TCGGGGTAATAAACCCCAACACCGGAGAGCCCTATTACAA |
| | | TTCTGCTCTCAAGTCAAGACTCACGATTATCAAAGATAACT |
| | | CAAAGTCTCAGGTTTTTCTGAAAATGAACAGTCTTCAAACG |
| | | GATGACACCGCAATCTACTACTGTGCTCGATCCCGAGGCA |
| | | AAAATGAGGCATGGTTTGCGTACTGGGGTCAAGGGACCTC |
| | | CGTAACAGTCTCTTCC |
| 43 | 42105-05 | GACATTGTAGATACGCAATCTCCCCTTTCCTTGCCCGTGAC |
| | | GTTGGGCCAGCCGGCCTCCATAAGCTGCCGATCAAGTCAG |
| | | TCTCTCGTCCACAGGAATGGGAACACTTACCTCCATTGGTT |
| | | CCAGCAACGACCCGGGCAATCACCGCGACTGGGCATACCC |
| | | ACTGTAAGCAATAGATTTAGCGGCGTACCTGACAGATTCTC |
| | | TGGTAGTGGCAGTGGCACAGACTTTACCCTCAAAATCAGT |
| | | CGCGTTGAAGCTGAGGACGTTGGCGTGTACTACTGCTCAC |
| | | AAAGCTCCCACGTACCGCCCACATTTGGTCAAGGAACGCG |
| | | CCTTGAAATAAAACAAGTACAGCTCGTCCAGAGCGGCTCA |
| | | GAACTCAAAAAACCGGGGGCTTCAGTGAAAGTATCTTGCA |
| | | AAGCAAGTGGGTATACGTTCACGAATTACGGCGTTAACTG |
| | | GGTAAGGCAAGCGCCTGGGCAGGGCCTTGAGTGGATGGGT |
| | | TGGATAAATCCTAATACAGGTGAGCCAACTTTTGATGACG |
| | | ACTTTAAGGGACGGTTTGTTTTCTCACTCGACACTTCTGTA |
| | | AGTACGGCAGGGCTCCAAATCTCTAGTCTTAAAGCTGACG |
| | | ACACAGCTGTTTATTTCTGCGCTAGAAGTCGCGGGAAGAA |
| | | CGAGGCGTGGTTTGCGTATTGGGGCCAGGGTACGCTCGTA |
| | | ACCGTTAGCTCT |
| 44 | 42105-06 | GACGTTGTTATGACTCAAAGCCCTCTGTCACTTCCTGTCAC |
| | | TCTTGGGCAACCCGCCAGTATCTCATGTCGAAGCTCTCAGA |
| | | GTCTCGTACACCGGAATGGGAATACATATCTTCACTGGTTC |
| | | CAACAGCGCCCAGGACAATCTCCCCGACGCCTTCCATATA |
| | | CCGTCAGCAATAGATTTTCAGGAGTACCTGACCGGTTCTCA |
| | | GGTTCTGGTTCAGGTACCGATTTTACACTTAAGATAAGCCG |
| | | GGTAGAAGCCGAAGATGTGGGGGTCTACTATTGTAGCCAG |
| | | AGTTCACACGTTCCCCCGACGTTTGGGCAAGGCACGCGCCT |
| | | CGAGATCAAGCAGGTCCAACTGGTCCAGAGCGGGTCAGAG |
| | | CTGAAGAAGCCCGGTGCGTCAGTTAAGGTCTCCTGCAAAG |
| | | CCTCAGGATATACCTTTACCAATTACGGAGTGAACTGGGTG |
| | | AGACAAGCTCCGGGTCAGGGGTTGGAGTGGATGGGATGGA |
| | | TAAATCCCAATACGGGCGAACCAACATTCGATGACGACTT |
| | | TAAAGGCAGATTCGTATTCTCATTGGACACGTCTGTCAGTA |
| | | CAGCAGGACTGCAAATATCATCCCTGAAAGCAGAGGACAC |
| | | GGCAGTTTACTATTGTGCGCGAAGCCGGGGAAAGAATGAA |
| | | GCTTGGTTTGCTTACTGGGGACAGGGCACCCTGGTAACCGT |
| | | CTCCTCT |
| 45 | 5311-01 | GACATCCAAATGACTCAGACTACCTCCTCACTCTCAGCGAG |
| | | CCTCGGCGATCGAGTTACGATCTCTTGTAGAGCAAGCCAA |
| | | AGTTTGGTTCATAGAAACGGAAACACATATCTGAATTGGT |
| | | ATCAGCAAAAACCGGACGGGACTGTGAAACTTCTGATCTA |
| | | CACAGTGTCTCGCTTGCATAGTGGGGTCCCAAGCAGGTTCT |
| | | CTGGTAGTGGCTCAGGTACTGACTATTCACTCACCATCTCC |
| | | AACCTTGAACAAGAAGATATCGCCACCTATTTCTGCTCCCA |
| | | GAGCTCACACGTCCCACCCACCTTTGGCGGGGGCACCAAG |
| | | CTGGAGATTACCGGGGGAGGCGGTAGTGGGGGTGGGGGA |
| | | AGTGGAGGTGGTGGCTCCCAAGTCCAGCTGCAACAATCTG |
| | | GTAGCGAGTTGAAGAAACCTGGGGCTAGTGTTAAAGTTAG |
| | | TTGCAAGGCCTCCGGATATACCTTCACGAATTACGGGGTA |
| | | AACTGGATCAAGCAAGCTCCCGGACAAGGACTGCAGTGGA |
| | | TGGGTTGGATTAACCCCAACACAGGAGAACCGACGTACGA |
| | | TGACGATTTCAAAGGGCGGTTCGCCTTCTCCTTGGATACAT |
| | | CTGTTTCCACCGCCTATCTCCAAATTTCTTCTCTCAAAGCCG |
| | | ACGACACAGCTGTGTATTTTTGTTCACGCTCACGCGGTAAG |
| | | AACGAAGCTTGGTTCGCGTACTGGGGCCAAGGGACCCTTG |
| | | TAACGGTGTCAAGC |
| 46 | 5311-02 | GATATCGTACTGACGCAATCTCCGCTGAGCCTGCCAGTTAC |
| | | TTTGGGCCAGCCTGCAAGCATCTCTTGTAGGTCATCACAAA |
| | | GTCTTGTCCACCGAAATGGCAACACATATCTGCATTGGTTT |
| | | CAGCAAAGGCCGGGTCAGAGTCCCCGCTTGCTGATATACA |
| | | CTGTGTCCAATCGGTTCTCAGGGGTGCCAGACAGGTTTTCT |
| | | GGAAGCGGAAGTGGGACTGACTTTACGCTTAAAATATCCC |
| | | GAGTAGAAGCGGAAGATGTCGGAGTGTACTACTGTTCCCA |
| | | GAGCTCCCATGTGCCTCCGACTTTCGGTCAGGGGACAAGG |
| | | CTTGAAATCAAGGGCGGGGGCGGTTCAGGCGGTGGAGGAT |
| | | CTGGCGGAGGTGGATCCCAGGTCCAGTTGGTGCAAAGCGG |
| | | TTCTGAACTGAAAAAGCCTGGAGCATCTGTAAAAGTGAGC |
| | | TGCAAAGCTAGTGGCTATACGTTTACTAACTATGGTGTCAA |
| | | CTGGGTTAGGCAGGCGCCGGGGCAGGGATTGGAATGGATG |
| | | GGCTGGATTAATCCTAACACCGGTGAACCGACTTACGATG |
| | | ACGACTTCAAAGGCCGCTTTGTGTTCAGCCTCGATACGAGC |
| | | GTCAGTACCGCATACTTGCAAATATCAAGTCTCAAAGCAG |
| | | ACGACACAGCAGTTTATTTCTGTGCTCGGTCACGGGGTAAA |
| | | AATGAAGCGTGGTTTGCATACTGGGGCCAAGGAACATTGG |
| | | TAACCGTTAGCAGC |
| 47 | 5311-03 | GACGTGGTAATGACTCAGAGCCCTTTGTCCTTGCCCGTGAC |
| | | TCTGGGTCAACCGGCGTCTATCAGCTGTAGATCTTCCCAAA |
| | | GCTTGGTCCACCGCAATGGCAATACATACCTTCATTGGTTT |
| | | CAGCAGCGGCCTGGGCAGTCTCCAAGGCGCTTGATATATA |
| | | CCGTCTCCAATCGGTTCTCCGGCGTTCCCGATAGATTCAGC |
| | | GGTTCAGGGAGTGGAACGGATTTTACGCTGAAAATAAGTA |
| | | GAGTCGAGGCAGAAGATGTCGGCGTCTACTACTGCTCCCA |
| | | ATCATCACATGTTCCTCCTACATTTGGTCAAGGGACAAGAC |
| | | TTGAGATTAAGGGCGGTGGAGGCTCAGGAGGGGGCGGAA |
| | | GCGGTGGGGGTGGATCTCAAGTGCAGCTCGTACAGAGCGG |
| | | ATCTGAACTCAAAAAGCCTGGGGCTAGCGTAAAAGTATCA |
| | | TGTAAAGCGAGCGGATATACCTTCACTAATTACGGCGTGA |
| | | ACTGGGTTCGCCAAGCGCCCGGACAAGGGCTCGAATGGAT |
| | | GGGGTGGATAAATCCCAACACGGGCGAACCTACCTACGAT |
| | | GACGACTTTAAAGGCCGGTTTGTGTTTAGTCTCGACACGAG |
| | | TGTCAGCACGGCTTACCTGCAGATTTCATCACTGAAGGCGG |
| | | AGGACACCGCTGTCTACTACTGCGCTAGAAGTAGAGGAAA |
| | | AAACGAAGCGTGGTTTGCCTATTGGGGTCAAGGTACGCTC |
| | | GTTACAGTCAGCTCT |
| 48 | 5311-04 | GACATTGTAGATACGCAATCACCATTGAGCCTGCCTGTCAC |
| | | ACTTGGTCAACCGGCATCTATTAGTTGTCGGAGTTCCCAGT |
| | | CCCTCGTCCATCGAAACGGAAATACGTATTTGCATTGGTTT |
| | | CAGCAGCGCCCTGGTCAGTCACCAAGACTGGGCATTCCCA |
| | | CAGTTAGTAACCGCTTTTCAGGAGTCCCCGACAGGTTCAGT |
| | | GGGAGTGGCTCCGGCACTGATTTTACGCTTAAGATAAGCC |
| | | GGGTTGAGGCCGAAGACGTAGGCGTCTATTACTGCTCTCA |
| | | GTCCTCACATGTTCCCCCAACATTCGGGCAGGGCACCCGAT |
| | | TGGAAATTAAGGGAGGTGGAGGCTCAGGCGGCGGAGGTTC |
| | | TGGGGGAGGCGGTTCCCAAGTTCAGCTTGTGCAGAGTGGA |
| | | TCAGAGCTGAAGAAGCCGGGGGCCTCTGTTAAAGTGTCCT |
| | | GTAAAGCATCAGGGTACACCTTCACTAACTATGGAGTAAA |
| | | CTGGGTGCGACAGGCGCCTGGGCAAGGCTTGGAATGGATG |
| | | GGATGGATCAATCCTAATACCGGGGAACCAACCTACGATG |
| | | ATGACTTTAAGGGGCGCTTCGTCTTCAGCTTGGACACGTCA |
| | | GTTAGCACGGCGGGGTTGCAAATATCATCACTCAAGGCAG |
| | | ATGATACAGCTGTTTATTTTTGTGCTCGAAGTAGAGGCAAA |
| | | AACGAAGCCTGGTTCGCATATTGGGGACAAGGTACACTTG |
| | | TGACGGTCTCCTCA |
| 49 | 5311-05 | GATGTGGTAATGACTCAAAGTCCGCTGAGCCTGCCCGTAA |
| | | CGTTGGGGCAACCCGCTTCAATCTCCTGTAGATCAAGCCAG |
| | | TCACTGGTCCACAGAAATGGCAATACATATTTGCACTGGTT |
| | | CCAACAGCGGCCAGGACAGTCTCCCAGACGACTCCCTTAT |
| | | ACTGTCTCTAATCGGTTTTCTGGCGTTCCGGATAGATTTAG |
| | | TGGCAGCGGCAGCGGGACCGACTTTACCCTGAAGATTTCA |
| | | CGAGTTGAGGCTGAAGATGTAGGAGTGTATTATTGCAGTC |
| | | AAAGCAGTCACGTTCCACCAACTTTCGGCCAGGGTACGAG |
| | | GCTGGAAATTAAGGGAGGCGGTGGGTCAGGGGGAGGCGG |
| | | ATCTGGAGGCGGCGGAAGTCAAGTGCAGCTTGTTCAGTCA |
| | | GGGAGCGAACTCAAGAAACCCGGAGCGTCTGTTAAAGTCA |
| | | GTTGTAAGGCATCCGGTTATACCTTCACCAACTATGGGGTA |
| | | AACTGGGTAAGACAAGCGCCAGGGCAAGGTCTGGAGTGG |
| | | ATGGGCTGGATCAACCCTAATACCGGTGAACCAACTTATG |
| | | ATGATGACTTCAAAGGGAGGTTTGTGTTCAGTCTCGACACG |
| | | TCAGTCTCCACTGCTGGGCTGCAAATCTCCAGTTTGAAGGC |
| | | AGAAGACACAGCCGTCTATTATTGCGCCCGCAGCCGCGGC |
| | | AAGAATGAGGCGTGGTTCGCATACTGGGGGCAGGGAACGC |
| | | TTGTCACTGTGAGCAGT |
| 50 | 543 | GATATACAAATGACCCAGTCACCTTCAAGCTTGTCAGCTTC |
| | | CGTCGGCGACAGGGTTACGATCACTTGTAGAGCCTCTCAG |
| | | GGTATAAGTTCTTGGCTGGCCTGGTACCAACAAAAACCCG |
| | | AAAAGGCCCCGAAAAGCCTGATATACGCGGCGAGTTCTCT |
| | | GCAGAGCGGAGTTCCCTCTAGGTTTTCAGGGTCTGGGTCAG |
| | | GAACAGATTTCACGCTCACAATCAGTTCTTTGCAGCCGGAG |
| | | GACTTCGCGACCTATTATTGCCAACAATACAACTCCTACCC |
| | | GTTGACATTCGGAGGAGGGACGAAAGTTGAAATCAAAGGC |
| | | GGGGGAGGGTCTGGGGGAGGCGGCTCTGGTGGGGGTGGCT |
| | | CTCAAGTCCAACTGGTTGAATCTGGGGGAGGCGTCGTTCA |
| | | GCCAGGGCGATCCCTGAGACTCTCCTGCGCTGCATCTGGTT |
| | | TCACGTTCTCATCATACGGAATGCACTGGGTAAGGCAGGC |
| | | GCCAGACAAGGGGCTGGAATGGGTTGCTGTTATTTGGTAT |
| | | GATGGCTCCAACAAGTATTATGCTGATTCTGTTAAGGGTAG |
| | | GTTCACCATTAGCCGAGATAACTCAAAGAACACTCTCTATC |
| | | TTCAAATGAATAGCCTTCGCGCAGAAGACACGGCAGTTTA |
| | | TTACTGTGCAAGGGGAGGAACTCTTGTCCGAGGGGCAATG |
| | | TATGGTACGGATGTCTGGGGTCAAGGGACCACGGTTACAG |
| | | TGTCATCT |
| 51 | 543-W | GATATTCAAATGACTCAGTCTCCGAGCTCACTGTCTGCTTC |
| | | AGTTGGGGACAGAGTTACTATAACGTGCCGCGCATCCCAG |
| | | GGAATTAGCAGTTGGCTGGCCTGGTACCAACAAAAGCCAG |
| | | GCAAAGCCCCGAAGCTGTTGATATATGCAGCTTCTTCTTTG |
| | | CAAAGTGGGGTACCATCTCGATTCTCCGGCTCAGGGTCTGG |
| | | AACAGACTTTACCCTGACGATTAGCAGTCTCCAGCCTGAA |
| | | GACTTTGCGACTTACTATTGTCAGCAATATAATTCCTATCC |
| | | ACTGACCTTCGGCGGGGGGACCAAAGTCGAAATTAAAGGG |
| | | GGCGGAGGCTCCGGGGGAGGTGGGTCTGGGGGAGGAGGG |
| | | AGTCAAGTTCAACTCGTAGAGTCCGGAGGCGGTGTGGTAC |
| | | AACCTGGTAGGTCCCTGCGACTCTCTTGTGCAGCTAGCGGT |
| | | TTTACCTTTTCTTCTTATGGTATGCACTGGGTTAGGCAAGCT |
| | | CCCGGGAAGGGCCTGGAATGGGTCGCAGTGATTTGGTACG |
| | | ACGGAAGTAACAAGTACTATGCTGACTCAGTTAAGGGCCG |
| | | ATTTACCATCTCACGAGATAATTCCAAAAATACACTGTACC |
| | | TCCAAATGAATAGCCTCCGGGCGGAGGACACGGCTGTGTA |
| | | TTATTGTGCGCGAGGTGGTACGTTGGTGCGCGGAGCTATGT |
| | | ATGGTACTGACGTTTGGGGTCAGGGTACGACCGTCACGGT |
| | | AAGCTCT |
| 52 | 553 | GATATCCAACTCACGCAGAGCCCCCTGTCCCTTCCGGTGAC |
| | | CCTGGGGCAACCCGCTAGTATTTCTTGTCGATCCTCCCAAT |
| | | CTCTTGTTCACCGAAACGGTAACACCTACCTGCACTGGTTT |
| | | CAACAACGGCCAGGACAGAGCCCCAGGCTTTTGATATATA |
| | | CGGTCTCAAATAGATTCAGTGGAGTCCCTGACCGATTTTCC |
| | | GGCAGCGGCTCAGGCACGGATTTTACGCTGAAGATAAGCC |
| | | GGGTCGAAGCGGAAGACGTTGGTGTTTACTTTTGCTCTCAA |
| | | TCCTCTCACGTTCCACCGACCTTCGGCGCCGGCACACGCCT |
| | | TGAGATAAAAGGGGGCGGAGGAAGCGGTGGGGGAGGGAG |
| | | TGGAGGAGGGGGGTCACAAGTCCAACTGCAAGAATCAGGT |
| | | GGTGGATCCGTCCAAGCTGGAGGTTCTCTTCGACTTAGTTG |
| | | CGTGGCTTCTGGTCTGGTGTACTCATTGAAAGCATACGATT |
| | | GGGGCTGGGCCCGACAAGCTCCTGGCAAAGAATGTGAGCT |
| | | GGTGGGTCGGATTATGCCGAACGGCGCTACTGTTTACATTG |
| | | ATTCCGTTAAAGGTCGGTTCATCATTTCCCGCGAAAATTCT |
| | | AATATGACTGTATACCTTCAAATGAATAGCCTTAAATTCGA |
| | | GGACACGGCAGTCTATTACTGCGCCAGGGTAGGGGACCCA |
| | | AGCGGAGGATGGTGTAAAGGACAAGGTACTCAAGTTACAG |
| | | TAAGTTCT |
| 53 | 553-W | GACATTGTTGATACTCAATCTCCTCTCTCTTTGCCAGTCACC |
| | | CTTGGGCAGCCAGCTTCTATCTCCTGTCGCTCAAGCCAGTC |
| | | TCTCGTGCACAGGAATGGCAATACGTACCTGCATTGGTTCC |
| | | AACAAAGGCCCGGTCAGTCACCCCGCCTGGGCATTCCCAC |
| | | CGTATCAAATAGGTTTTCTGGTGTACCCGACAGGTTTTCCG |
| | | GGAGCGGAAGCGGCACTGACTTCACACTCAAAATATCAAG |
| | | GGTCGAAGCGGAGGATGTTGGTGTTTACTACTGTAGTCAGT |
| | | CTAGTCATGTGCCGCCCACCTTCGGCCAGGGCACGCGACT |
| | | GGAAATCAAGGGAGGGGGTGGCTCTGGAGGCGGCGGAAG |
| | | CGGAGGTGGTGGATCCCAGGTTCAACTCGTAGAGTCAGGA |
| | | GGAGGACTCGTCCAACCCGGTGGGAGCCTCCGACTTAGCT |
| | | GCGTGGCGTCTGGACTTACTTTCAGTCTGAAGGCGTATGAT |
| | | TGGGGATGGATTCGACAAGCCCCTGGTAAGGCCCTCGAAT |
| | | GGGTCGGACGGATAATGCCCAACGGAGCGACGGTTTACAT |
| | | CGACTCTGTGAAAGGAAGATTTACTATCTCACGAGACAAC |
| | | AGCAAAAACACGGTTTATCTTCAGATGAATTCACTCAAGG |
| | | CAGAGGATACTGCTGTATACTATTGTGCGCGCGTGGGGGA |
| | | CCCTTCAGGCGGGTGGTGTTGGGGCCAAGGCACGCTCGTT |
| | | ACTGTATCAAGC |
| 54 | 563 | GACATCCAGATGACCCAATCCCCAAGTTCAGTTTCAGCTTC |
| | | TGTTGGTGACCGAGTCACAATTACTTGTCGGGCGTCACAAG |
| | | GAATCAGTAGTTGGGTCGCGTGGTATCAACAAAAACCCGG |
| | | GAAAGCGCCGAAACTCCTTATATATGGATCCTCAAGGTTG |
| | | CAATCTGGAGTACCTTCCCGCTTTAGCGGCTCAGGATCCGG |
| | | GACCGATTTCACACTCACCATTTCTTCACTTCAACCTGAGG |
| | | ATTTCGCCACTTACTATTGTCAACAGTACAATACTTACCCA |
| | | TTGACTTTTGGCGGAGGCACAAAAGTAGAGATCAAGGGCG |
| | | GTGGAGGTTCCGGAGGTGGAGGGTCTGGCGGAGGGGGGTC |
| | | TCAAGTACAGCTCGTTGAGTCAGGTGGTGGTGTAGTCCAG |
| | | CCAGGCCGGAGTTTGCGATTGAGTTGCGCTGCCTCTGGTTT |
| | | TAATTTCTCTGACTACGGGATGCATTGGGTCCGCCAGGCCC |
| | | CTGACAAAGGTCTCGAGTGGGTTGCAGTGATCTATGATGG |
| | | TTCAATCTCATACTATGCGGATTCAGTAAAGGGACGGTTTA |
| | | CGATCTCCAGAGACAACTCAAAGAATACGCTCTATCTCCA |
| | | GATGAACAGCCTCCGCGCAGAAGATACAGCCGTGTATTAT |
| | | TGTGCAAGGGGAGGAACCGTTGAACATGGAGCGGTATATG |
| | | GCACTGATGTCTGGGGTCAGGGGGCTACAGTTACGGTATC |
| | | ATCA |
| 55 | 563-W | GACATACAAATGACTCAGAGCCCCTCCTCTGTGTCAGCCTC |
| | | AGTAGGTGATAGGGTAACCATTACCTGTCGAGCATCCCAA |
| | | GGTATTTCATCCTGGGTCGCATGGTATCAGCAAAAACCCG |
| | | GGAAAGCACCTAAACTCCTGATCTACGGTTCTTCCCGGCTT |
| | | CAATCCGGTGTTCCTAGCCGGTTCTCAGGGAGCGGATCTGG |
| | | AACAGACTTTACCTTGACGATTAGCTCTCTCCAGCCCGAGG |
| | | ATTTTGCAACCTACTACTGCCAACAATATAATACATACCCA |
| | | TTGACGTTTGGAGGAGGCACTAAAGTAGAGATTAAAGGGG |
| | | GAGGGGGTTCAGGAGGAGGTGGTTCCGGTGGGGGTGGCAG |
| | | TCAAGTCCAGCTTGTAGAGTCTGGTGGCGGGGTAGTACAG |
| | | CCAGGTCGATCTTTGCGCTTGAGCTGCGCCGCCTCCGGTTT |
| | | TACTTTCAGTGACTATGGAATGCATTGGGTTCGGCAAGCTC |
| | | CCGGGAAAGGCCTTGAGTGGGTGGCGGTCATTGATGACGG |
| | | AAGTATTTCCTACTACGCGGATAGTGTTAAGGGAAGGTTTA |
| | | CCATATCAAGGGACAATTCCAAAAATACACTTCCTCTTCAA |
| | | ATGAATTCACTCCGGGCTGAGGACACTGCCGTCTACTATTG |
| | | TGCTCGAGGGGGTACAGTAGAGCATGGAGCCGTTTACGGT |
| | | ACTGACGTATGGGGTCAAGGTACAACAGTAACTGTAAGCT |
| | | CA |
| 56 | 5321 | GACATCCAGATGACTCAAACGACAAGCTCCTTGTCTGCCA |
| | | GCCTTGGTGATCGGGTTACTATATCCTGTCGGGCCAGTCAG |
| | | TCCCTTGTACATCGCAATGGCAATACTTATCTTAACTGGTA |
| | | TCAGCAAAAACCCGATGGGACCGTTAAGCTTCTCATCTATA |
| | | CTGTATCTCGATTGCATTCAGGGGTTCCTTCCCGATTTAGT |
| | | GGAAGCGGTAGTGGAACGGATTATAGTCTGACAATTTCCA |
| | | ATCTTGAACAAGAAGACATAGCAACTTACTTTTGCTCCCAA |
| | | TCCTCACACGTCCCCCCAACTTTTGGAGGAGGTACCAAGCT |
| | | GGAGATCACAGGGGGCGGAGGGTCAGGTGGAGGTGGAAG |
| | | TGGAGGTGGTGGTTCAGAGGTGAAGTTGCAAGAATCCGGT |
| | | CCGGGACTCGTGGCCCCTAGCCAATCCTTGAGTGTCACGTG |
| | | TACAGTCTCAGGTTATACTTTTACAAATTACGGGGTGAGCT |
| | | GGATACGGCAGCCACCTAGGAAAGGACTCGAATGGCTCGG |
| | | AGTCATCAACCCCAACACGGGCGAACCGTATTATAACTCT |
| | | GCCCTCAAAAGTCGGCTTACAATAATCAAAGACAACAGCA |
| | | AAAGCCAAGTGTTTTTGAAGATGAACTCCCTCCAGACCGA |
| | | CGATACTGCAATTTATTATTGTTCCAGATGTCGGGGAAAGA |
| | | ACGAGGCCTGGTTCGCGTACTGGGGTCAAGGTACCTCCGT |
| | | AACCGTTTCCAGC |
| 57 | 534 | GACATCCAGATGACGCAGACGACGAGTAGCCTGTCTGCGT |
| | | CTCTTGGTGATCGAGTCACTATAAGTTGCCGAGCATCTCAA |
| | | TCCCTCGTTCATCGCAATGGGAACACTTACCTCAACTGGTA |
| | | TCAACAGAAGCCTGACGGTACGGTCAAGCTGCTGATCTAT |
| | | ACGGTGTCAAGGCTCCACTCTGGAGTTCCCAGCAGGTTTTC |
| | | CGGAAGCGGGAGCGGTACAGATTATTCTCTCACTATTTCTA |
| | | ATCTGGAGCAAGAAGACATAGCAACCTATTTCTGCTCACA |
| | | GTCCTCTCACGTACCCCCTACTTTCGGCGGAGGTACAAAAC |
| | | TGGAGATTACAGGGGGTGGTGGTTCCGGAGGAGGAGGTAG |
| | | CGGGGGAGGAGGGAGCCAGGTACAGCTCCAGCAGTCTGGC |
| | | AGTGAGCTTAAAAAACCAGGGGCTTCTGTCAAAGTCTCCT |
| | | GTAAAGCGAGCGGTTATACCTTCACCAACTATAATGTAAA |
| | | CTGGATAAAACAGGCGCCCGGGCAAGGGCTCCAGTGGATG |
| | | GGATGGATCAATCCAAACACGGGCGAGCCTACCGCTGATG |
| | | ACGATTTTAAGGGCCGATTTGCTTTCTCATTGGACACGTCC |
| | | GTTAGCACCGCATATCTCCAAATATCAAGCCTCAAGGCGG |
| | | ACGACACGGCTGTTTATTTTTGTAGTAGGTCTAGGGGGAAA |
| | | AACGAAGCGTGGTTTGCGTATTGGGGCCAGGGTACACTGG |
| | | TGACTGTCTCTTCA |
| 58 | 532 | GATATTCAGATGACTCAAACTACTTCCAGCCTCTCAGCGTC |
| | | TCTGGGTGACCGAGTCACAATTAGTTGCAGAGCGTCTCAGT |
| | | CTCTGGTTCACCGCAACGGGAACACGTATTTGAATTGGTAT |
| | | CAACAAAAACCGGATGGCACAGTGAAACTTCTCATCTACA |
| | | CGGTTAGCAGATTGCACTCAGGCGTCCCAAGTCGATTCTCC |
| | | GGCAGCGGTAGTGGAACAGATTATAGCCTTACAATCTCCA |
| | | ATCTGGAACAAGAAGACATCGCGACTTATTTTTGTTCCCAG |
| | | TCTTCCCACGTTCCGCCTACCTTCGGCGGGGGCACCAAACT |
| | | GGAGATTACCGGGGGAGGTGGTAGTGGTGGCGGTGGAAGC |
| | | GGAGGGGGCGGCAGTCAAGTGCAGTTGCAACAGTCTGGTT |
| | | CCGAACTGAAAAAGCCGGGGGCCTCAGTGAAAGTCTCTTG |
| | | CAAAGCATCTGGCTATACTTTCACTAATTATGGGATGAACT |
| | | GGATAAAACAAGCCCCCGGGCAAGGCCTTCAATGGATGGG |
| | | GTGGATAAATCCAAATACAGGAGAGCCGACGGCGGATGAT |
| | | GATTTTAAGGGCAGGTTCGCGTTCAGCCTGGACACTAGCGT |
| | | ATCAACAGCATATCTCCAAATAAGCAGTCTTAAGGCGGAC |
| | | GATACCGCCGTTTATTTCTGCAGCCGAAGCCGCGGGAAAA |
| | | ACGAAGCCTGGTTCGCGTATTGGGGGCAGGGCACACTCGT |
| | | GACCGTTAGTAGC |
| 59 | 5310 | GATATACAGATGACGCAAACTACCAGCTCATTGTCAGCGT |
| | | CCCTTGGTGACCGCGTTACAATTAGTTGTAGGGCCTCCCAA |
| | | TCTCTCGTCCACCGGAACGGTAATACCTATCTGAATTGGTA |
| | | CCAGCAGAAACCGGATGGTACCGTTAAGCTCTTGATATAT |
| | | ACAGTGAGCAGATTGCACAGTGGCGTTCCGTCTAGATTCA |
| | | GTGGATCAGGTTCCGGCACCGATTACTCCCTGACAATTTCT |
| | | AATTTGGAGCAAGAAGATATCGCAACGTACTTTTGTAGCC |
| | | AATCCTCTCATGTCCCGCCTACGTTTGGCGGCGGCACTAAA |
| | | CTGGAGATAACCGGGGGCGGTGGCTCTGGAGGCGGTGGAT |
| | | CCGGCGGAGGGGGCAGTCAGGTTCAGTTGCAACAATCAGG |
| | | CTCTGAATTGAAGAAACCGGGTGCCTCTGTGAAGGTGTCCT |
| | | GCAAGGCGAGCGGGTATACATTTACTAACTACGGTGTCAA |
| | | TTGGATTAAGCAGGCTCCGGGCCAGGGATTGCAGTGGATG |
| | | GGGTGGATTAACCCAAACACAGGCGAGCCAACTTTCGACG |
| | | ACGACTTTTGGGGAAGATTCGCTTTTTCCCTGGACACTAGC |
| | | GTGTCTACCGCTTATCTCCAAATATCAAGCCTGAAAGCGGA |
| | | TGATACTGCCGTATACTTCTGTAGCCGCAGTAGGGGCAAA |
| | | AACGAAGCGTGGTTCGCGTATTGGGGACAGGGAACTTTGG |
| | | TCACAGTCTCATCC |
| 60 | 5319 | GACATTCAGATGACCCAAACTACTTCCAGTCTTTCAGCCTC |
| | | CCTTGGTGATAGGGTGACGATTAGTTGCCGCGCTTCACAGT |
| | | CCCTGGTACACAGGAATGGCAATACTTATCTTAACTGGTAC |
| | | CAACAAAAACCCGACGGCACCGTCAAACTTCTCATCTATA |
| | | CGGTCTCTAGGTTGCATTCCGGAGTCCCATCACGGTTTAGT |
| | | GGTTCCGGCAGCGGTACAGACTATAGCCTGACTATCTCTAA |
| | | CCTTGAGCAAGAAGACATCGCCACCTACTTTTGCTCACAGA |
| | | GCTCCCACGTCCCTCCTACCTTCGGCGGTGGTACAAAACTG |
| | | GAGATCACCGGGGGAGGTGGATCTGGCGGGGGTGGATCTG |
| | | GTGGGGGCGGAAGTCAGGTGCAGTTGCAGCAATCCGGTTC |
| | | TGAACTTAAAAAGCCTGGGGCCAGTGTAAAAGTGTCATGT |
| | | AAGGCAAGCGGGTACACCTTTACAAATTATGGAGTGAACT |
| | | GGATAAAACAGGCCCCTGGGCAAGGATTGCAATGGATGGG |
| | | GTGGATAAATCCCAACACCGGAGAGCCGACATTCGATGAC |
| | | GACTTCTGGGGCAGATTTGCCTTCTCCCTCGATACGTCAGT |
| | | GTCCACTGCGTATCTTCAAATTTCCTCCTTGAAAGCCGATG |
| | | ATACTGCCGTGTATTTTTGCTCCCGGAGCCGGGAGAAAAA |
| | | CGAAGCCTGGTTCGCTTATTGGGGCCAGGGTACTTTGGTTA |
| | | CTGTAAGTTCT |
| 61 | 5315 | GATATCCAAATGACACAGACCACCTCATCTCTTAGCGCCTC |
| | | TCTGGGCGATCGAGTCACGATTTCCTGTCGGGCGTCTCAGT |
| | | CATTGGTACACAGGAATGGTAATACCTATTTGAATTGGTAC |
| | | CAACAAAAACCCGATGGCACGGTAAAGCTGCTGATTTATA |
| | | CTGTCTCCAGGCTGCACTCCGGAGTCCCGTCTCGGTTCAGC |
| | | GGTAGTGGGAGCGGCACTGACTACTCACTTACAATCTCCA |
| | | ACCTTGAACAAGAGGATATAGCTACATATTTCTGTAGTCAG |
| | | AGCAGCCATGTCCCACCTACCTTCGGTGGAGGCACGAAAT |
| | | TGGAAATTACAGGTGGTGGTGGCTCTGGTGGCGGAGGGTC |
| | | TGGTGGAGGCGGATCTCAGGTCCAGCTTCAACAGAGTGGT |
| | | AGCGAGCTGAAGAAACCCGGCGCTTCAGTAAAGGTCTCAT |
| | | GCAAGGCATCCGGGTACACGTTTACAAATTATGGCGTTAA |
| | | TTGGATCAAACAAGCGCCCGGTCAAGGCCTGCAATGGATG |
| | | GGGTGGATCAACCCAAATACTGGCGAGCCAACTTTCGACG |
| | | ATGATTTTTGGGGGCGATTCGCTTTTAGCCTTGACACATCC |
| | | GTATCTACGGCTTATCTCCAGATTTCAAGCCTCAAGGCCGA |
| | | TGATACCGCAGTATATTTTTGCTGTCGCTGCCGCGAAAAGA |
| | | ATGAAGCCTGGTTCGCCTATTGGGGGCAGGGCACCCTTGTC |
| | | ACTGTGTCATCA |
| 62 | 57 | GACATCCAGTTGACACAAAGCCCATTGAGTCTGCCGGTCA |
| | | CACTGGGTCAGCCGGCCTCCATTTCATGCAGGTCATCACAA |
| | | AGTGAGGTACACAGGAATGGTAACACATACCTTCATTGGT |
| | | TCCAGCAAAGACCTGGTCAATCTCCCCGATTGCTTATTTAC |
| | | ACAGTCAGTAACCGCTTTAGTGGCGTCCCAGATCGCTTCTC |
| | | AGGCTCTGGTTCCGGCACGGATTTCACGCTGAAAATAAGC |
| | | AGGGTTGAGGCCGAGGATGTGGGGGTTTATTTTTGCAGCC |
| | | AGTCCAGTCATGTCCCACCAACCTTCGGGGCCGGAACTCG |
| | | GTTGGAGATTAAAGGCGGTGGGGGGTCTGGCGGAGGGGGT |
| | | AGCGGCGGAGGCGGTTCCCAGGTTCAGCTCCAACAAAGCG |
| | | GAAGTGAACTGAAGAAACCAGGAGCATCCGTGAAAGTTAG |
| | | CTGTAAAGCGTCAGGATATACGTTTACGAATTATGGCGTG |
| | | AATTGGATCAAACAAGCCCCCGGGCAAGGGCTCCAATGGA |
| | | TGGGATGGATAAACCCTAACACCGGGGAACCAACTTTTGA |
| | | TGACGATTTCAAGGGAAGGTTTGCCTTCTCTTTGGATACTT |
| | | CTGTTAGTACAGCTTACCTTCAAATTAGCAGCTTGAAGGCG |
| | | GACGACACAGCAGTCTACTTCTGTAGTCGGTCTAGAGGGA |
| | | AAAATGAAGCGTGGTTTGCATATTGGGGGCAGGGCACATT |
| | | GGTGACAGTTAGTTCT |
| 63 | 573 | GATATACAGCTTACCCAATCACCATTGTCCCTCCCAGTTAC |
| | | CTTGGGTCAGCCAGCATCTATATCTTGCAGGAGCAGCCAAT |
| | | CATTGTGGCACCGGAACGGAAATACCTACCTTCATTGGTTT |
| | | CAGCAAAGGCCGGGACAGTCACCGCGACTGTTGATATACA |
| | | CCGTCTCTAATCGATTTTCCGGTGTACCGGATCGATTTAGC |
| | | GGGTCTGGATCTGGTACAGATTTTACACTTAAGATAAGCCG |
| | | AGTAGAAGCGGAAGATGTAGGAGTGTATTTTTGCAGTCAG |
| | | AGTAGCCACGTTCCGCCAACGTTCGGAGCTGGAACGAGGT |
| | | TGGAGATAAAAGGGGGCGGTGGAAGCGGCGGTGGCGGCA |
| | | GTGGTGGAGGTGGTTCTCAGGTCCAATTGCAGCAGTCTGG |
| | | CTCCGAACTCAAGAAGCCGGGAGCCAGCGTGAAAGTAAGC |
| | | TGCAAAGCGAGCGGATATACGTTTACCAATTATGGGGTAA |
| | | ATTGGATAAAACAAGCTCCCGGTCAAGGGTTGCAATGGAT |
| | | GGGGTGGATAAACCCGAATACAGGCGAACCGACATTTGAT |
| | | GATGACTTCAAGGGTCGATTCGCCTTTAGCTTGGACACATC |
| | | AGTGTCTACGGCCTATCTTCAGATCAGTTCCCTCAAAGCAG |
| | | ATGATACCGCCGTGTATTTTTGTTCTCGGTCACGCGGTAAA |
| | | AATGAGGCATGGTTCGCTTACTGGGGGCAAGGGACGCTTG |
| | | TCACAGTCAGTTCA |
| 64 | 576 | GACATACAACTTACCCAGTCTCCGCTCAGCCTCCCAGTAAC |
| | | CCTGGGACAACCTGCGAGCATCTCCTGTAGGTCTAGCCAGT |
| | | CCTTGGTCCATCGCAACGGAAACACCTACTGTCATTGGTTT |
| | | CAGCAAAGGCCTGGTCAGTCCCCCAGGCTGCTTATCTATAC |
| | | GGTTAGCAACCGGTTTAGTGGAGTCCCCGATCGCTTCTCCG |
| | | GTAGCGGAAGTGGGACTGACTTTACTCTGAAAATTTCACG |
| | | AGTGGAGGCCGAAGATGTTGGGGTTTATTTTTGCAGTCAGT |
| | | CTAGTCATGTTCCACCAACCTTTGGTGCCGGAACACGGCTC |
| | | GAAATCAAAGGAGGTGGCGGCAGCGGAGGTGGCGGCTCA |
| | | GGGGGCGGCGGGTCTCAAGTGCAACTGCAGCAATCTGGTT |
| | | CTGAGCTGAAGAAACCTGGAGCCTCAGTTAAAGTCTCCTG |
| | | TAAGGCTTCTGGGTATACATTTACAAACTATGGGGTAAATT |
| | | GGATAAAACAGGCTCCGGGCCAAGGTCTTCAGTGGATGGG |
| | | TTGGATCAATCCCAACACCGGTGAACCTACTTTCGATGATG |
| | | ATTTCAAAGGACGGTTTGCATTCTCTCTTGACACTTCCGTG |
| | | AGCACAGCTTACCTCCAAATTTCCTCCCTTAAAGCTGATGA |
| | | TACAGCCGTGTATTTTTGTTCCCGGAGCCGGGGTAAGAACG |
| | | AAGCCTGGTTCGCATATTGGGGTCAAGGCACACTTGTAAC |
| | | AGTTAGTAGT |
| 65 | 578 | GACATCCAGTTGACGCAGTCTCCATTGTCCCTGCCCGTAAC |
| | | CTTGGGACAACCTGCGTCAATTAGCTGCCGATCAAGCCAA |
| | | TCCCTTGTTCACCGGAACGGGAACACGTATCTTCACTGGTT |
| | | CCAGCAACGCCCTGGGCAGAGTCCGCGGTTGCTGATCTAT |
| | | GAGGTCAGCAACCGATTTTCTGGCGTTCCGGACCGCTTCTC |
| | | CGGCAGTGGGAGTGGCACGGACTTCACCCTCAAGATCAGC |
| | | CGGGTCGAAGCCGAGGACGTGGGCGTATATTTCTGCTCTC |
| | | AAAGCTCTCATGTTCCACCTACGTTTGGCGCAGGGACGAG |
| | | GCTCGAGATCAAAGGGGGTGGGGGCTCAGGCGGTGGCGG |
| | | AAGCGGCGGAGGCGGCAGTCAAGTCCAGTTGCAGCAATCA |
| | | GGTAGCGAACTCAAAAAGCCCGGCGCAAGTGTTAAAGTAT |
| | | CCTGTAAGGCTAGCGGCTACACATTCACTAATTATGGGGTA |
| | | AACTGGATCAAACAAGCGCCCGGTCAAGGACTCCAGTGGA |
| | | TGGGCTGGATTAATCCCAACACAGGTGAACCGACTTTCGA |
| | | CGACGATTTTAAGGGAAGATTTGCATTCTCTCTCGATACCT |
| | | CAGTCTCCACCGCCTATCTCCAGATCAGTAGCCTTAAAGCC |
| | | GACGACACCGCTGTGTATTTCTGTTCCCGATCTCGAGGAAA |
| | | GAACGAAGCGTGGTTCGCCTATTGGGGGCAAGGTACGTTG |
| | | GTGACCGTATCCTCC |
| 66 | 579 | GATATCCAACTTACCCAATCTCCCCTCAGCTTGCCAGTCAC |
| | | GCTTGGCCAACCCGCCAGCATTTCCTGTAGGTCATCACAGA |
| | | GTCTCGTCCATAGGAATGGTAATACATACTTGCATTGGTTT |
| | | CAACAAAGACCCGGGCAAAGTCCACGGCTGCTCATATACA |
| | | CAGAGAGCAACAGATTTTCTGGGGTTCCGGATAGGTTTTCT |
| | | GGCTCTGGTAGTGGAACAGACTTTACTCTTAAGATCAGTAG |
| | | GGTCGAAGCTGAGGATGTAGGGGTTTACTTTTGCTCCCAGT |
| | | CCAGCCATGTCCCACCAACCTTTGGCGCAGGCACGAGATT |
| | | GGAGATTAAGGGCGGCGGGGGCAGCGGTGGAGGCGGGAG |
| | | CGGTGGAGGAGGTTCTCAGGTGCAGCTTCAACAGTCAGGA |
| | | TCAGAGCTCAAGAAGCCTGGAGCCAGCGTGAAAGTATCTT |
| | | GTAAGGCCTCCGGATACACCTTTACCAATTATGGGGTTAAT |
| | | TGGATAAAACAAGCGCCTGGGCAAGGGTTGCAATGGATGG |
| | | GATGGATAAACCCTAACACCGGAGAGCCGACTTTCGACGA |
| | | TGATTTCAAAGGTCGGTTTGCATTCTCCTTGGACACGTCCG |
| | | TATCAACAGCATATCTCCAAATTTCATCCCTCAAGGCAGAC |
| | | GACACGGCGGTGTATTTTTGTAGTAGGAGTCGAGGCAAGA |
| | | ACGAAGCATGGTTCGCATATTGGGGGCAAGGCACGCTTGT |
| | | TACGGTGAGCTCT |
| 67 | 5711 | GACATCCAGCTTACCCAGAGTCCACTGAGCCTTCCCGTAAC |
| | | GCTCGGTCAACCCGCAAGTATATCTTGCCGCTCTAGTCAAA |
| | | GTCTGGTCCATCGGAACGGTAATACATATTTGCACTGGTTT |
| | | CAACAGAGACCAGGGCAAAGTCCCAGGCTCTTGATCTACA |
| | | CTGTAAGTAACAGATTCAGCGGTGTTCCGGACAGGTTCAG |
| | | CGGCAGTGGCTCCGGTACTGATTTCACCCTGAAAATCAGTA |
| | | GAGTTGAAGCGGAGGACGTAGGCGTATACTTCTGCAGCTG |
| | | TAGCAGTCACGTACCTCCTACGTTTGGAGCCGGCACGCGG |
| | | CTCGAGATTAAAGGAGGCGGCGGCTCCGGTGGTGGTGGCA |
| | | GCGGTGGAGGGGGGAGTCAAGTGCAACTCCAACAGTCAGG |
| | | GAGCGAGCTCAAAAAACCTGGGGCAAGCGTCAAAGTATCC |
| | | TGCAAAGCAAGTGGGTACACATTCACCAACTATGGGGTCA |
| | | ACTGGATTAAACAAGCTCCGGGGCAGGGTCTTCAGTGGAT |
| | | GGGATGGATAAACCCGAATACTGGGGAGCCCACTTTCGAC |
| | | GACGACTTCAAAGGGCGATTTGCCTTCTCCCTCGACACCAG |
| | | CGTTAGTACGGCGTATTTGCAAATATCATCCCTCAAAGCAG |
| | | ATGATACCGCTGTGTATTTCTGTTCCAGAAGCAGGGGAAA |
| | | GAACGAGGCTTGGTTTGCATATTGGGGTCAAGGCACGCTC |
| | | GTTACAGTCAGTTCC |
| 68 | 5718 | GATATACAGCTTACCCAGTCCCCCCTGAGTTTGCCTGTCAC |
| | | TTTGGGTCAGCCGGCTTCAATCTCATGCAGGTCTTCTCAGT |
| | | CTCTGGTTCACCGGAATGGTAACACTTATCTTCATTGGTTT |
| | | CAGCAGCGGCCTGGTCAGTCACCGAGATTGTTGATTTATAC |
| | | CGTGTCAAACAGGTTTTCAGGTGTACCTGACAGGTTTTCAG |
| | | GAAGTGGCTCTGGAACTGACTTTACATTGAAAATATCCCGC |
| | | GTTGAGGCGGAAGATGTGGGGGTGTACTTTTGTAGTCAGA |
| | | GTTCTCATTGGCCCCCCACATTCGGCGCGGGCACACGATTG |
| | | GAGATAAAAGGCGGGGGAGGCAGCGGTGGGGGAGGATCA |
| | | GGTGGAGGTGGGAGTCAGGTTCAGCTGCAACAATCAGGGA |
| | | GTGAGTTGAAGAAACCAGGCGCTTCCGTCAAGGTCAGCTG |
| | | TAAAGCGTCAGGATATACTTTTACAAACTACGGAGTCAATT |
| | | GGATTAAGCAGGCTCCTGGTCAGGGGTTGCAATGGATGGG |
| | | ATGGATAAATCCAAACACGGGTGAACCTACATTCGACGAC |
| | | GACTTCAAGGGACGATTCGCATTTTCACTTGACACGAGTGT |
| | | GAGCACAGCCTATCTCCAAATCTCCTCACTGAAGGCGGAT |
| | | GATACTGCTGTGTATTTCTGCTCACGATCCAGGGGTAAAAA |
| | | CGAAGCATGGTTTGCGTACTGGGGGCAGGGCACACTTGTC |
| | | ACGGTATCATCC |
| 69 | 5721 | GACATCCAACTGACACAATCCCCACTGAGTTTGCCAGTCAC |
| | | GCTGGGACAACCCGCATCTATATCCTGCCGGTCCTCACAAA |
| | | GTTTGGTACATCGCAACGGTAATACTTACTTGCATTGGTTT |
| | | CAACAACGACCGGGGCAATCACCCAGACTTCTCATATACA |
| | | CGGTAAGCAACCGATTTAGTGGAGTACCTGACAGATTCTCT |
| | | GGCTCTGGTAGCGGGACCGATTTCACTCTCAAAATAAGCA |
| | | GAGTAGAAGCGGAGGATGTGGGCGTATATTTTTGTAGTCA |
| | | ATCTAGTCATGTTCCACCTTGCTTTGGAGCCGGTACTCGAC |
| | | TCGAAATTAAAGGTGGAGGGGGTTCTGGAGGAGGCGGAA |
| | | GCGGAGGAGGTGGTTCCCAAGTTCAGCTGCAACAAAGTGG |
| | | AAGTGAATTGAAGAAGCCTGGTGCATCTGTCAAAGTGTCC |
| | | TGCAAAGCCTCTGGCTACACATTCACGAACTATGGCGTTAA |
| | | TTGGATCAAGCAGGCGCCGGGTCAAGGCCTTCAATGGATG |
| | | GGGTGGATAAATCCGAATACGGGGGAACCCACGTTTGACG |
| | | ATGACTTCAAGGGTAGGTTTGCTTTTTCTCTTGACACCTCC |
| | | GTTTCTACTGCCTATCTTCAAATAAGCTCACTCAAAGCTGA |
| | | TGACACAGCCGTTTATTTCTGCAGTCGCAGTCGAGGGAAA |
| | | AACGAGGCGTGGTTTGCGTATTGGGGCCAGGGCACCCTTG |
| | | TAACGGTGTCCAGT |
| 70 | 532 | GATATACAGCTGACGCAATCTCCGCTGAGTCTCCCCGTTAC |
| | | TCTGGGCCAACCTGCATCCATTAGTTGTCGCTCCAGTCAGT |
| | | CACTTGTACACCGAAATGGAAACACCTACTTGCACTGGTTC |
| | | CAGCAAAGACCCGGCCAGTCCCCACGCCTTCTCATATATAC |
| | | TGTAAGTAATCGCTTTTCAGGTGTGCCTGATAGGTTTAGCG |
| | | GGAGCGGGAGCGGCACGGACTTCACACTTAAAATTTCTAG |
| | | AGTAGAGGCCGAGGACGTAGGTGTGTACTTTTGTTCCCAGT |
| | | CTAGCCACGTCCCACCTACCTTTGGGGCGGGGACCAGATT |
| | | GGAGATTAAGGGCGGCGGAGGGAGTGGTGGAGGGGGCTC |
| | | AGGAGGAGGCGGCAGCCAAGTTCAACTGCAGCAGAGTGGT |
| | | TCCGAGCTCAAGAAGCCGGGCGCTTCTGTTAAGGTCTCATG |
| | | TAAAGCTTCCGGTTATACATTTACGAACTATGGTATGAATT |
| | | GGATCAAGCAAGCACCCGGACAGGGATTGCAATGGATGGG |
| | | CTGGATAAATCCCAATACAGGTGAGCCCACGTTCGATGAC |
| | | GACTTCAAAGGTCGGTTTGCCTTTTCCCTGGACACATCCGT |
| | | CAGTACCGCGTACCTTCAGATTTCTAGCCTCAAGGCCGACG |
| | | ATACCGCGGTCTACTTCTGTTCCCGCTCTCGCGGAAAGAAT |
| | | GAGGCCTGGTTTGCATACTGGGGTCAGGGAACACTCGTTA |
| | | CCGTTTCCTCT |
| 71 | 536 | GACATTCAACTCACACAGAGCCCGCTGTCTCTCCCAGTCAC |
| | | GCTGGGTCAACCAGCATCAATCTCATGTAGATCAAGCCAA |
| | | TCACTGGTGCATAGGAATGGAAACACGTACCTTCACTGGTT |
| | | CCAACAGAGGCCCGGGCAAAGCCCTCGGCTGCTTATATAT |
| | | ACTGTATCAAATCGGTTCAGCGGAGTACCGGACCGATTTA |
| | | GTGGATCCGGGAGCGGGACCGACTTCACCCTCAAAATCTC |
| | | CCGAGTCGAAGCCGAAGACGTAGGTGTTTACTTCTGCTCAC |
| | | AGTCAAGTCACGTCCCCCCGACGTTTGGCGCGGGTACACG |
| | | ACTCGAGATTAAGGGTGGAGGCGGAAGCGGCGGTGGAGG |
| | | GAGCGGCGGTGGCGGCAGTCAAGTTCAATTGCAACAGAGT |
| | | GGATCCGAGCTGAAAAAGCCTGGGGCTTCAGTAAAAGTTA |
| | | GTTGTAAGGCAAGTGGGTATACTTTTACTAATTACTGGGTG |
| | | AATTGGATAAAACAAGCCCCGGGCCAAGGCCTTCAGTGGA |
| | | TGGGTTGGATTAATCCCAATACGGGCGAGCCCACTTTCGAC |
| | | GACGATTTTAAGGGCAGATTCGCATTCTCTCTGGACACGTC |
| | | AGTAAGCACCGCGTATTTGCAAATTAGCTCCCTTAAAGCG |
| | | GACGACACGGCGGTATATTTTTGCTCTAGAAGTCGCGGCA |
| | | AAAACGAAGCCTGGTTTGCTTACTGGGGCCAGGGCACCTT |
| | | GGTCACGGTCTCCTCT |
| 72 | 537 | GACATACAGCTTACGCAATCACCTTTGTCCCTTCCAGTGAC |
| | | GCTTGGCCAACCTGCCTCCATAAGCTGCAGATCCTCCCAGT |
| | | CACTGGTACATAGAAATGGCAATACCTATTTGCACTGGTTC |
| | | CAGCAGCGGCCCGGCCAATCCCCTCGACTTCTCATATACAC |
| | | CGTGTCAAACCGCTTTTCCGGCGTGCCGGACCGATTCAGTG |
| | | GCTCTGGCTCTGGCACGGACTTTACTCTCAAAATCTCTAGA |
| | | GTCGAGGCGGAGGACGTTGGCGTTTATTTTTGTTCACAGTC |
| | | CAGCCATGTCCCTCCGACTTTCGGGGGGGGAACAAGACTG |
| | | GAGATTAAGGGAGGAGGTGGCTCAGGTGGTGGTGGATCAG |
| | | GGGGAGGTGGATCACAAGTTCAACTCCAACAGTCTGGAAG |
| | | TGAGTTGAAGAAACCCGGAGCTTCAGTCAAGGTCTCCTGT |
| | | AAAGCATCCGGTTACACGTTCACCAATTATGGAGTTAATTG |
| | | GATCAAGCAGGCGCCGGGCCAGGGCCTTCAGTGGATGGGC |
| | | TGGATCAACCCAAATACAGGTGAACCCACGTTCGATGATG |
| | | ATTTTAAGGGTCGATTTGCATTTTCCTTGGATACATCTGTGT |
| | | CTACGGCATATTTGCAAATAAGCAGTCTCAAGGCTGACGA |
| | | TACTGCCGTGTACTTCTGCTCCAGGTCAAGAGGAAAGAAC |
| | | GAGGCGTGGTTCGCCTACTGGGGGCAAGGAACCTTGGTAA |
| | | CGGTTAGTAGT |
| 73 | 5313 | GATATACAGTTGACCCAAAGTCCCCTGTCTTTGCCCGTCAC |
| | | TCTCGGCCAGCCCGCATCCATTTCTTGTCGGTCCTCTCAAA |
| | | GTCTGGTCCACAGGAATGGTAATACCTACTTGCACTGGTTC |
| | | CAGCAACGGCCGGGCCAAAGTCCGCGGCTGCTGATATACA |
| | | CAGTGAGCAATAGGTTTTCAGGCGTACCGGACAGGTTCTCT |
| | | GGTTCCGGCTCTGGTACTGATTTCACGCTGAAAATCTCACG |
| | | AGTGGAAGCTGAGGATGTTGGGGTCTATTTCTGCAGTCAGT |
| | | CCTCCCATGTCCCACCGACATTTGGAGCTGGGACAAGACTC |
| | | GAGATCAAAGGCGGCGGGGGGAGTGGAGGGGGTGGGAGT |
| | | GGTGGAGGAGGTAGCCAAGTCCAGCTTCAGCAAAGTGGAT |
| | | CTGAATTGAAGAAACCGGGAGCCTCTGTCAAGGTGAGCTG |
| | | CAAAGCTAGCGGCTATACCTTTACAAATTACGGAGTCAAC |
| | | TGGATAAAGCAAGCCCCTGGTCAGGGGCTCCAATGGATGG |
| | | GCTGGATCAACCCTAATACCGGTGAACCCACGTGGGACGA |
| | | TGATTTCAAGGGAAGATTCGCATTTTCCCTCGACACGTCAG |
| | | TCAGTACGGCCTACCTCCAAATAAGCTCCCTCAAAGCTGAC |
| | | GATACGGCGGTCTATTTTTGTTCCAGATCCCGAGGGAAGA |
| | | ACGAGGCATGGTTTGCTTACTGGGGTCAAGGCACTTTGGTT |
| | | ACCGTCAGTAGC |
| 92 | CAR | ACAACAACTCCGGCTCCACGGCCTCCTACCCCGGCCCCCAC |
| | backbone | AATCGCTTCACAACCACTGTCACTCAGGCCTGAAGCATGTC |
| | | GGCCCGCCGCTGGCGGTGCTGTGCACACACGAGGCTTGGA |
| | | CTTCGCCTGCGACATTTACATATGGGCACCTTTGGCCGGCA |
| | | CCTGCGGCGTCCTCCTTCTGAGCCTCGTCATAACGCTGTAT |
| | | TGCAAGCGAGGTCGGAAGAAGTTGCTCTATATATTCAAGC |
| | | AACCGTTCATGCGACCGGTACAGACGACCCAGGAAGAGGA |
| | | TGGCTGTTCATGTCGGTTCCCAGAGGAAGAAGAGGGTGGC |
| | | TGCGAGCTTAGGGTCAAATTTTCCAGAAGCGCTGATGCGC |
| | | CTGCTTACCAGCAAGGACAGAACCAACTTTATAACGAACT |
| | | GAATCTCGGACGCCGGGAAGAATATGATGTGTTGGATAAA |
| | | CGGAGGGGCCGCGACCCGGAGATGGGAGGCAAGCCAAGA |
| | | CGCAAAAACCCACAGGAGGGTCTTTACAATGAACTCCAGA |
| | | AAGACAAAATGGCCGAGGCTTATAGTGAGATAGGCATGAA |
| | | GGGCGAGAGAAGACGAGGTAAGGGACACGACGGACTGTA |
| | | TCAAGGGCTGTCCACAGCTACCAAGGACACCTACGACGCC |
| | | CTCCACATGCAGGCTCTCCCGCCCCGATAG |

### REFERENCES

Armitage J O, Longo D L. Mantle-Cell Lymphoma. N Engl J Med. 2022; 386(26):2495-506.
Navarro A, Bei S, Jares P, Campo E. Molecular Pathogenesis of Mantle Cell Lymphoma. Hematol Oncol Clin North Am. 2020; 34(5):795-807.
Jain P, Wang M. Mantle cell lymphoma: 2019 update on the diagnosis, pathogenesis, prognostication, and management. Am J Hematol. 2019; 94(6):710-25.
Hill H A, Qi X, Jain P, Nomie K, Wang Y, Zhou S, et al. Genetic mutations and features of mantle cell lymphoma: a systematic review and meta-analysis. Blood Adv. 2020; 4(13):2927-38.
Cheah C Y, Chihara D, Romaguera J E, Fowler N H, Seymour J F, Hagemeister F B, et al. Patients with mantle cell lymphoma failing ibrutinib are unlikely to respond to salvage chemotherapy and have poor outcomes. Ann Oncol. 2015; 26(6):1175-9.
Campo E, Rule S. Mantle cell lymphoma: evolving management strategies. Blood. 2015; 125(1):48-55.
Kluin-Nelemans H C, Hoster E, Hermine O, Walewski J, Geisler C H, Trneny M, et al. Treatment of Older Patients With Mantle Cell Lymphoma (MCL): Long-Term Follow-Up of the Randomized European MCL Elderly Trial. J Clin Oncol. 2020; 38(3):248-56.
Wang M L, Rule S, Martin P, Goy A, Auer R, Kahl B S, et al. Targeting BTK with ibrutinib in relapsed or refractory mantle-cell lymphoma. N Engl J Med. 2013; 369(6):507-16.
Tam C S, Anderson M A, Pott C, Agarwal R, Handunnetti S, Hicks R J, et al. Ibrutinib plus Venetoclax for the Treatment of Mantle-Cell Lymphoma. N Engl J Med. 2018; 378(13):1211-23.
Wang M, Munoz J, Goy A, Locke F L, Jacobson C A, Hill B T, et al. KTE-X19 CAR T-Cell Therapy in Relapsed or Refractory Mantle-Cell Lymphoma. N Engl J Med. 2020; 382(14):1331-42.
Wang M, Munoz J, Goy A, Locke F L, Jacobson C A, Hill B T, et al. Three-Year Follow-Up of KTE-X19 in Patients With Relapsed/Refractory Mantle Cell Lymphoma, Including High-Risk Subgroups, in the ZUMA-2 Study. J Clin Oncol. 2023; 41(3):555-67.
Schröder B. The multifaceted roles of the invariant chain CD74—More than just a chaperone. Biochim Biophys Acta. 2016; 1863(6 Pt A):1269-81.
Basha G, Omilusik K, Chavez-Steenbock A, Reinicke A T, Lack N, Choi K B, et al. A CD74-dependent MHC class I endolysosomal cross-presentation pathway. Nat Immunol. 2012; 13(3):237-45.
Starlets D, Gore Y, Binsky I, Haran M, Harpaz N, Shvidel L, et al. Cell-surface CD74 initiates a signaling cascade leading to cell proliferation and survival. Blood. 2006; 107(12):4807-16.
Barrera C A, Beswick E J, Sierra J C, Bland D, Espejo R, Mifflin R, et al. Polarized expression of CD74 by gastric epithelial cells. J Histochem Cytochem. 2005; 53(12):1481-9.
Ong G L, Goldenberg D M, Hansen H J, Mattes M J. Cell surface expression and metabolism of major histocompatibility complex class II invariant chain (CD74) by diverse cell lines.
Immunology. 1999; 98(2):296-302.
Stein R, Mattes M J, Cardillo™, Hansen H J, Chang C H, Burton J, et al. CD74: a new candidate target for the immunotherapy of B-cell neoplasms. Clin Cancer Res. 2007; 13(18 Pt 2):5556s-63s.
Haran M, Mirkin V, Braester A, Harpaz N, Shevetz O, Shtreiter M, et al. A phase I-II clinical trial of the anti-CD74 monoclonal antibody milatuzumab in frail patients with refractory chronic lymphocytic leukaemia: A patient based approach. Br J Haematol. 2018; 182(1):125-8.
Alinari L, Mahoney E, Patton J, Zhang X, Huynh L, Earl C T, et al. FTY720 increases CD74 expression and sensitizes mantle cell lymphoma cells to milatuzumab-mediated cell death. Blood. 2011; 118(26):6893-903.
Alinari L, Yu B, Christian B A, Yan F, Shin J, Lapalombella R, et al. Combination anti-CD74 (milatuzumab) and anti-CD20 (rituximab) monoclonal antibody therapy has in vitro and in vivo activity in mantle cell lymphoma. Blood. 2011; 117(17):4530-41.
Alinari L, Baiocchi R A, Praetorius-Ibba M. FTY720-induced blockage of autophagy enhances anticancer efficacy of milatuzumab in mantle cell lymphoma: is FTY720 the next autophagy-blocking agent in lymphoma treatment? Autophagy. 2012; 8(3):416-7.
Stein R, Qu Z, Cardillo™, Chen S, Rosario A, Horak I D, et al. Antiproliferative activity of a humanized anti-CD74 monoclonal antibody, hLL1, on B-cell malignancies. Blood. 2004; 104(12):3705-11.
Martin P, Furman R R, Rutherford S, Ruan J, Ely S, Greenberg J, et al. Phase I study of the anti-CD74 monoclonal antibody milatuzumab (hLL1) in patients with previously treated B-cell lymphomas. Leuk Lymphoma. 2015; 56(11):3065-70.
Leem J, Deane C M. High-Throughput Antibody Structure Modeling and Design Using A BodyBuilder. Methods Mol Biol. 2019; 1851:367-80.
Ambrosetti F, Jandova Z, Bonvin A M J J. Information-Driven Antibody-Antigen Modelling with HADDOCK. Methods Mol Biol. 2023; 2552:267-82.
Yin S, Ding F, Dokholyan N V. Eris: an automated estimator of protein stability. Nat Methods. 2007; 4(6):466-7.
Geuijen C A, Clijsters-van der Horst M, Cox F, Rood P M, Throsby M, Jongeneelen M A, et al. Affinity ranking of antibodies using flow cytometry: application in antibody phage display-based target discovery. J Immunol Methods. 2005; 302(1-2):68-77.
Smith E L, Staehr M, Masakayan R, Tatake I J, Purdon T J, Wang X, et al. Development and Evaluation of an Optimal Human Single-Chain Variable Fragment-Derived BCMA-Targeted CAR T Cell Vector. Mol Ther. 2018; 26(6):1447-56.
Chan W K, Suwannasaen D, Throm R E, Li Y, Eldridge P W, Houston J, et al. Chimeric antigen receptor-redirected CD45R A-negative T cells have potent antileukemia and pathogen memory response without graft-versus-host activity. Leukemia. 2014.
Verma B, Wesa A. Establishment of Humanized Mice from Peripheral Blood Mononuclear Cells or Cord Blood CD34+ Hematopoietic Stem Cells for Immune-Oncology Studies Evaluating New Therapeutic Agents. Curr Protoc Pharmacol. 2020; 89(1):e77.
Myung Y, Rodrigues C H M, Ascher D B, Pires DEV. mCSM-AB2: guiding rational antibody design using graph-based signatures. Bioinformatics. 2020; 36(5):1453-9.
Al-Mansour M. Treatment Landscape of Relapsed/Refractory Mantle Cell Lymphoma: An Updated Review. Clin Lymphoma Myeloma Leuk. 2022; 22(11):e1019-e31.
Parker K R, Migliorini D, Perkey E, Yost K E, Bhaduri A, Bagga P, et al. Single-Cell Analyses Identify Brain Mural Cells Expressing CD19 as Potential Off-Tumor Targets for CAR-T Immunotherapies. Cell. 2020; 183(1):126-42.e17.
Kaufman J L, Niesvizky R, Stadtmauer E A, Chanan-Khan A, Siegel D, Horne H, et al. Phase I, multicentre, dose-escalation trial of monotherapy with milatuzumab (humanized anti-CD74 monoclonal antibody) in relapsed or refractory multiple myeloma. Br J Haematol. 2013; 163(4):478-86.
Beswick E J, Reyes V E. CD74 in antigen presentation, inflammation, and cancers of the gastrointestinal tract. World J Gastroenterol. 2009; 15(23):2855-61.
Rodriguez-Marquez P, Calleja-Cervantes M E, Serrano G, Oliver-Caldes A, Palacios-Berraquero M L, Martin-Mallo A, et al. CAR density influences antitumoral efficacy of BCMA CAR T cells and correlates with clinical outcome. Sci Adv. 2022; 8(39):eabo0514.
Majzner R G, Rietberg S P, Sotillo E, Dong R, Vachharajani V T, Labanieh L, et al. Tuning the Antigen Density Requirement for CAR T-cell Activity. Cancer Discov. 2020; 10(5):702-23.
Alinari L. Toward autophagy-targeted therapy in lymphoma. Blood. 2017; 129(13):1740-2.

What is claimed is:

1. A chimeric antigen receptor (CAR) polypeptide comprising a CD74 antigen binding domain, a transmembrane domain, an intracellular signaling domain, and a co-stimulatory signaling region.

2. The polypeptide of claim 1, wherein the CD74 antigen binding domain is a single-chain variable fragment (scFv) of an antibody that specifically binds to CD74.

3. The polypeptide of any one of claims 1-2, wherein the co-stimulatory signaling region comprises the cytoplasmic domain of costimulatory molecule 4-1BB.

4. The polypeptide of any one of claims 1-3, wherein the intracellular signaling domain comprises a CD3 zeta (CD36) signaling domain.

5. The polypeptide of any one of claims 1-4, wherein the CAR polypeptide is defined by the formula: 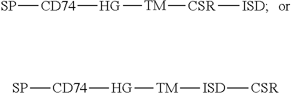
wherein “SP” represents a signal peptide,
wherein “CD74” represents a CD74-binding region,
wherein “HG” represents an optional hinge domain,
wherein “CSR” represents a co-stimulatory signaling region,
wherein “ISD” represents an intracellular signaling domain, and
wherein “-” represents an optional bivalent linker.

6. The polypeptide of any one of claims 1-5, wherein the CAR polypeptide is defined by the formula: 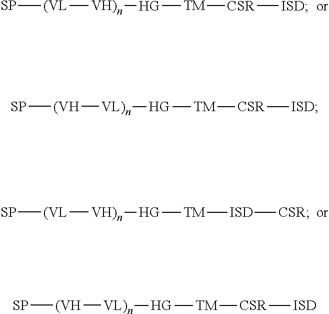
wherein “SP” represents a signal peptide,
wherein “VL” represents a light chain variable region,
wherein “VH” represents a heavy chain variable region,
wherein “n” is ≥1,
wherein “HG” represents an optional hinge domain,
wherein “CSR” represents a co-stimulatory signaling region,
wherein “ISD” represents an intracellular signaling domain, and
wherein “-” represents an optional bivalent linker.

7. The polypeptide of any one of claims 2-6, wherein the scFv of an antibody that specifically binds to CD74 comprises a sequence with at least 60% identity to SEQ ID NOS: 2-7 or a fragment thereof.

8. The polypeptide of claim 6, wherein the scFv of an antibody that specifically binds to CD74 comprises a sequence with at least 60% identity to SEQ ID NO: 2 or a fragment thereof.

9. The polypeptide of any one of claims 2-6, wherein the scFv of an antibody that specifically binds to CD74 comprises a sequence with at least 60% identity to SEQ ID NOS: 8-12 or a fragment thereof.

10. The polypeptide of claim 9, wherein the scFv of an antibody that specifically binds to CD74 comprises a sequence with at least 60% identity to SEQ ID NO: 8 or a fragment thereof.

11. The polypeptide of any one of claims 2-6, wherein the scFv of an antibody that specifically binds to CD74 comprises a sequence with at least 60% identity to SEQ ID NOS: 13-14 or a fragment thereof.

12. The polypeptide of claim 11, wherein the scFv of an antibody that specifically binds to CD74 comprises a sequence with at least 60% identity to SEQ ID NO: 13 or a fragment thereof.

13. The polypeptide of claim 8, wherein the antibody comprises a heavy chain variable region (VH) comprising a sequence with at least 60% identity to SEQ ID NOS: 74, 75, and 76, and a light chain variable region (VL) comprising a sequence with at least 60% identity to SEQ ID NOS: 77, 78, and 79.

14. The polypeptide of claim 10, wherein the antibody comprises a heavy chain variable region (VH) comprising a sequence with at least 60% identity to SEQ ID NOS: 80, 81, and 82, and a light chain variable region (VL) comprising a sequence with at least 60% identity to SEQ ID NOS: 83, 84, and 85.

15. The polypeptide of claim 12, wherein the antibody comprises a heavy chain variable region (VH) comprising a sequence with at least 60% identity to SEQ ID NOS: 86, 87, 88, and a light chain variable region (VL) comprising a sequence with at least 60% identity to SEQ ID NOS: 89, 90, and 91.

16. An isolated nucleic acid sequence encoding the recombinant polypeptide of any one of claims 1-15.

17. A vector comprising the isolated nucleic acid sequence of claim 16.

18. A cell comprising the vector of claim 17.

19. The cell of claim 18, wherein the cell reduces tumor activity when the antigen binding domain of the CAR binds to CD74.

20. A method of treating lymphoma in a subject in need thereof, the method comprising administering to the subject a therapeutically effective amount of the chimeric antigen receptor polypeptide of any one of claims 1-15.

21. The method of claim 20, wherein the lymphoma comprises non-Hodgkin's lymphoma.

22. The method of claim 21, wherein non-Hodgkin's lymphoma comprises mantle cell lymphoma (MCL).

23. The method of claim 22, wherein mantle cell lymphoma comprises Stage I, Stage II, Stage III, or Stage IV mantle cell lymphoma.

24. The method of any one of claims 20-23, further comprising administering a therapeutically effective amount of a CD74 expression modulating agent.

25. The method of any one of claims 20-24, further comprising administering a therapeutically effective amount of an autophagy inhibitor.

26. The method of any one of claims 20-25, further comprising administering a therapeutically effective amount of a histone deacetylase (HDAC) inhibitor.

27. The method of any one of claims 20-26, wherein the chimeric antigen receptor is administered in combination with at least one cancer therapy.

28. The method of claim 27, wherein the additional cancer therapy comprises surgery, chemotherapy, immunotherapy, ionizing radiation, stem cell transplantation (SCT), or a combination thereof.

29. A method of reducing tumor activity in a subject with lymphoma, the method comprising administering to the subject a therapeutically effective amount of the chimeric antigen receptor polypeptide of any one of claims 1-15.

30. The method of claim 29, wherein the lymphoma comprises non-Hodgkin's lymphoma.

31. The method of claim 30, wherein non-Hodgkin's lymphoma comprises mantle cell lymphoma (MCL).

32. The method of any one of claims 29-31, wherein mantle cell lymphoma comprises Stage I, Stage II, Stage III, or Stage IV mantle cell lymphoma.

33. The method of any one of claims 29-32, further comprising administering a therapeutically effective amount of a CD74 expression modulating agent.

34. The method of any one of claims 29-33, further comprising administering a therapeutically effective amount of an autophagy inhibitor.

35. The method of any one of claims 29-34, further comprising administering a therapeutically effective amount of an HDAC inhibitor.

36. The method of any one of claims 29-35, wherein the chimeric antigen receptor is administered in combination with at least one cancer therapy.

37. The method of claim 36, wherein the additional cancer therapy comprises surgery, chemotherapy, immunotherapy, ionizing radiation, stem cell transplantation (SCT), or a combination thereof.


SEQUENCE LISTING (92 sequences)

SEQ ID NO: 1 - protein, 247 aa, synthetic construct
  REGION 1..247: note = Description of sequence: Parent
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGGGGSGGGGSGGGGSQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 2 - protein, 247 aa, synthetic construct
  REGION 1..247: note = Description of sequence: 42105-01
DIQMTQTTSSLSASLGDRVTISCRASQSLVHRNGNTYLNWYQQKPDGTVKLLIYTVSRLHSGVPSRFSGSGSGTDYSLTISNLEQEDIATYFCSQSSHVPPTFGGGTKLEITGGGGSGGGGSGGGGSEVKLQESGPGLVAPSQSLSVTCTVSGYTFTNYGVSWIRQPPRKGLEWLGVINPNTGEPYYNSALKSRLTIIKDNSKSQVFLKMNSLQTDDTAIYYCARSRGKNEAWFAYWGQGTSVTVSS

SEQ ID NO: 3 - protein, 247 aa, synthetic construct
  REGION 1..247: note = Description of sequence: 42105-02
DIVLTQSPLSLPVTLGQPASISCRSSQSLVHRNGNTYLHWFQQRPGQSPRLLIYTVSNRFSGVPDRFSGSGSGTDFTLKISRVEAEDVGVYYCSQSSHVPPTFGQGTRLEIKGGGGSGGGGSGGGGSQVQLVQSGSELKKPGASVKVSCKASGYTFTNYGVNWVRQAPGQGLEWMGWINPNTGEPTFDDDFKGRFVFSLDTSVSTAYLQISSLKADDTAVYFCARSRGKNEAWFAYWGQGTLVTVSS

SEQ ID NO: 4 - protein, 232 aa, synthetic construct
  REGION 1..232: note = Description of sequence: 42105-03
DVVMTQSPLSLPVTLGQPASISCRSSQSLVHRNGNTYLHWFQQRPGQSPRRLIYTVSNRFSGVPDRFSGSGSGTDFTLKISRVEAEDVGVYYCSQSSHVPPTFGQGTRLEIKQVQLVQSGSELKKPGASVKVSCKASGYTFTNYGVNWVRQAPGQGLEWMGWINPNTGEPTFDDDFKGRFVFSLDTSVSTAYLQISSLKAEDTAVYYCARSRGKNEAWFAYWGQGTLVTVSS

SEQ ID NO: 5 - protein, 247 aa, synthetic construct
  REGION 1..247: note = Description of sequence: 42105-04
DIQMTQTTSSLSASLGDRVTISCRASQSLVHRNGNTYLNWYQQKPDGTVKLGIYTVSRGHSGVPSRFSGSGSGTDYSLTISNLEQEDIATYFCSQSSHVPPTFGGGTKLEITGGGGSGGGGSGGGGSEVKLQESGPGLVAPSQSLSVTCTVSGYTFTNYGVSWIRQPPRKGLEWLGVINPNTGEPYYNSALKSRLTIIKDNSKSQVFLKMNSLQTDDTAIYYCARSRGKNEAWFAYWGQGTSVTVSS

SEQ ID NO: 6 - protein, 232 aa, synthetic construct
  REGION 1..232: note = Description of sequence: 42105-05
DIVDTQSPLSLPVTLGQPASISCRSSQSLVHRNGNTYLHWFQQRPGQSPRLGIPTVSNRFSGVPDRFSGSGSGTDFTLKISRVEAEDVGVYYCSQSSHVPPTFGQGTRLEIKQVQLVQSGSELKKPGASVKVSCKASGYTFTNYGVNWVRQAPGQGLEWMGWINPNTGEPTFDDDFKGRFVFSLDTSVSTAGLQISSLKADDTAVYFCARSRGKNEAWFAYWGQGTLVTVSS

SEQ ID NO: 7 - protein, 232 aa, synthetic construct
  REGION 1..232: note = Description of sequence: 42105-06
DVVMTQSPLSLPVTLGQPASISCRSSQSLVHRNGNTYLHWFQQRPGQSPRRLPYTVSNRFSGVPDRFSGSGSGTDFTLKISRVEAEDVGVYYCSQSSHVPPTFGQGTRLEIKQVQLVQSGSELKKPGASVKVSCKASGYTFTNYGVNWVRQAPGQGLEWMGWINPNTGEPTFDDDFKGRFVFSLDTSVSTAGLQISSLKAEDTAVYYCARSRGKNEAWFAYWGQGTLVTVSS

SEQ ID NO: 8 - protein, 247 aa, synthetic construct
  REGION 1..247: note = Description of sequence: 5311-01
DIQMTQTTSSLSASLGDRVTISCRASQSLVHRNGNTYLNWYQQKPDGTVKLLIYTVSRLHSGVPSRFSGSGSGTDYSLTISNLEQEDIATYFCSQSSHVPPTFGGGTKLEITGGGGSGGGGSGGGGSQVQLQQSGSELKKPGASVKVSCKASGYTFTNYGVNWIKQAPGQGLQWMGWINPNTGEPTYDDDFKGRFAFSLDTSVSTAYLQISSLKADDTAVYFCSRSRGKNEAWFAYWGQGTLVTVSS

SEQ ID NO: 9 - protein, 247 aa, synthetic construct
  REGION 1..247: note = Description of sequence: 5311-02
DIVLTQSPLSLPVTLGQPASISCRSSQSLVHRNGNTYLHWFQQRPGQSPRLLIYTVSNRFSGVPDRFSGSGSGTDFTLKISRVEAEDVGVYYCSQSSHVPPTFGQGTRLEIKGGGGSGGGGSGGGGSQVQLVQSGSELKKPGASVKVSCKASGYTFTNYGVNWVRQAPGQGLEWMGWINPNTGEPTYDDDFKGRFVFSLDTSVSTAYLQISSLKADDTAVYFCARSRGKNEAWFAYWGQGTLVTVSS

SEQ ID NO: 10 - protein, 247 aa, synthetic construct
  REGION 1..247: note = Description of sequence: 5311-03
DVVMTQSPLSLPVTLGQPASISCRSSQSLVHRNGNTYLHWFQQRPGQSPRRLIYTVSNRFSGVPDRFSGSGSGTDFTLKISRVEAEDVGVYYCSQSSHVPPTFGQGTRLEIKGGGGSGGGGSGGGGSQVQLVQSGSELKKPGASVKVSCKASGYTFTNYGVNWVRQAPGQGLEWMGWINPNTGEPTYDDDFKGRFVFSLDTSVSTAYLQISSLKAEDTAVYYCARSRGKNEAWFAYWGQGTLVTVSS

SEQ ID NO: 11 - protein, 247 aa, synthetic construct
  REGION 1..247: note = Description of sequence: 5311-04
DIVDTQSPLSLPVTLGQPASISCRSSQSLVHRNGNTYLHWFQQRPGQSPRLGIPTVSNRFSGVPDRFSGSGSGTDFTLKISRVEAEDVGVYYCSQSSHVPPTFGQGTRLEIKGGGGSGGGGSGGGGSQVQLVQSGSELKKPGASVKVSCKASGYTFTNYGVNWVRQAPGQGLEWMGWINPNTGEPTYDDDFKGRFVFSLDTSVSTAGLQISSLKADDTAVYFCARSRGKNEAWFAYWGQGTLVTVSS

SEQ ID NO: 12 - protein, 247 aa, synthetic construct
  REGION 1..247: note = Description of sequence: 5311-05
DVVMTQSPLSLPVTLGQPASISCRSSQSLVHRNGNTYLHWFQQRPGQSPRRLPYTVSNRFSGVPDRFSGSGSGTDFTLKISRVEAEDVGVYYCSQSSHVPPTFGQGTRLEIKGGGGSGGGGSGGGGSQVQLVQSGSELKKPGASVKVSCKASGYTFTNYGVNWVRQAPGQGLEWMGWINPNTGEPTYDDDFKGRFVFSLDTSVSTAGLQISSLKAEDTAVYYCARSRGKNEAWFAYWGQGTLVTVSS

SEQ ID NO: 13 - protein, 245 aa, synthetic construct
  REGION 1..245: note = Description of sequence: 543
DIQMTQSPSSLSASVGDRVTITCRASQGISSWLAWYQQKPEKAPKSLIYAASSLQSGVPSRFSGSGSGTDFTLTISSLQPEDFATYYCQQYNSYPLTFGGGTKVEIKGGGGSGGGGSGGGGSQVQLVESGGGVVQPGRSLRLSCAASGFTFSSYGMHWVRQAPDKGLEWVAVIWYDGSNKYYADSVKGRFTISRDNSKNTLYLQMNSLRAEDTAVYYCARGGTLVRGAMYGTDVWGQGTTVTVSS

SEQ ID NO: 14 - protein, 245 aa, synthetic construct
  REGION 1..245: note = Description of sequence: 543-W
DIQMTQSPSSLSASVGDRVTITCRASQGISSWLAWYQQKPGKAPKLLIYAASSLQSGVPSRFSGSGSGTDFTLTISSLQPEDFATYYCQQYNSYPLTFGGGTKVEIKGGGGSGGGGSGGGGSQVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARGGTLVRGAMYGTDVWGQGTTVTVSS

SEQ ID NO: 15 - protein, 246 aa, synthetic construct
  REGION 1..246: note = Description of sequence: 553
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGGGGSGGGGSGGGGSQVQLQESGGGSVQAGGSLRLSCVASGLVYSLKAYDWGWARQAPGKECELVGRIMPNGATVYIDSVKGRFIISRENSNMTVYLQMNSLKFEDTAVYYCARVGDPSGGWCKGQGTQVTVSS

SEQ ID NO: 16 - protein, 246 aa, synthetic construct
  REGION 1..246: note = Description of sequence: 553-W
DIVDTQSPLSLPVTLGQPASISCRSSQSLVHRNGNTYLHWFQQRPGQSPRLGIPTVSNRFSGVPDRFSGSGSGTDFTLKISRVEAEDVGVYYCSQSSHVPPTFGQGTRLEIKGGGGSGGGGSGGGGSQVQLVESGGGLVQPGGSLRLSCVASGLTFSLKAYDWGWIRQAPGKALEWVGRIMPNGATVYIDSVKGRFTISRDNSKNTVYLQMNSLKAEDTAVYYCARVGDPSGGWCWGQGTLVTVSS

SEQ ID NO: 17 - protein, 244 aa, synthetic construct
  REGION 1..244: note = Description of sequence: 563
DIQMTQSPSSVSASVGDRVTITCRASQGISSWVAWYQQKPGKAPKLLIYGSSRLQSGVPSRFSGSGSGTDFTLTISSLQPEDFATYYCQQYNTYPLTFGGGTKVEIKGGGGSGGGGSGGGGSQVQLVESGGGVVQPGRSLRLSCAASGFNFSDYGMHWVRQAPDKGLEWVAVIYDGSISYYADSVKGRFTISRDNSKNTLYLQMNSLRAEDTAVYYCARGGTVEHGAVYGTDVWGQGATVTVSS

SEQ ID NO: 18 - protein, 244 aa, synthetic construct
  REGION 1..244: note = Description of sequence: 563-W
DIQMTQSPSSVSASVGDRVTITCRASQGISSWVAWYQQKPGKAPKLLIYGSSRLQSGVPSRFSGSGSGTDFTLTISSLQPEDFATYYCQQYNTYPLTFGGGTKVEIKGGGGSGGGGSGGGGSQVQLVESGGGVVQPGRSLRLSCAASGFTFSDYGMHWVRQAPGKGLEWVAVIDDGSISYYADSVKGRFTISRDNSKNTLPLQMNSLRAEDTAVYYCARGGTVEHGAVYGTDVWGQGTTVTVSS

SEQ ID NO: 19 - protein, 247 aa, synthetic construct
  REGION 1..247: note = Description of sequence: 5321
DIQMTQTTSSLSASLGDRVTISCRASQSLVHRNGNTYLNWYQQKPDGTVKLLIYTVSRLHSGVPSRFSGSGSGTDYSLTISNLEQEDIATYFCSQSSHVPPTFGGGTKLEITGGGGSGGGGSGGGGSEVKLQESGPGLVAPSQSLSVTCTVSGYTFTNYGVSWIRQPPRKGLEWLGVINPNTGEPYYNSALKSRLTIIKDNSKSQVFLKMNSLQTDDTAIYYCSRCRGKNEAWFAYWGQGTSVTVSS

SEQ ID NO: 20 - protein, 247 aa, synthetic construct
  REGION 1..247: note = Description of sequence: 534
DIQMTQTTSSLSASLGDRVTISCRASQSLVHRNGNTYLNWYQQKPDGTVKLLIYTVSRLHSGVPSRFSGSGSGTDYSLTISNLEQEDIATYFCSQSSHVPPTFGGGTKLEITGGGGSGGGGSGGGGSQVQLQQSGSELKKPGASVKVSCKASGYTFTNYNVNWIKQAPGQGLQWMGWINPNTGEPTADDDFKGRFAFSLDTSVSTAYLQISSLKADDTAVYFCSRSRGKNEAWFAYWGQGTLVTVSS

SEQ ID NO: 21 - protein, 247 aa, synthetic construct
  REGION 1..247: note = Description of sequence: 532
DIQMTQTTSSLSASLGDRVTISCRASQSLVHRNGNTYLNWYQQKPDGTVKLLIYTVSRLHSGVPSRFSGSGSGTDYSLTISNLEQEDIATYFCSQSSHVPPTFGGGTKLEITGGGGSGGGGSGGGGSQVQLQQSGSELKKPGASVKVSCKASGYTFTNYGMNWIKQAPGQGLQWMGWINPNTGEPTADDDFKGRFAFSLDTSVSTAYLQISSLKADDTAVYFCSRSRGKNEAWFAYWGQGTLVTVSS

SEQ ID NO: 22 - protein, 247 aa, synthetic construct
  REGION 1..247: note = Description of sequence: 5310
DIQMTQTTSSLSASLGDRVTISCRASQSLVHRNGNTYLNWYQQKPDGTVKLLIYTVSRLHSGVPSRFSGSGSGTDYSLTISNLEQEDIATYFCSQSSHVPPTFGGGTKLEITGGGGSGGGGSGGGGSQVQLQQSGSELKKPGASVKVSCKASGYTFTNYGVNWIKQAPGQGLQWMGWINPNTGEPTFDDDFWGRFAFSLDTSVSTAYLQISSLKADDTAVYFCSRSRGKNEAWFAYWGQGTLVTVSS

SEQ ID NO: 23 - protein, 247 aa, synthetic construct
  REGION 1..247: note = Description of sequence: 5319
DIQMTQTTSSLSASLGDRVTISCRASQSLVHRNGNTYLNWYQQKPDGTVKLLIYTVSRLHSGVPSRFSGSGSGTDYSLTISNLEQEDIATYFCSQSSHVPPTFGGGTKLEITGGGGSGGGGSGGGGSQVQLQQSGSELKKPGASVKVSCKASGYTFTNYGVNWIKQAPGQGLQWMGWINPNTGEPTFDDDFWGRFAFSLDTSVSTAYLQISSLKADDTAVYFCSRSREKNEAWFAYWGQGTLVTVSS

SEQ ID NO: 24 - protein, 247 aa, synthetic construct
  REGION 1..247: note = Description of sequence: 5315
DIQMTQTTSSLSASLGDRVTISCRASQSLVHRNGNTYLNWYQQKPDGTVKLLIYTVSRLHSGVPSRFSGSGSGTDYSLTISNLEQEDIATYFCSQSSHVPPTFGGGTKLEITGGGGSGGGGSGGGGSQVQLQQSGSELKKPGASVKVSCKASGYTFTNYGVNWIKQAPGQGLQWMGWINPNTGEPTFDDDFWGRFAFSLDTSVSTAYLQISSLKADDTAVYFCCRCREKNEAWFAYWGQGTLVTVSS

SEQ ID NO: 25 - protein, 247 aa, synthetic construct
  REGION 1..247: note = Description of sequence: 571
DIQLTQSPLSLPVTLGQPASISCRSSQSEVHRNGNTYLHWFQQRPGQSPRLLIYTVSNRFSGVPDRFSGSGSGTDFTLKISRVEAEDVGVYFCSQSSHVPPTFGAGTRLEIKGGGGSGGGGSGGGGSQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 26 - protein, 247 aa, synthetic construct
  REGION 1..247: note = Description of sequence: 573
DIQLTQSPLSLPVTLGQPASISCRSSQSLWHRNGNTYLHWFQQRPGQSPRLLIYTVSNRFSGVPDRFSGSGSGTDFTLKISRVEAEDVGVYFCSQSSHVPPTFGAGTRLEIKGGGGSGGGGSGGGGSQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 27 - protein, 247 aa, synthetic construct
  REGION 1..247: note = Description of sequence: 576
DIQLTQSPLSLPVTLGQPASISCRSSQSLVHRNGNTYCHWFQQRPGQSPRLLIYTVSNRFSGVPDRFSGSGSGTDFTLKISRVEAEDVGVYFCSQSSHVPPTFGAGTRLEIKGGGGSGGGGSGGGGSQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 28 - protein, 247 aa, synthetic construct
  REGION 1..247: note = Description of sequence: 578
DIQLTQSPLSLPVTLGQPASISCRSSQSLVHRNGNTYLHWFQQRPGQSPRLLIYEVSNRFSGVPDRFSGSGSGTDFTLKISRVEAEDVGVYFCSQSSHVPPTFGAGTRLEIKGGGGSGGGGSGGGGSQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 29 - protein, 247 aa, synthetic construct
  REGION 1..247: note = Description of sequence: 579
DIQLTQSPLSLPVTLGQPASISCRSSQSLVHRNGNTYLHWFQQRPGQSPRLLIYTESNRFSGVPDRFSGSGSGTDFTLKISRVEAEDVGVYFCSQSSHVPPTFGAGTRLEIKGGGGSGGGGSGGGGSQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 30 - protein, 247 aa, synthetic construct
  REGION 1..247: note = Description of sequence: 5711
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSCSSHVPPTFGAGTRLEIKGGGGSGGGGSGGGGSQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 31 - protein, 247 aa, synthetic construct
  REGION 1..247: note = Description of sequence: 5718
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHWPPTFGAGTRLEIKGGGGSGGGGSGGGGSQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 32 - protein, 247 aa, synthetic construct
  REGION 1..247: note = Description of sequence: 5721
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPCFGAGTRLEIKGGGGSGGGGSGGGGSQVQLQQSGSELKKPGASVKVSCKASGYTFTNYGVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 33 - protein, 247 aa, synthetic construct
  REGION 1..247: note = Description of sequence: 532
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGGGGSGGGGSGGGGSQVQLQQSGSELKKPGASVKVSCKASGYTFTNYGMNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 34 - protein, 247 aa, synthetic construct
  REGION 1..247: note = Description of sequence: 536
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGGGGSGGGGSGGGGSQVQLQQSGSELKKPGASVKVSCKASGYTFTNYWVNWIKQAPGQGLQWMGWINPNTGEPTFDDDFKGRFAFSLDTSVSTAYLQISSLKADDTAVYFCSRSRGKNEAWFAYWGQGTLVTVSS

SEQ ID NO: 35 - protein, 247 aa, synthetic construct; identical to SEQ ID NO: 1
  REGION 1..247: note = Description of sequence: 537

SEQ ID NO: 36 - protein, 247 aa, synthetic construct
  REGION 1..247: note = Description of sequence: 5313
DIQLTQSPLSLPVTLGQPASISCRSSQSLVHRNGNTYLHWFQQRPGQSPRLLIYTVSNRFSGVPDRFSGSGSGTDFTLKISRVEAEDVGVYFCSQSSHVPPTFGAGTRLEIKGGGGSGGGGSGGGGSQVQLQQSGSELKKPGASVKVSCKASGYTFTNYGVNWIKQAPGQGLQWMGWINPNTGEPTWDDDFKGRFAFSLDTSVSTAYLQISSLKADDTAVYFCSRSRGKNEAWFAYWGQGTLVTVSS

SEQ ID NO: 37 - protein, 223 aa, synthetic construct
  REGION 1..223: note = Description of sequence: CAR backbone
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - DNA, 741 bp, synthetic construct
  misc_feature 1..741: note = Description of sequence: Parent
  source 1..741: mol_type = other DNA
gatattcagttgactcaatctcccctctctctccccgttaccctgggccaacctgcctccatatcatgtcggtcctcacaatcacttgtacaccgcaatgggaacacttaccttcactggttccagcaacggccaggtcaatctccacggttgctgatatatactgtaagcaatcggtttagtggtgtccctgaccgcttcagtggttcagggtcaggaactgactttaccctgaaaattagtcgggtggaggctgaggatgtaggcgtctacttttgcagccagtcctcacacgttccgccaacgtttggtgcgggtactagacttgaaataaagggtggcggcggaagtggcggcgggggttctggtggaggcggttcccaggtgcaacttcaacaaagcggtagtgaactgaagaagccgggggcgagcgtcaaggtatcctgtaaggcttcaggctatacgttcaccaactatggcgtcaactggatcaagcaggcaccgggccagggattgcaatggatgggctggataaatccaaacacgggagagcctacctttgatgacgattttaaaggacggtttgcatttagtcttgacacgagcgtgtcaacggcgtacttgcagatatcatctctcaaggccgacgatacggcagtgtatttttgttctcggtcaaggggcaaaaacgaggcatggtttgcgtattggggtcaggggacgctggtcacagtgtcctct

SEQ ID NO: 39 - DNA, 741 bp, synthetic construct
  misc_feature 1..741: note = Description of sequence: 42105-01
  source 1..741: mol_type = other DNA
gatatacaaatgacccagaccacttcaagtttgtcagccagcttgggggacagggtcactatatcatgccgagcgtctcagtctttggtccatcgcaatgggaatacctatcttaactggtatcaacagaaacccgatggcacggtcaaactgttgatatacaccgtttcccgacttcatagcggggtgcctagtcgattttccggatccggctccggtacggattattctttgacgatctccaacctggaacaggaggatattgcaacgtatttttgctcacagagttcacatgtacccccgacgttcggtggtggtacgaaactcgagatcactggcgggggcggttcaggaggcggtggatctggtggaggaggatccgaggtaaaactgcaagaatctggaccaggtctggtggctccttcacagagcctttccgtcacatgcacagtctctggttacacgttcaccaattatggagtttcatggattaggcaaccgccaagaaaaggtctcgagtggctcggggtgattaatcccaacacgggtgagccttattataattcagccttgaagtctcgccttacgattataaaagacaactcaaagtcacaagtattcctgaaaatgaactccctgcagaccgatgatacggcgatttattactgcgccagatcacgaggaaaaaatgaagcttggttcgcgtactggggccaagggacaagtgtgacagtaagcagt

SEQ ID NO: 40 - DNA, 741 bp, synthetic construct
  misc_feature 1..741: note = Description of sequence: 42105-02
  source 1..741: mol_type = other DNA
gacatcgtcttgacacaatcccccctttcccttcctgtcacgcttggccagcccgcctctatcagttgtagatccagccaaagtctggtacaccggaatgggaatacatatctgcattggttccagcaacggccaggtcagagtcctaggttgcttatttatacggtctccaaccggttcagtggagtacccgaccggttttctggctcaggcagcggaaccgactttacattgaaaatttcacgcgtcgaagccgaggatgttggtgtgtattattgttcccaaagctctcacgtccccccgacttttggtcagggaactaggcttgaaatcaaaggtggcgggggaagtggtggcgggggatctgggggtgggggatctcaagtgcagctcgtgcagtccgggagcgaattgaaaaaaccaggggcaagcgtgaaggtttcttgtaaagcctctggatatactttcaccaattacggggtaaattgggttcggcaagcaccaggacaaggactggagtggatgggttggattaatccaaatacgggcgagcccacgtttgacgatgactttaaggggcgatttgtattcagcttggacacaagcgtgtccacagcgtaccttcaaatatcatctcttaaggcggacgatactgcagtctacttctgtgctcggagtcggggaaaaaacgaggcatggtttgcttattggggtcagggcacgcttgttactgtctcttct

SEQ ID NO: 41 - DNA, 696 bp, synthetic construct
  misc_feature 1..696: note = Description of sequence: 42105-03
  source 1..696: mol_type = other DNA
gatgttgttatgacacagtccccattgagcctcccagtgactttgggacagcctgcgagtatatcctgtaggagctcccaatcactcgttcaccgaaatggtaacacgtacctgcactggtttcagcaaaggccaggtcaatcacctcgaagactcatctacacggtcagcaaccgcttctctggggtgccggacaggttttccgggtcaggctccggtacagactttacactgaaaatctccagagtggaggcagaagatgtaggcgtttattactgctcccaaagtagtcatgtccctcctactttcgggcaagggactagacttgagataaaacaagtccaactcgttcaatccggatcagaactgaaaaagccgggtgcatccgtcaaagtcagttgcaaagcgagtggatacacgttcactaattatggtgttaattgggttcggcaggcgccaggtcaagggttggagtggatgggttggattaaccctaataccggcgaaccgacattcgatgacgacttcaaaggccgattcgtcttcagtcttgacacttcagtaagtaccgcttatcttcagatcagcagtttgaaagctgaagacaccgcggtgtactactgcgcacggagtaggggaaagaatgaagcctggttcgcatattggggacaggggacgctggtgaccgtcagcagc

SEQ ID NO: 42 - DNA, 741 bp, synthetic construct
  misc_feature 1..741: note = Description of sequence: 42105-04
  source 1..741: mol_type = other DNA
gacatacagatgactcaaacgacgagctcactgtccgcgtcacttggcgacagagtgacgatcagctgccgcgcgagccaatctttggtacaccggaacggtaacacctatcttaactggtaccaacaaaagcccgatgggacagtcaaactgggaatctacactgtctctcggggccatagcggagtcccctctcgctttagcggatcaggatctggtactgactattcccttactatctctaacctggaacaagaagacatagccacctatttctgttcacaaagctctcatgtacctccgactttcgggggagggactaaacttgagatcactggtggtggcgggagcggaggagggggaagtggtggcggtggatcagaggtgaagcttcaagaatccggaccgggcctcgtcgccccttcacaaagcctgagtgtgacttgtacggtgagcggatacacgtttactaactatggcgtatcttggataagacagccgcctcgcaagggattggagtggctcggggtaataaaccccaacaccggagagccctattacaattctgctctcaagtcaagactcacgattatcaaagataactcaaagtctcaggtttttctgaaaatgaacagtcttcaaacggatgacaccgcaatctactactgtgctcgatcccgaggcaaaaatgaggcatggtttgcgtactggggtcaagggacctccgtaacagtctcttcc

SEQ ID NO: 43 - DNA, 696 bp, synthetic construct
  misc_feature 1..696: note = Description of sequence: 42105-05
  source 1..696: mol_type = other DNA
gacattgtagatacgcaatctcccctttccttgcccgtgacgttgggccagccggcctccataagctgccgatcaagtcagtctctcgtccacaggaatgggaacacttacctccattggttccagcaacgacccgggcaatcaccgcgactgggcatacccactgtaagcaatagatttagcggcgtacctgacagattctctggtagtggcagtggcacagactttaccctcaaaatcagtcgcgttgaagctgaggacgttggcgtgtactactgctcacaaagctcccacgtaccgcccacatttggtcaaggaacgcgccttgaaataaaacaagtacagctcgtccagagcggctcagaactcaaaaaaccgggggcttcagtgaaagtatcttgcaaagcaagtgggtatacgttcacgaattacggcgttaactgggtaaggcaagcgcctgggcagggccttgagtggatgggttggataaatcctaatacaggtgagccaacttttgatgacgactttaagggacggtttgttttctcactcgacacttctgtaagtacggcagggctccaaatctctagtcttaaagctgacgacacagctgtttatttctgcgctagaagtcgcgggaagaacgaggcgtggtttgcgtattggggccagggtacgctcgtaaccgttagctct

SEQ ID NO: 44 - DNA, 696 bp, synthetic construct
  misc_feature 1..696: note = Description of sequence: 42105-06
  source 1..696: mol_type = other DNA
gacgttgttatgactcaaagccctctgtcacttcctgtcactcttgggcaacccgccagtatctcatgtcgaagctctcagagtctcgtacaccggaatgggaatacatatcttcactggttccaacagcgcccaggacaatctccccgacgccttccatataccgtcagcaatagattttcaggagtacctgaccggttctcaggttctggttcaggtaccgattttacacttaagataagccgggtagaagccgaagatgtgggggtctactattgtagccagagttcacacgttcccccgacgtttgggcaaggcacgcgcctcgagatcaagcaggtccaactggtccagagcgggtcagagctgaagaagcccggtgcgtcagttaaggtctcctgcaaagcctcaggatatacctttaccaattacggagtgaactgggtgagacaagctccgggtcaggggttggagtggatgggatggataaatcccaatacgggcgaaccaacattcgatgacgactttaaaggcagattcgtattctcattggacacgtctgtcagtacagcaggactgcaaatatcatccctgaaagcagaggacacggcagtttactattgtgcgcgaagccggggaaagaatgaagcttggtttgcttactggggacagggcaccctggtaaccgtctcctct

SEQ ID NO: 45 - DNA, 741 bp, synthetic construct
  misc_feature 1..741: note = Description of sequence: 5311-01
  source 1..741: mol_type = other DNA
gacatccaaatgactcagactacctcctcactctcagcgagcctcggcgatcgagttacgatctcttgtagagcaagccaaagtttggttcatagaaacggaaacacatatctgaattggtatcagcaaaaaccggacgggactgtgaaacttctgatctacacagtgtctcgcttgcatagtggggtcccaagcaggttctctggtagtggctcaggtactgactattcactcaccatctccaaccttgaacaagaagatatcgccacctatttctgctcccagagctcacacgtcccacccacctttggcgggggcaccaagctggagattaccgggggaggcggtagtgggggtgggggaagtggaggtggtggctcccaagtccagctgcaacaatctggtagcgagttgaagaaacctggggctagtgttaaagttagttgcaaggcctccggatataccttcacgaattacggggtaaactggatcaagcaagctcccggacaaggactgcagtggatgggttggattaaccccaacacaggagaaccgacgtacgatgacgatttcaaagggcggttcgccttctccttggatacatctgtttccaccgcctatctccaaatttcttctctcaaagccgacgacacagctgtgtatttttgttcacgctcacgcggtaagaacgaagcttggttcgcgtactggggccaagggacccttgtaacggtgtcaagc

SEQ ID NO: 46 - DNA, 741 bp, synthetic construct
  misc_feature 1..741: note = Description of sequence: 5311-02
  source 1..741: mol_type = other DNA
gatatcgtactgacgcaatctccgctgagcctgccagttactttgggccagcctgcaagcatctcttgtaggtcatcacaaagtcttgtccaccgaaatggcaacacatatctgcattggtttcagcaaaggccgggtcagagtccccgcttgctgatatacactgtgtccaatcggttctcaggggtgccagacaggttttctggaagcggaagtgggactgactttacgcttaaaatatcccgagtagaagcggaagatgtcggagtgtactactgttcccagagctcccatgtgcctccgactttcggtcaggggacaaggcttgaaatcaagggcgggggcggttcaggcggtggaggatctggcggaggtggatcccaggtccagttggtgcaaagcggttctgaactgaaaaagcctggagcatctgtaaaagtgagctgcaaagctagtggctatacgtttactaactatggtgtcaactgggttaggcaggcgccggggcagggattggaatggatgggctggattaatcctaacaccggtgaaccgacttacgatgacgacttcaaaggccgctttgtgttcagcctcgatacgagcgtcagtaccgcatacttgcaaatatcaagtctcaaagcagacgacacagcagtttatttctgtgctcggtcacggggtaaaaatgaagcgtggtttgcatactggggccaaggaacattggtaaccgttagcagc

SEQ ID NO: 47 - DNA, 741 bp, synthetic construct
  misc_feature 1..741: note = Description of sequence: 5311-03
  source 1..741: mol_type = other DNA
gacgtggtaatgactcagagccctttgtccttgcccgtgactctgggtcaaccggcgtctatcagctgtagatcttcccaaagcttggtccaccgcaatggcaatacataccttcattggtttcagcagcggcctgggcagtctccaaggcgcttgatatataccgtctccaatcggttctccggcgttcccgatagattcagcggttcagggagtggaacggattttacgctgaaaataagtagagtcgaggcagaagatgtcggcgtctactactgctcccaatcatcacatgttcctcctacatttggtcaagggacaagacttgagattaagggcggtggaggctcaggagggggcggaagcggtgggggtggatctcaagtgcagctcgtacagagcggatctgaactcaaaaagcctggggctagcgtaaaagtatcatgtaaagcgagcggatataccttcactaattacggcgtgaactgggttcgccaagcgcccggacaagggctcgaatggatggggtggataaatcccaacacgggcgaacctacctacgatgacgactttaaaggccggtttgtgtttagtctcgacacgagtgtcagcacggcttacctgcagatttcatcactgaaggcggaggacaccgctgtctactactgcgctagaagtagaggaaaaaacgaagcgtggtttgcctattggggtcaaggtacgctcgttacagtcagctct

SEQ ID NO: 48 - DNA, 741 bp, synthetic construct
  misc_feature 1..741: note = Description of sequence: 5311-04
  source 1..741: mol_type = other DNA
gacattgtagatacgcaatcaccattgagcctgcctgtcacacttggtcaaccggcatctattagttgtcggagttcccagtccctcgtccatcgaaacggaaatacgtatttgcattggtttcagcagcgccctggtcagtcaccaagactgggcattcccacagttagtaaccgcttttcaggagtccccgacaggttcagtgggagtggctccggcactgattttacgcttaagataagccgggttgaggccgaagacgtaggcgtctattactgctctcagtcctcacatgttcccccaacattcgggcagggcacccgattggaaattaagggaggtggaggctcaggcggcggaggttctgggggaggcggttcccaagttcagcttgtgcagagtggatcagagctgaagaagccgggggcctctgttaaagtgtcctgtaaagcatcagggtacaccttcactaactatggagtaaactgggtgcgacaggcgcctgggcaaggcttggaatggatgggatggatcaatcctaataccggggaaccaacctacgatgatgactttaaggggcgcttcgtcttcagcttggacacgtcagttagcacggcggggttgcaaatatcatcactcaaggcagatgatacagctgtttatttttgtgctcgaagtagaggcaaaaacgaagcctggttcgcatattggggacaaggtacacttgtgacggtctcctca

SEQ ID NO: 49 - DNA, 741 bp, synthetic construct
  misc_feature 1..741: note = Description of sequence: 5311-05
  source 1..741: mol_type = other DNA
gatgtggtaatgactcaaagtccgctgagcctgcccgtaacgttggggcaacccgcttcaatctcctgtagatcaagccagtcactggtccacagaaatggcaatacatatttgcactggttccaacagcggccaggacagtctcccagacgactcccttatactgtctctaatcggttttctggcgttccggatagatttagtggcagcggcagcgggaccgactttaccctgaagatttcacgagttgaggctgaagatgtaggagtgtattattgcagtcaaagcagtcacgttccaccaactttcggccagggtacgaggctggaaattaagggaggcggtgggtcagggggaggcggatctggaggcggcggaagtcaagtgcagcttgttcagtcagggagcgaactcaagaaacccggagcgtctgttaaagtcagttgtaaggcatccggttataccttcaccaactatggggtaaactgggtaagacaagcgccagggcaaggtctggagtggatgggctggatcaaccctaataccggtgaaccaacttatgatgatgacttcaaagggaggtttgtgttcagtctcgacacgtcagtctccactgctgggctgcaaatctccagtttgaaggcagaagacacagccgtctattattgcgcccgcagccgcggcaagaatgaggcgtggttcgcatactgggggcagggaacgcttgtcactgtgagcagt

SEQ ID NO: 50 - DNA, 735 bp, synthetic construct
  misc_feature 1..735: note = Description of sequence: 543
  source 1..735: mol_type = other DNA
gatatacaaatgacccagtcaccttcaagcttgtcagcttccgtcggcgacagggttacgatcacttgtagagcctctcagggtataagttcttggctggcctggtaccaacaaaaacccgaaaaggccccgaaaagcctgatatacgcggcgagttctctgcagagcggagttccctctaggttttcagggtctgggtcaggaacagatttcacgctcacaatcagttctttgcagccggaggacttcgcgacctattattgccaacaatacaactcctacccgttgacattcggaggagggacgaaagttgaaatcaaaggcgggggagggtctgggggaggcggctctggtgggggtggctctcaagtccaactggttgaatctgggggaggcgtcgttcagccagggcgatccctgagactctcctgcgctgcatctggtttcacgttctcatcatacggaatgcactgggtaaggcaggcgccagacaaggggctggaatgggttgctgttatttggtatgatggctccaacaagtattatgctgattctgttaagggtaggttcaccattagccgagataactcaaagaacactctctatcttcaaatgaatagccttcgcgcagaagacacggcagtttattactgtgcaaggggaggaactcttgtccgaggggcaatgtatggtacggatgtctggggtcaagggaccacggttacagtgtcatct

SEQ ID NO: 51 - DNA, 735 bp, synthetic construct
  misc_feature 1..735: note = Description of sequence: 543-W
  source 1..735: mol_type = other DNA
gatattcaaatgactcagtctccgagctcactgtctgcttcagttggggacagagttactataacgtgccgcgcatcccagggaattagcagttggctggcctggtaccaacaaaagccaggcaaagccccgaagctgttgatatatgcagcttcttctttgcaaagtggggtaccatctcgattctccggctcagggtctggaacagactttaccctgacgattagcagtctccagcctgaagactttgcgacttactattgtcagcaatataattcctatccactgaccttcggcggggggaccaaagtcgaaattaaagggggcggaggctccgggggaggtgggtctgggggaggagggagtcaagttcaactcgtagagtccggaggcggtgtggtacaacctggtaggtccctgcgactctcttgtgcagctagcggttttaccttttcttcttatggtatgcactgggttaggcaagctcccgggaagggcctggaatgggtcgcagtgatttggtacgacggaagtaacaagtactatgctgactcagttaagggccgatttaccatctcacgagataattccaaaaatacactgtacctccaaatgaatagcctccgggcggaggacacggctgtgtattattgtgcgcgaggtggtacgttggtgcgcggagctatgtatggtactgacgtttggggtcagggtacgaccgtcacggtaagctct

SEQ ID NO: 52 - DNA, 738 bp, synthetic construct
  misc_feature 1..738: note = Description of sequence: 553
  source 1..738: mol_type = other DNA
gatatccaactcacgcagagccccctgtcccttccggtgaccctggggcaacccgctagtatttcttgtcgatcctcccaatctcttgttcaccgaaacggtaacacctacctgcactggtttcaacaacggccaggacagagccccaggcttttgatatatacggtctcaaatagattcagtggagtccctgaccgattttccggcagcggctcaggcacggattttacgctgaagataagccgggtcgaagcggaagacgttggtgtttacttttgctctcaatcctctcacgttccaccgaccttcggcgccggcacacgccttgagataaaagggggcggaggaagcggtgggggagggagtggaggaggggggtcacaagtccaactgcaagaatcaggtggtggatccgtccaagctggaggttctcttcgacttagttgcgtggcttctggtctggtgtactcattgaaagcatacgattggggctgggcccgacaagctcctggcaaagaatgtgagctggtgggtcggattatgccgaacggcgctactgtttacattgattccgttaaaggtcggttcatcatttcccgcgaaaattctaatatgactgtataccttcaaatgaatagccttaaattcgaggacacggcagtctattactgcgccagggtaggggacccaagcggaggatggtgtaaaggacaaggtactcaagttacagtaagttct

SEQ ID NO: 53 - DNA, 738 bp, synthetic construct
  misc_feature 1..738: note = Description of sequence: 553-W
  source 1..738: mol_type = other DNA
gacattgttgatactcaatctcctctctctttgccagtcacccttgggcagccagcttctatctcctgtcgctcaagccagtctctcgtgcacaggaatggcaatacgtacctgcattggttccaacaaaggcccggtcagtcaccccgcctgggcattcccaccgtatcaaataggttttctggtgtacccgacaggttttccgggagcggaagcggcactgacttcacactcaaaatatcaagggtcgaagcggaggatgttggtgtttactactgtagtcagtctagtcatgtgccgcccaccttcggccagggcacgcgactggaaatcaagggagggggtggctctggaggcggcggaagcggaggtggtggatcccaggttcaactcgtagagtcaggaggaggactcgtccaacccggtgggagcctccgacttagctgcgtggcgtctggacttactttcagtctgaaggcgtatgattggggatggattcgacaagcccctggtaaggccctcgaatgggtcggacggataatgcccaacggagcgacggtttacatcgactctgtgaaaggaagatttactatctcacgagacaacagcaaaaacacggtttatcttcagatgaattcactcaaggcagaggatactgctgtatactattgtgcgcgcgtgggggacccttcaggcgggtggtgttggggccaaggcacgctcgttactgtatcaagc

SEQ ID NO: 54 - DNA, 732 bp, synthetic construct
  misc_feature 1..732: note = Description of sequence: 563
  source 1..732: mol_type = other DNA
gacatccagatgacccaatccccaagttcagtttcagcttctgttggtgaccgagtcacaattacttgtcgggcgtcacaaggaatcagtagttgggtcgcgtggtatcaacaaaaacccgggaaagcgccgaaactccttatatatggatcctcaaggttgcaatctggagtaccttcccgctttagcggctcaggatccgggaccgatttcacactcaccatttcttcacttcaacctgaggatttcgccacttactattgtcaacagtacaatacttacccattgacttttggcggaggcacaaaagtagagatcaagggcggtggaggttccggaggtggagggtctggcggaggggggtctcaagtacagctcgttgagtcaggtggtggtgtagtccagccaggccggagtttgcgattgagttgcgctgcctctggttttaatttctctgactacgggatgcattgggtccgccaggcccctgacaaaggtctcgagtgggttgcagtgatctatgatggttcaatctcatactatgcggattcagtaaagggacggtttacgatctccagagacaactcaaagaatacgctctatctccagatgaacagcctccgcgcagaagatacagccgtgtattattgtgcaaggggaggaaccgttgaacatggagcggtatatggcactgatgtctggggtcagggggctacagttacggtatcatca

SEQ ID NO: 55 - DNA, 732 bp, synthetic construct
  misc_feature 1..732: note = Description of sequence: 563-W
  source 1..732: mol_type = other DNA
gacatacaaatgactcagagcccctcctctgtgtcagcctcagtaggtgatagggtaaccattacctgtcgagcatcccaaggtatttcatcctgggtcgcatggtatcagcaaaaacccgggaaagcacctaaactcctgatctacggttcttcccggcttcaatccggtgttcctagccggttctcagggagcggatctggaacagactttaccttgacgattagctctctccagcccgaggattttgcaacctactactgccaacaatataatacatacccattgacgtttggaggaggcactaaagtagagattaaagggggagggggttcaggaggaggtggttccggtgggggtggcagtcaagtccagcttgtagagtctggtggcggggtagtacagccaggtcgatctttgcgcttgagctgcgccgcctccggttttactttcagtgactatggaatgcattgggttcggcaagctcccgggaaaggccttgagtgggtggcggtcattgatgacggaagtatttcctactacgcggatagtgttaagggaaggtttaccatatcaagggacaattccaaaaatacacttcctcttcaaatgaattcactccgggctgaggacactgccgtctactattgtgctcgagggggtacagtagagcatggagccgtttacggtactgacgtatggggtcaaggtacaacagtaactgtaagctca

SEQ ID NO: 56 - DNA, 741 bp, synthetic construct
  misc_feature 1..741: note = Description of sequence: 5321
  source 1..741: mol_type = other DNA
gacatccagatgactcaaacgacaagctccttgtctgccagccttggtgatcgggttactatatcctgtcgggccagtcagtcccttgtacatcgcaatggcaatacttatcttaactggtatcagcaaaaacccgatgggaccgttaagcttctcatctatactgtatctcgattgcattcaggggttccttcccgatttagtggaagcggtagtggaacggattatagtctgacaatttccaatcttgaacaagaagacatagcaacttacttttgctcccaatcctcacacgtccccccaacttttggaggaggtaccaagctggagatcacagggggcggagggtcaggtggaggtggaagtggaggtggtggttcagaggtgaagttgcaagaatccggtccgggactcgtggcccctagccaatccttgagtgtcacgtgtacagtctcaggttatacttttacaaattacggggtgagctggatacggcagccacctaggaaaggactcgaatggctcggagtcatcaaccccaacacgggcgaaccgtattataactctgccctcaaaagtcggcttacaataatcaaagacaacagcaaaagccaagtgtttttgaagatgaactccctccagaccgacgatactgcaatttattattgttccagatgtcggggaaagaacgaggcctggttcgcgtactggggtcaaggtacctccgtaaccgtttccagc

SEQ ID NO: 57 - DNA, 741 bp, synthetic construct
  misc_feature 1..741: note = Description of sequence: 534
  source 1..741: mol_type = other DNA
gacatccagatgacgcagacgacgagtagcctgtctgcgtctcttggtgatcgagtcactataagttgccgagcatctcaatccctcgttcatcgcaatgggaacacttacctcaactggtatcaacagaagcctgacggtacggtcaagctgctgatctatacggtgtcaaggctccactctggagttcccagcaggttttccggaagcgggagcggtacagattattctctcactatttctaatctggagcaagaagacatagcaacctatttctgctcacagtcctctcacgtaccccctactttcggcggaggtacaaaactggagattacagggggtggtggttccggaggaggaggtagcgggggaggagggagccaggtacagctccagcagtctggcagtgagcttaaaaaaccaggggcttctgtcaaagtctcctgtaaagcgagcggttataccttcaccaactataatgtaaactggataaaacaggcgcccgggcaagggctccagtggatgggatggatcaatccaaacacgggcgagcctaccgctgatgacgattttaagggccgatttgctttctcattggacacgtccgttagcaccgcatatctccaaatatcaagcctcaaggcggacgacacggctgtttatttttgtagtaggtctagggggaaaaacgaagcgtggtttgcgtattggggccagggtacactggtgactgtctcttca

SEQ ID NO: 58 - DNA, 741 bp, synthetic construct
  misc_feature 1..741: note = Description of sequence: 532
  source 1..741: mol_type = other DNA
gatattcagatgactcaaactacttccagcctctcagcgtctctgggtgaccgagtcacaattagttgcagagcgtctcagtctctggttcaccgcaacgggaacacgtatttgaattggtatcaacaaaaaccggatggcacagtgaaacttctcatctacacggttagcagattgcactcaggcgtcccaagtcgattctccggcagcggtagtggaacagattatagccttacaatctccaatctggaacaagaagacatcgcgacttatttttgttcccagtcttcccacgttccgcctaccttcggcgggggcaccaaactggagattaccgggggaggtggtagtggtggcggtggaagcggagggggcggcagtcaagtgcagttgcaacagtctggttccgaactgaaaaagccgggggcctcagtgaaagtctcttgcaaagcatctggctatactttcactaattatgggatgaactggataaaacaagcccccgggcaaggccttcaatggatggggtggataaatccaaatacaggagagccgacggcggatgatgattttaagggcaggttcgcgttcagcctggacactagcgtatcaacagcatatctccaaataagcagtcttaaggcggacgataccgccgtttatttctgcagccgaagccgcgggaaaaacgaagcctggttcgcgtattgggggcagggcacactcgtgaccgttagtagc

SEQ ID NO: 59 - DNA, 741 bp, synthetic construct
  misc_feature 1..741: note = Description of sequence: 5310
  source 1..741: mol_type = other DNA
gatatacagatgacgcaaactaccagctcattgtcagcgtcccttggtgaccgcgttacaattagttgtagggcctcccaatctctcgtccaccggaacggtaatacctatctgaattggtaccagcagaaaccggatggtaccgttaagctcttgatatatacagtgagcagattgcacagtggcgttccgtctagattcagtggatcaggttccggcaccgattactccctgacaatttctaatttggagcaagaagatatcgcaacgtacttttgtagccaatcctctcatgtcccgcctacgtttggcggcggcactaaactggagataaccgggggcggtggctctggaggcggtggatccggcggagggggcagtcaggttcagttgcaacaatcaggctctgaattgaagaaaccgggtgcctctgtgaaggtgtcctgcaaggcgagcgggtatacatttactaactacggtgtcaattggattaagcaggctccgggccagggattgcagtggatggggtggattaacccaaacacaggcgagccaactttcgacgacgacttttggggaagattcgctttttccctggacactagcgtgtctaccgcttatctccaaatatcaagcctgaaagcggatgatactgccgtatacttctgtagccgcagtaggggcaaaaacgaagcgtggttcgcgtattggggacagggaactttggtcacagtctcatcc

SEQ ID NO: 60 - DNA, 741 bp, synthetic construct
  misc_feature 1..741: note = Description of sequence: 5319
  source 1..741: mol_type = other DNA
gacattcagatgacccaaactacttccagtctttcagcctcccttggtgatagggtgacgattagttgccgcgcttcacagtccctggtacacaggaatggcaatacttatcttaactggtaccaacaaaaacccgacggcaccgtcaaacttctcatctatacggtctctaggttgcattccggagtcccatcacggtttagtggttccggcagcggtacagactatagcctgactatctctaaccttgagcaagaagacatcgccacctacttttgctcacagagctcccacgtccctcctaccttcggcggtggtacaaaactggagatcaccgggggaggtggatctggcgggggtggatctggtgggggcggaagtcaggtgcagttgcagcaatccggttctgaacttaaaaagcctggggccagtgtaaaagtgtcatgtaaggcaagcgggtacacctttacaaattatggagtgaactggataaaacaggcccctgggcaaggattgcaatggatggggtggataaatcccaacaccggagagccgacattcgatgacgacttctggggcagatttgccttctccctcgatacgtcagtgtccactgcgtatcttcaaatttcctccttgaaagccgatgatactgccgtgtatttttgctcccggagccgggagaaaaacgaagcctggttcgcttattggggccagggtactttggttactgtaagttct

SEQ ID NO: 61 - DNA, 741 bp, synthetic construct
  misc_feature 1..741: note = Description of sequence: 5315
  source 1..741: mol_type = other DNA
gatatccaaatgacacagaccacctcatctcttagcgcctctctgggcgatcgagtcacgatttcctgtcgggcgtctcagtcattggtacacaggaatggtaatacctatttgaattggtaccaacaaaaacccgatggcacggtaaagctgctgatttatactgtctccaggctgcactccggagtcccgtctcggttcagcggtagtgggagcggcactgactactcacttacaatctccaaccttgaacaagaggatatagctacatatttctgtagtcagagcagccatgtcccacctaccttcggtggaggcacgaaattggaaattacaggtggtggtggctctggtggcggagggtctggtggaggcggatctcaggtccagcttcaacagagtggtagcgagctgaagaaacccggcgcttcagtaaaggtctcatgcaaggcatccgggtacacgtttacaaattatggcgttaattggatcaaacaagcgcccggtcaaggcctgcaatggatggggtggatcaacccaaatactggcgagccaactttcgacgatgatttttgggggcgattcgcttttagccttgacacatccgtatctacggcttatctccagatttcaagcctcaaggccgatgataccgcagtatatttttgctgtcgctgccgcgaaaagaatgaagcctggttcgcctattgggggcagggcacccttgtcactgtgtcatca

SEQ ID NO: 62 - DNA, 741 bp, synthetic construct
  misc_feature 1..741: note = Description of sequence: 571
  source 1..741: mol_type = other DNA
gacatccagttgacacaaagcccattgagtctgccggtcacactgggtcagccggcctccatttcatgcaggtcatcacaaagtgaggtacacaggaatggtaacacataccttcattggttccagcaaagacctggtcaatctccccgattgcttatttacacagtcagtaaccgctttagtggcgtcccagatcgcttctcaggctctggttccggcacggatttcacgctgaaaataagcagggttgaggccgaggatgtgggggtttatttttgcagccagtccagtcatgtcccaccaaccttcggggccggaactcggttggagattaaaggcggtggggggtctggcggagggggtagcggcggaggcggttcccaggttcagctccaacaaagcggaagtgaactgaagaaaccaggagcatccgtgaaagttagctgtaaagcgtcaggatatacgtttacgaattatggcgtgaattggatcaaacaagcccccgggcaagggctccaatggatgggatggataaaccctaacaccggggaaccaacttttgatgacgatttcaagggaaggtttgccttctctttggatacttctgttagtacagcttaccttcaaattagcagcttgaaggcggacgacacagcagtctacttctgtagtcggtctagagggaaaaatgaagcgtggtttgcatattgggggcagggcacattggtgacagttagttct

SEQ ID NO: 63 - DNA, 741 bp, synthetic construct
  misc_feature 1..741: note = Description of sequence: 573
  source 1..741: mol_type = other DNA
gatatacagcttacccaatcaccattgtccctcccagttaccttgggtcagccagcatctatatcttgcaggagcagccaatcattgtggcaccggaacggaaatacctaccttcattggtttcagcaaaggccgggacagtcaccgcgactgttgatatacaccgtctctaatcgattttccggtgtaccggatcgatttagcgggtctggatctggtacagattttacacttaagataagccgagtagaagcggaagatgtaggagtgtatttttgcagtcagagtagccacgttccgccaacgttcggagctggaacgaggttggagataaaagggggcggtggaagcggcggtggcggcagtggtggaggtggttctcaggtccaattgcagcagtctggctccgaactcaagaagccgggagccagcgtgaaagtaagctgcaaagcgagcggatatacgtttaccaattatggggtaaattggataaaacaagctcccggtcaagggttgcaatggatggggtggataaacccgaatacaggcgaaccgacatttgatgatgacttcaagggtcgattcgcctttagcttggacacatcagtgtctacggcctatcttcagatcagttccctcaaagcagatgataccgccgtgtatttttgttctcggtcacgcggtaaaaatgaggcatggttcgcttactgggggcaagggacgcttgtcacagtcagttca

SEQ ID NO: 64 - DNA, 741 bp, synthetic construct
  misc_feature 1..741: note = Description of sequence: 576
  source 1..741: mol_type = other DNA
gacatacaacttacccagtctccgctcagcctcccagtaaccctgggacaacctgcgagcatctcctgtaggtctagccagtccttggtccatcgcaacggaaacacctactgtcattggtttcagcaaaggcctggtcagtcccccaggctgcttatctatacggttagcaaccggtttagtggagtccccgatcgcttctccggtagcggaagtgggactgactttactctgaaaatttcacgagtggaggccgaagatgttggggtttatttttgcagtcagtctagtcatgttccaccaacctttggtgccggaacacggctcgaaatcaaaggaggtggcggcagcggaggtggcggctcagggggcggcgggtctcaagtgcaactgcagcaatctggttctgagctgaagaaacctggagcctcagttaaagtctcctgtaaggcttctgggtatacatttacaaactatggggtaaattggataaaacaggctccgggccaaggtcttcagtggatgggttggatcaatcccaacaccggtgaacctactttcgatgatgatttcaaaggacggtttgcattctctcttgacacttccgtgagcacagcttacctccaaatttcctcccttaaagctgatgatacagccgtgtatttttgttcccggagccggggtaagaacgaagcctggttcgcatattggggtcaaggcacacttgtaacagttagtagt

SEQ ID NO: 65 - DNA, 741 bp, synthetic construct
  misc_feature 1..741: note = Description of sequence: 578
  source 1..741: mol_type = other DNA
gacatccagttgacgcagtctccattgtccctgcccgtaaccttgggacaacctgcgtcaattagctgccgatcaagccaatcccttgttcaccggaacgggaacacgtatcttcactggttccagcaacgccctgggcagagtccgcggttgctgatctatgaggtcagcaaccgattttctggcgttccggaccgcttctccggcagtgggagtggcacggacttcaccctcaagatcagccgggtcgaagccgaggacgtgggcgtatatttctgctctcaaagctctcatgttccacctacgtttggcgcagggacgaggctcgagatcaaagggggtgggggctcaggcggtggcggaagcggcggaggcggcagtcaagtccagttgcagcaatcaggtagcgaactcaaaaagcccggcgcaagtgttaaagtatcctgtaaggctagcggctacacattcactaattatggggtaaactggatcaaacaagcgcccggtcaaggactccagtggatgggctggattaatcccaacacaggtgaaccgactttcgacgacgattttaagggaagatttgcattctctctcgatacctcagtctccaccgcctatctccagatcagtagccttaaagccgacgacaccgctgtgtatttctgttcccgatctcgaggaaagaacgaagcgtggttcgcctattgggggcaaggtacgttggtgaccgtatcctcc

SEQ ID NO: 66 - DNA, 741 bp, synthetic construct
  misc_feature 1..741: note = Description of sequence: 579
  source 1..741: mol_type = other DNA
gatatccaacttacccaatctcccctcagcttgccagtcacgcttggccaacccgccagcatttcctgtaggtcatcacagagtctcgtccataggaatggtaatacatacttgcattggtttcaacaaagacccgggcaaagtccacggctgctcatatacacagagagcaacagattttctggggttccggataggttttctggctctggtagtggaacagactttactcttaagatcagtagggtcgaagctgaggatgtaggggtttacttttgctcccagtccagccatgtcccaccaacctttggcgcaggcacgagattggagattaagggcggcgggggcagcggtggaggcgggagcggtggaggaggttctcaggtgcagcttcaacagtcaggatcagagctcaagaagcctggagccagcgtgaaagtatcttgtaaggcctccggatacacctttaccaattatggggttaattggataaaacaagcgcctgggcaagggttgcaatggatgggatggataaaccctaacaccggagagccgactttcgacgatgatttcaaaggtcggtttgcattctccttggacacgtccgtatcaacagcatatctccaaatttcatccctcaaggcagacgacacggcggtgtatttttgtagtaggagtcgaggcaagaacgaagcatggttcgcatattgggggcaaggcacgcttgttacggtgagctct

SEQ ID NO: 67 - DNA, 741 bp, synthetic construct
  misc_feature 1..741: note = Description of sequence: 5711
  source 1..741: mol_type = other DNA
gacatccagcttacccagagtccactgagccttcccgtaacgctcggtcaacccgcaagtatatcttgccgctctagtcaaagtctggtccatcggaacggtaatacatatttgcactggtttcaacagagaccagggcaaagtcccaggctcttgatctacactgtaagtaacagattcagcggtgttccggacaggttcagcggcagtggctccggtactgatttcaccctgaaaatcagtagagttgaagcggaggacgtaggcgtatacttctgcagctgtagcagtcacgtacctcctacgtttggagccggcacgcggctcgagattaaaggaggcggcggctccggtggtggtggcagcggtggaggggggagtcaagtgcaactccaacagtcagggagcgagctcaaaaaacctggggcaagcgtcaaagtatcctgcaaagcaagtgggtacacattcaccaactatggggtcaactggattaaacaagctccggggcagggtcttcagtggatgggatggataaacccgaatactggggagcccactttcgacgacgacttcaaagggcgatttgccttctccctcgacaccagcgttagtacggcgtatttgcaaatatcatccctcaaagcagatgataccgctgtgtatttctgttccagaagcaggggaaagaacgaggcttggtttgcatattggggtcaaggcacgctcgttacagtcagttcc

SEQ ID NO: 68 - DNA, 741 bp, synthetic construct
  misc_feature 1..741: note = Description of sequence: 5718
  source 1..741: mol_type = other DNA
gatatacagcttacccagtcccccctgagtttgcctgtcactttgggtcagccggcttcaatctcatgcaggtcttctcagtctctggttcaccggaatggtaacacttatcttcattggtttcagcagcggcctggtcagtcaccgagattgttgatttataccgtgtcaaacaggttttcaggtgtacctgacaggttttcaggaagtggctctggaactgactttacattgaaaatatcccgcgttgaggcggaagatgtgggggtgtacttttgtagtcagagttctcattggccccccacattcggcgcgggcacacgattggagataaaaggcgggggaggcagcggtgggggaggatcaggtggaggtgggagtcaggttcagctgcaacaatcagggagtgagttgaagaaaccaggcgcttccgtcaaggtcagctgtaaagcgtcaggatatacttttacaaactacggagtcaattggattaagcaggctcctggtcaggggttgcaatggatgggatggataaatccaaacacgggtgaacctacattcgacgacgacttcaagggacgattcgcattttcacttgacacgagtgtgagcacagcctatctccaaatctcctcactgaaggcggatgatactgctgtgtatttctgctcacgatccaggggtaaaaacgaagcatggtttgcgtactgggggcagggcacacttgtcacggtatcatcc

SEQ ID NO: 69 - DNA, 741 bp, synthetic construct
  misc_feature 1..741: note = Description of sequence: 5721
  source 1..741: mol_type = other DNA
gacatccaactgacacaatccccactgagtttgccagtcacgctgggacaacccgcatctatatcctgccggtcctcacaaagtttggtacatcgcaacggtaatacttacttgcattggtttcaacaacgaccggggcaatcacccagacttctcatatacacggtaagcaaccgatttagtggagtacctgacagattctctggctctggtagcgggaccgatttcactctcaaaataagcagagtagaagcggaggatgtgggcgtatatttttgtagtcaatctagtcatgttccaccttgctttggagccggtactcgactcgaaattaaaggtggagggggttctggaggaggcggaagcggaggaggtggttcccaagttcagctgcaacaaagtggaagtgaattgaagaagcctggtgcatctgtcaaagtgtcctgcaaagcctctggctacacattcacgaactatggcgttaattggatcaagcaggcgccgggtcaaggccttcaatggatggggtggataaatccgaatacgggggaacccacgtttgacgatgacttcaagggtaggtttgctttttctcttgacacctccgtttctactgcctatcttcaaataagctcactcaaagctgatgacacagccgtttatttctgcagtcgcagtcgagggaaaaacgaggcgtggtttgcgtattggggccagggcacccttgtaacggtgtccagt

SEQ ID NO: 70 - DNA, 741 bp, synthetic construct
  misc_feature 1..741: note = Description of sequence: 532
  source 1..741: mol_type = other DNA
gatatacagctgacgcaatctccgctgagtctccccgttactctgggccaacctgcatccattagttgtcgctccagtcagtcacttgtacaccgaaatggaaacacctacttgcactggttccagcaaagacccggccagtccccacgccttctcatatatactgtaagtaatcgcttttcaggtgtgcctgataggtttagcgggagcgggagcggcacggacttcacacttaaaatttctagagtagaggccgaggacgtaggtgtgtacttttgttcccagtctagccacgtcccacctacctttggggcggggaccagattggagattaagggcggcggagggagtggtggagggggctcaggaggaggcggcagccaagttcaactgcagcagagtggttccgagctcaagaagccgggcgcttctgttaaggtctcatgtaaagcttccggttatacatttacgaactatggtatgaattggatcaagcaagcacccggacagggattgcaatggatgggctggataaatcccaatacaggtgagcccacgttcgatgacgacttcaaaggtcggtttgccttttccctggacacatccgtcagtaccgcgtaccttcagatttctagcctcaaggccgacgataccgcggtctacttctgttcccgctctcgcggaaagaatgaggcctggtttgcatactggggtcagggaacactcgttaccgtttcctct

SEQ ID NO: 71 - DNA, 741 bp, synthetic construct
  misc_feature 1..741: note = Description of sequence: 536
  source 1..741: mol_type = other DNA
gacattcaactcacacagagcccgctgtctctcccagtcacgctgggtcaaccagcatcaatctcatgtagatcaagccaatcactggtgcataggaatggaaacacgtaccttcactggttccaacagaggcccgggcaaagccctcggctgcttatatatactgtatcaaatcggttcagcggagtaccggaccgatttagtggatccgggagcgggaccgacttcaccctcaaaatctcccgagtcgaagccgaagacgtaggtgtttacttctgctcacagtcaagtcacgtccccccgacgtttggcgcgggtacacgactcgagattaagggtggaggcggaagcggcggtggagggagcggcggtggcggcagtcaagttcaattgcaacagagtggatccgagctgaaaaagcctggggcttcagtaaaagttagttgtaaggcaagtgggtatacttttactaattactgggtgaattggataaaacaagccccgggccaaggccttcagtggatgggttggattaatcccaatacgggcgagcccactttcgacgacgattttaagggcagattcgcattctctctggacacgtcagtaagcaccgcgtatttgcaaattagctcccttaaagcggacgacacggcggtatatttttgctctagaagtcgcggcaaaaacgaagcctggtttgcttactggggccagggcaccttggtcacggtctcctct

SEQ ID NO: 72 - DNA, 741 bp, synthetic construct
  misc_feature 1..741: note = Description of sequence: 537
  source 1..741: mol_type = other DNA
gacatacagcttacgcaatcacctttgtcccttccagtgacgcttggccaacctgcctccataagctgcagatcctcccagtcactggtacatagaaatggcaatacctatttgcactggttccagcagcggcccggccaatcccctcgacttctcatatacaccgtgtcaaaccgcttttccggcgtgccggaccgattcagtggctctggctctggcacggactttactctcaaaatctctagagtcgaggcggaggacgttggcgtttatttttgttcacagtccagccatgtccctccgactttcggggcgggaacaagactggagattaagggaggaggtggctcaggtggtggtggatcagggggaggtggatcacaagttcaactccaacagtctggaagtgagttgaagaaacccggagcttcagtcaaggtctcctgtaaagcatccggttacacgttcaccaattatggagttaattggatcaagcaggcgccgggccagggccttcagtggatgggctggatcaacccaaatacaggtgaacccacgttcgatgatgattttaagggtcgatttgcattttccttggatacatctgtgtctacggcatatttgcaaataagcagtctcaaggctgacgatactgccgtgtacttctgctccaggtcaagaggaaagaacgaggcgtggttcgcctactgggggcaaggaaccttggtaacggttagtagt

SEQ ID NO: 73 - DNA, 741 bp, synthetic construct
  misc_feature 1..741: note = Description of sequence: 5313
  source 1..741: mol_type = other DNA
gatatacagttgacccaaagtcccctgtctttgcccgtcactctcggccagcccgcatccatttcttgtcggtcctctcaaagtctggtccacaggaatggtaatacctacttgcactggttccagcaacggccgggccaaagtccgcggctgctgatatacacagtgagcaataggttttcaggcgtaccggacaggttctctggttccggctctggtactgatttcacgctgaaaatctcacgagtggaagctgaggatgttggggtctatttctgcagtcagtcctcccatgtcccaccgacatttggagctgggacaagactcgagatcaaaggcggcggggggagtggagggggtgggagtggtggaggaggtagccaagtccagcttcagcaaagtggatctgaattgaagaaaccgggagcctctgtcaaggtgagctgcaaagctagcggctatacctttacaaattacggagtcaactggataaagcaagcccctggtcaggggctccaatggatgggctggatcaaccctaataccggtgaacccacgtgggacgatgatttcaagggaagattcgcattttccctcgacacgtcagtcagtacggcctacctccaaataagctccctcaaagctgacgatacggcggtctatttttgttccagatcccgagggaagaacgaggcatggtttgcttactggggtcaaggcactttggttaccgtcagtagc

SEQ ID NO: 74 - protein, 5 aa, synthetic construct
  REGION 1..5: note = Description of sequence: Antibody, sequence: 42105-01, Variable Region: VH, CDR1
NYGVS

SEQ ID NO: 75 - protein, 17 aa, synthetic construct
  REGION 1..17: note = Description of sequence: Antibody, sequence: 42105-01, Variable Region: VH, CDR2
VINPNTGEPYYNSALKS

SEQ ID NO: 76 - protein, 11 aa, synthetic construct
  REGION 1..11: note = Description of sequence: Antibody, sequence: 42105-01, Variable Region: VH, CDR3
SRGKNEAWFAY

SEQ ID NO: 77 - protein, 16 aa, synthetic construct
  REGION 1..16: note = Description of sequence: Antibody, sequence: 42105-01, Variable Region: VL, CDR1
RASQSLVHRNGNTYLN

SEQ ID NO: 78 - protein, 7 aa, synthetic construct
  REGION 1..7: note = Description of sequence: Antibody, sequence: 42105-01, Variable Region: VL, CDR2
TVSRLHS

SEQ ID NO: 79 - protein, 9 aa, synthetic construct
  REGION 1..9: note = Description of sequence: Antibody, sequence: 42105-01, Variable Region: VL, CDR3
SQSSHVPPT

SEQ ID NO: 80 - protein, 5 aa, synthetic construct
  REGION 1..5: note = Description of sequence: Antibody, sequence: 5311-01, Variable Region: VH, CDR1
NYGVN

SEQ ID NO: 81 - protein, 17 aa, synthetic construct
  REGION 1..17: note = Description of sequence: Antibody, sequence: 5311-01, Variable Region: VH, CDR2
WINPNTGEPTYDDDFKG

SEQ ID NO: 82 - protein, 11 aa, synthetic construct
  REGION 1..11: note = Description of sequence: Antibody, sequence: 5311-01, Variable Region: VH, CDR3
SRGKNEAWFAY

SEQ ID NO: 83 - protein, 16 aa, synthetic construct
  REGION 1..16: note = Description of sequence: Antibody, sequence: 5311-01, Variable Region: VL, CDR1
RASQSLVHRNGNTYLN

SEQ ID NO: 84 - protein, 7 aa, synthetic construct
  REGION 1..7: note = Description of sequence: Antibody, sequence: 5311-01, Variable Region: VL, CDR2
TVSRLHS

SEQ ID NO: 85 - protein, 9 aa, synthetic construct
  REGION 1..9: note = Description of sequence: Antibody, sequence: 5311-01, Variable Region: VL, CDR3
SQSSHVPPT

SEQ ID NO: 86 - protein, 5 aa, synthetic construct
  REGION 1..5: note = Description of sequence: Antibody, sequence: 543, Variable Region: VH, CDR1
SYGMH

SEQ ID NO: 87 - protein, 20 aa, synthetic construct
  REGION 1..20: note = Description of sequence: Antibody, sequence: 543, Variable Region: VH, CDR2
WVAVIWYDGSNKYYADSVKG

SEQ ID NO: 88 - protein, 14 aa, synthetic construct
  REGION 1..14: note = Description of sequence: Antibody, sequence: 543, Variable Region: VH, CDR3
GGTLVRGAMYGTDV

SEQ ID NO: 89 - protein, 15 aa, synthetic construct
  REGION 1..15: note = Description of sequence: Antibody, sequence: 543, Variable Region: VL, CDR1
TITCRASQGISSWLA

SEQ ID NO: 90 - protein, 7 aa, synthetic construct
  REGION 1..7: note = Description of sequence: Antibody, sequence: 543, Variable Region: VL, CDR2
AASSLQS

SEQ ID NO: 91 - protein, 9 aa, synthetic construct
  REGION 1..9: note = Description of sequence: Antibody, sequence: 543, Variable Region: VL, CDR3
QQYNSYPLT

SEQ ID NO: 92 - DNA, 672 bp, synthetic construct
  misc_feature 1..672: note = Description of sequence: CAR backbone
  source 1..672: mol_type = other DNA
acaacaactccggctccacggcctcctaccccggcccccacaatcgcttcacaaccactgtcactcaggcctgaagcatgtcggcccgccgctggcggtgctgtgcacacacgaggcttggacttcgcctgcgacatttacatatgggcacctttggccggcacctgcggcgtcctccttctgagcctcgtcataacgctgtattgcaagcgaggtcggaagaagttgctctatatattcaagcaaccgttcatgcgaccggtacagacgacccaggaagaggatggctgttcatgtcggttcccagaggaagaagagggtggctgcgagcttagggtcaaattttccagaagcgctgatgcgcctgcttaccagcaaggacagaaccaactttataacgaactgaatctcggacgccgggaagaatatgatgtgttggataaacggaggggccgcgacccggagatgggaggcaagccaagacgcaaaaacccacaggagggtctttacaatgaactccagaaagacaaaatggccgaggcttatagtgagataggcatgaagggcgagagaagacgaggtaagggacacgacggactgtatcaagggctgtccacagctaccaaggacacctacgacgccctccacatgcaggctctcccgccccgatag